SEQUENCE LISTING (456 sequences)

SEQ ID NO: 1 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tcccagaaccagggaggcatt

SEQ ID NO: 2 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cttttgagaccctgctgtctt

SEQ ID NO: 3 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ctgtcccagaaccagggagtt

SEQ ID NO: 4 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgtcccagaaccagggaggtt

SEQ ID NO: 5 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
aagccttttgagaccctgctt

SEQ ID NO: 6 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ttgagaccctgctgtcccatt

SEQ ID NO: 7 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ttttgagaccctgctgtcctt

SEQ ID NO: 8 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gagaccctgctgtcccagatt

SEQ ID NO: 9 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gctggaaggaggaggtggttt

SEQ ID NO: 10 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ctggaaggaggaggtggtgtt

SEQ ID NO: 11 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tcagggccagagctggaagtt

SEQ ID NO: 12 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgagaccctgctgtcccagtt

SEQ ID NO: 13 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
agggccagagctggaaggatt

SEQ ID NO: 14 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
agctggaaggaggaggtggtt

SEQ ID NO: 15 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
agaccctgctgtcccagaatt

SEQ ID NO: 16 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gagctggaaggaggaggtgtt

SEQ ID NO: 17 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgctgtcccagaaccagggtt

SEQ ID NO: 18 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cccagaaccagggaggcaatt

SEQ ID NO: 19 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ccagaaccagggaggcaagtt

SEQ ID NO: 20 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tttgagaccctgctgtccctt

SEQ ID NO: 21 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gaccctgctgtcccagaactt

SEQ ID NO: 22 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gatcagggccagagctggatt

SEQ ID NO: 23 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
agccttttgagaccctgcttt

SEQ ID NO: 24 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gccttttgagaccctgctgtt

SEQ ID NO: 25 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ccttttgagaccctgctgttt

SEQ ID NO: 26 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cgccttttgagaccctgcatt

SEQ ID NO: 27 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cctacaccgtggtctattttt

SEQ ID NO: 28 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgtgggagaccagatctcctt

SEQ ID NO: 29 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gcgggaggcagagtttgcctt

SEQ ID NO: 30 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cctttctccaggaccaatatt

SEQ ID NO: 31 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
accctgctgtcccagaacctt

SEQ ID NO: 32 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ggtctatttcccagttcgatt

SEQ ID NO: 33 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ccctggtggacatggtgaatt

SEQ ID NO: 34 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
acatctccctcatctacactt

SEQ ID NO: 35 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gcaaggatgactatgtgaatt

SEQ ID NO: 36 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ccttcgctgactacaaccttt

SEQ ID NO: 37 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ctggcagatcagggccagatt

SEQ ID NO: 38 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gacggagacctcaccctgttt

SEQ ID NO: 39 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cgggcaaggatgactatgttt

SEQ ID NO: 40 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cttttgagaccctgctgtatt

SEQ ID NO: 41 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gagctggaaggaggaggtatt

SEQ ID NO: 42 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
accctgctgtcccagaacatt

SEQ ID NO: 43 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgctgtcccagaaccaggatt

SEQ ID NO: 44 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
agccttttgagaccctgcatt

SEQ ID NO: 45 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ccttttgagaccctgctgatt

SEQ ID NO: 46 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgaagccttttgagacccttt

SEQ ID NO: 47 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
actgaagccttttgagacctt

SEQ ID NO: 48 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
aggatgactatgtgaaggctt

SEQ ID NO: 49 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ggatgactatgtgaaggcatt

SEQ ID NO: 50 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gatgactatgtgaaggcactt

SEQ ID NO: 51 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ctccctcatctacaccaactt

SEQ ID NO: 52 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gaagccttttgagaccctgtt

SEQ ID NO: 53 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tctccctcatctacaccaatt

SEQ ID NO: 54 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cctcatctacaccaactattt

SEQ ID NO: 55 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ccctcatctacaccaactatt

SEQ ID NO: 56 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
caactgaagccttttgagatt

SEQ ID NO: 57 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
aactgaagccttttgagactt

SEQ ID NO: 58 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ctgaagccttttgagaccctt

SEQ ID NO: 59 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tccctcatctacaccaacttt

SEQ ID NO: 60 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gctccctcatctacaccaatt

SEQ ID NO: 61 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gaagccttttgagaccctatt

SEQ ID NO: 62 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
actgaagccttttgagacatt

SEQ ID NO: 63 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ctccctcatctacaccaaatt

SEQ ID NO: 64 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cctcatctacaccaactaatt

SEQ ID NO: 65 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
accaataaaatttctaagatt

SEQ ID NO: 66 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgcctccctggttctgggaca

SEQ ID NO: 67 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gacagcagggtctcaaaaggc

SEQ ID NO: 68 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ctccctggttctgggacagca

SEQ ID NO: 69 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cctccctggttctgggacagc

SEQ ID NO: 70 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gcagggtctcaaaaggcttca

SEQ ID NO: 71 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgggacagcagggtctcaaaa

SEQ ID NO: 72 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ggacagcagggtctcaaaagg

SEQ ID NO: 73 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tctgggacagcagggtctcaa

SEQ ID NO: 74 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
accacctcctccttccagctc

SEQ ID NO: 75 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
caccacctcctccttccagct

SEQ ID NO: 76 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cttccagctctggccctgatc

SEQ ID NO: 77 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ctgggacagcagggtctcaaa

SEQ ID NO: 78 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tccttccagctctggccctga

SEQ ID NO: 79 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ccacctcctccttccagctct

SEQ ID NO: 80 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ttctgggacagcagggtctca

SEQ ID NO: 81 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cacctcctccttccagctctg

SEQ ID NO: 82 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ccctggttctgggacagcagg

SEQ ID NO: 83 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ttgcctccctggttctgggac

SEQ ID NO: 84 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cttgcctccctggttctggga

SEQ ID NO: 85 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gggacagcagggtctcaaaag

SEQ ID NO: 86 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gttctgggacagcagggtctc

SEQ ID NO: 87 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tccagctctggccctgatctg

SEQ ID NO: 88 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
agcagggtctcaaaaggcttc

SEQ ID NO: 89 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cagcagggtctcaaaaggctt

SEQ ID NO: 90 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
acagcagggtctcaaaaggct

SEQ ID NO: 91 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgcagggtctcaaaaggcgtc

SEQ ID NO: 92 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
aaatagaccacggtgtagggc

SEQ ID NO: 93 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ggagatctggtctcccacaat

SEQ ID NO: 94 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ggcaaactctgcctcccgctc

SEQ ID NO: 95 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tattggtcctggagaaaggaa

SEQ ID NO: 96 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ggttctgggacagcagggtct

SEQ ID NO: 97 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tcgaactgggaaatagaccac

SEQ ID NO: 98 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ttcaccatgtccaccagggct

SEQ ID NO: 99 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gtgtagatgagggagatgtat

SEQ ID NO: 100 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ttcacatagtcatccttgccc

SEQ ID NO: 101 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
aggttgtagtcagcgaaggag

SEQ ID NO: 102 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tctggccctgatctgccagca

SEQ ID NO: 103 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
acagggtgaggtctccgtcct

SEQ ID NO: 104 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
acatagtcatccttgcccgcc

SEQ ID NO: 105 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tacagcagggtctcaaaaggc

SEQ ID NO: 106 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tacctcctccttccagctctg

SEQ ID NO: 107 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgttctgggacagcagggtct

SEQ ID NO: 108 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tcctggttctgggacagcagg

SEQ ID NO: 109 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgcagggtctcaaaaggcttc

SEQ ID NO: 110 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tcagcagggtctcaaaaggct

SEQ ID NO: 111 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
agggtctcaaaaggcttcagt

SEQ ID NO: 112 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ggtctcaaaaggcttcagttg

SEQ ID NO: 113 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gccttcacatagtcatccttg

SEQ ID NO: 114 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgccttcacatagtcatcctt

SEQ ID NO: 115 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gtgccttcacatagtcatcct

SEQ ID NO: 116 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gttggtgtagatgagggagat

SEQ ID NO: 117 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
cagggtctcaaaaggcttcag

SEQ ID NO: 118 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ttggtgtagatgagggagatg

SEQ ID NO: 119 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
atagttggtgtagatgaggga

SEQ ID NO: 120 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagttggtgtagatgagggag

SEQ ID NO: 121 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tctcaaaaggcttcagttgcc

SEQ ID NO: 122 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gtctcaaaaggcttcagttgc

SEQ ID NO: 123 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gggtctcaaaaggcttcagtt

SEQ ID NO: 124 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
agttggtgtagatgagggaga

SEQ ID NO: 125 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ttggtgtagatgagggagctg

SEQ ID NO: 126 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagggtctcaaaaggcttcag

SEQ ID NO: 127 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tgtctcaaaaggcttcagttg

SEQ ID NO: 128 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tttggtgtagatgagggagat

SEQ ID NO: 129 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ttagttggtgtagatgaggga

SEQ ID NO: 130 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tcttagaaattttattggtcc

SEQ ID NO: 131 - RNA, 21 nt, synthetic construct
  misc_feature 1..21: note = Description of Artificial Sequence: Synthetic oligonucleotide
  misc_difference 20..21: note = one or more of the "n" positions may be absent, and if absent, the terminal residue may be connected to an inverted nucleotide via a 3' to 3' bond; note = any ribo-nucleotide or 2'-deoxy-nucleotide, 2'-OMe-U, or a modified or chemically modified nucleotide
  source 1..21: mol_type = other RNA
gaagccttttgagaccctann

SEQ ID NO: 132 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 133 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 134 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 135 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 136 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 137 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 138 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 139 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 140 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 141 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 142 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gaagccttttgagaccctatt

SEQ ID NO: 143 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 144 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 145 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 146 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctatt

SEQ ID NO: 147 - RNA, 21 nt, synthetic construct
  misc_feature 1..21: note = Description of Artificial Sequence: Synthetic oligonucleotide
  modified_base 19: mod_base = OTHER; note = 2'-deoxy-2'-fluoro-nucleotide
  modified_base 20..21: mod_base = OTHER; note = 2'-OMe-nucleotide
  source 1..21: mol_type = other RNA
gaagccttttgagaccctatt

SEQ ID NO: 148 - RNA, 21 nt, synthetic construct
  misc_feature 1..21: note = Description of Artificial Sequence: Synthetic oligonucleotide
  modified_base 14: mod_base = OTHER; note = 2'-deoxy-2'-fluoro-nucleotide
  modified_base 19: mod_base = OTHER; note = 2'-deoxy-2'-fluoro-nucleotide
  modified_base 20..21: mod_base = OTHER; note = 2'-OMe-nucleotide
  source 1..21: mol_type = other RNA
gaagccttttgagaccctatt

SEQ ID NO: 149 - RNA, 21 nt, synthetic construct
  misc_feature 1..21: note = Description of Artificial Sequence: Synthetic oligonucleotide
  modified_base 19..21: mod_base = OTHER; note = 2'-OMe-nucleotide
  source 1..21: mol_type = other RNA
gaagccttttgagaccctatt

SEQ ID NO: 150 - RNA, 21 nt, synthetic construct
  misc_feature 1..21: note = Description of Artificial Sequence: Synthetic oligonucleotide
  modified_base 19..21: mod_base = OTHER; note = 2'-OMe-nucleotide
  source 1..21: mol_type = other RNA
gaagccttttgagaccctatt

SEQ ID NO: 151 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 20..21: mod_base = OTHER; note = 2'-OMe-nucleotide
  source 1..21: mol_type = other RNA
gaagccttttgagaccctatt

SEQ ID NO: 152 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 8: mod_base = OTHER; note = 2'-OMe-nucleotide
  modified_base 10: mod_base = OTHER; note = 2'-OMe-nucleotide
  modified_base 18: mod_base = OTHER; note = 2'-OMe-nucleotide
  modified_base 20..21: mod_base = OTHER; note = 2'-OMe-nucleotide
  source 1..21: mol_type = other RNA
gaagccttttgagaccctatt

SEQ ID NO: 153 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 20..21: mod_base = OTHER; note = 2'-OMe-nucleotide
  source 1..21: mol_type = other RNA
gaagccttttgagaccctatt

SEQ ID NO: 154 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 8: mod_base = OTHER; note = 2'-OMe-nucleotide
  modified_base 10: mod_base = OTHER; note = 2'-OMe-nucleotide
  modified_base 18: mod_base = OTHER; note = 2'-OMe-nucleotide
  modified_base 20..21: mod_base = OTHER; note = 2'-OMe-nucleotide
  source 1..21: mol_type = other RNA
gaagccttttgagaccctatt

SEQ ID NO: 155 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 8: mod_base = OTHER; note = 2'-OMe-nucleotide
  modified_base 10: mod_base = OTHER; note = 2'-OMe-nucleotide
  modified_base 18..21: mod_base = OTHER; note = 2'-OMe-nucleotide
  source 1..21: mol_type = other RNA
gaagccttttgagaccctatt

SEQ ID NO: 156 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 8: mod_base = OTHER; note = 2'-OMe-nucleotide
  modified_base 10: mod_base = OTHER; note = 2'-OMe-nucleotide
  modified_base 18..21: mod_base = OTHER; note = 2'-OMe-nucleotide
  source 1..21: mol_type = other RNA
gaagccttttgagaccctatt

SEQ ID NO: 157 - RNA, 21 nt, synthetic construct
  misc_feature 1..21: note = Description of Artificial Sequence: Synthetic oligonucleotide
  misc_difference 20..21: note = any ribo-nucleotide or 2'-deoxy-nucleotide, 2'-OMe-U, or a modified or chemically modified nucleotide; note = one or more of the "n" positions may be absent, and if absent, the terminal residue may be connected to an inverted nucleotide via a 3' to 3' bond
  source 1..21: mol_type = other RNA
tagggtctcaaaaggcttcnn

SEQ ID NO: 158 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
tagggtctcaaaaggcttctt

SEQ ID NO: 159 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 1: mod_base = OTHER; note = 2'-deoxy-nucleotide (2'-deoxyuridine)
  modified_base 6: mod_base = OTHER; note = 2'-deoxy-nucleotide (2'-deoxyuridine)
  modified_base 8: mod_base = OTHER; note = 2'-deoxy-nucleotide (2'-deoxyuridine)
  source 1..21: mol_type = other DNA
  misc_feature 1..8: note = DNA
  misc_feature 9..21: note = RNA
tagggtctcaaaaggcttctt

SEQ ID NO: 160 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  misc_feature 1: note = RNA
  modified_base 6: mod_base = OTHER; note = 2'-deoxy-nucleotide (2'-deoxyuridine)
  modified_base 8: mod_base = OTHER; note = 2'-deoxy-nucleotide (2'-deoxyuridine)
  source 1..21: mol_type = other DNA
  misc_feature 1: note = RNA
  misc_feature 2..8: note = DNA
  misc_feature 9..21: note = RNA
tagggtctcaaaaggcttctt

SEQ ID NO: 161 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 6: mod_base = OTHER; note = 2'-deoxy-nucleotide (2'-deoxyuridine)
  modified_base 8: mod_base = OTHER; note = 2'-deoxy-nucleotide (2'-deoxyuridine)
  source 1..21: mol_type = other DNA
  misc_feature 1..2: note = RNA
  misc_feature 3..8: note = DNA
  misc_feature 9..21: note = RNA
tagggtctcaaaaggcttctt

SEQ ID NO: 162 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 6: mod_base = OTHER; note = 2'-deoxy-nucleotide (2'-deoxyuridine)
  modified_base 8: mod_base = OTHER; note = 2'-deoxy-nucleotide (2'-deoxyuridine)
  source 1..21: mol_type = other DNA
  misc_feature 1..3: note = RNA
  misc_feature 4..8: note = DNA
  misc_feature 9..21: note = RNA
tagggtctcaaaaggcttctt

SEQ ID NO: 163 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 6: mod_base = OTHER; note = 2'-deoxy-nucleotide (2'-deoxyuridine)
  modified_base 8: mod_base = OTHER; note = 2'-deoxy-nucleotide (2'-deoxyuridine)
  source 1..21: mol_type = other DNA
  misc_feature 1..4: note = RNA
  misc_feature 5..8: note = DNA
  misc_feature 9..21: note = RNA
tagggtctcaaaaggcttctt

SEQ ID NO: 164 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  misc_feature 2: note = RNA
  misc_feature 4: note = RNA
  modified_base 1: mod_base = OTHER; note = 2'-deoxy-nucleotide (2'-deoxyuridine)
  misc_feature 6: note = RNA
  source 1..21: mol_type = other DNA
  misc_feature 1: note = DNA
  misc_feature 2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
tagggtctcaaaaggcttctt

SEQ ID NO: 165 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  misc_feature 4: note = RNA
  misc_feature 6: note = RNA
  source 1..21: mol_type = other DNA
  misc_feature 1..2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
tagggtctcaaaaggcttctt

SEQ ID NO: 166 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  misc_feature 1: note = RNA
  misc_feature 3: note = RNA
  misc_feature 5: note = RNA
  modified_base 6: mod_base = OTHER; note = 2'-deoxy-nucleotide (2'-deoxyuridine)
  misc_feature 7: note = RNA
  modified_base 8: mod_base = OTHER; note = 2'-deoxy-nucleotide (2'-deoxyuridine)
  source 1..21: mol_type = other DNA
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
tagggtctcaaaaggcttctt

SEQ ID NO: 167 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  misc_feature 5: note = RNA
  modified_base 6: mod_base = OTHER; note = 2'-deoxy-nucleotide (2'-deoxyuridine)
  misc_feature 7: note = RNA
  modified_base 8: mod_base = OTHER; note = 2'-deoxy-nucleotide (2'-deoxyuridine)
  source 1..21: mol_type = other DNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
tagggtctcaaaaggcttctt

SEQ ID NO: 168 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 14: mod_base = OTHER; note = 2'-OMe-nucleotide
  modified_base 16: mod_base = OTHER; note = 2'-OMe-nucleotide
  modified_base 18: mod_base = OTHER; note = 2'-OMe-nucleotide
  modified_base 20..21: mod_base = OTHER; note = 2'-OMe-nucleotide
  misc_feature 1: note = RNA
  modified_base 6: mod_base = OTHER; note = 2'-deoxy-nucleotide (2'-deoxyuridine)
  modified_base 8: mod_base = OTHER; note = 2'-deoxy-nucleotide (2'-deoxyuridine)
  source 1..21: mol_type = other DNA
  misc_feature 1: note = RNA
  misc_feature 2..8: note = DNA
  misc_feature 9..21: note = RNA
tagggtctcaaaaggcttctt

SEQ ID NO: 169 - RNA, 21 nt, synthetic construct
  misc_feature 1..21: note = Description of Artificial Sequence: Synthetic oligonucleotide
  modified_base 1: mod_base = OTHER; note = 2'-OMe-nucleotide
  modified_base 20..21: mod_base = OTHER; note = 2'-OMe-nucleotide
  source 1..21: mol_type = other RNA
tagggtctcaaaaggcttctt

SEQ ID NO: 170 - RNA, 21 nt, synthetic construct
  misc_feature 1..21: note = Description of Artificial Sequence: Synthetic oligonucleotide
  modified_base 1: mod_base = OTHER; note = 2'-deoxy-2'-fluoro-nucleotide
  modified_base 20..21: mod_base = OTHER; note = 2'-OMe-nucleotide
  source 1..21: mol_type = other RNA
tagggtctcaaaaggcttctt

SEQ ID NO: 171 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 1: mod_base = OTHER; note = 2'-deoxy-nucleotide (2'-deoxyuridine)
  source 1..21: mol_type = other DNA
tagggtctcaaaaggcttctt

SEQ ID NO: 172 - RNA, 21 nt, synthetic construct
  misc_feature 1..21: note = Description of Artificial Sequence: Synthetic oligonucleotide
  modified_base 1..2: mod_base = OTHER; note = Phosphorothioate linkage
  modified_base 20..21: mod_base = OTHER; note = 2'-OMe-nucleotide
  source 1..21: mol_type = other RNA
tagggtctcaaaaggcttctt

SEQ ID NO: 173 - RNA, 21 nt, synthetic construct
  misc_feature 1..21: note = Description of Artificial Sequence: Synthetic oligonucleotide
  modified_base 1: mod_base = OTHER; note = 2'-deoxy-2'-fluoro-nucleotide
  modified_base 9: mod_base = OTHER; note = 2'-deoxy-2'-fluoro-nucleotide
  modified_base 17: mod_base = OTHER; note = 2'-deoxy-2'-fluoro-nucleotide
  modified_base 20..21: mod_base = OTHER; note = 2'-OMe-nucleotide
  source 1..21: mol_type = other RNA
tagggtctcaaaaggcttctt

SEQ ID NO: 174 - RNA, 21 nt, synthetic construct
  misc_feature 1..21: note = Description of Artificial Sequence: Synthetic oligonucleotide
  modified_base 1: mod_base = OTHER; note = 2'-deoxy-2'-fluoro-nucleotide
  modified_base 9: mod_base = OTHER; note = 2'-deoxy-2'-fluoro-nucleotide
  modified_base 11..12: mod_base = OTHER; note = 2'-deoxy-2'-fluoro-nucleotide
  modified_base 17: mod_base = OTHER; note = 2'-deoxy-2'-fluoro-nucleotide
  modified_base 20..21: mod_base = OTHER; note = 2'-OMe-nucleotide
  source 1..21: mol_type = other RNA
tagggtctcaaaaggcttctt

SEQ ID NO: 175 - RNA, 21 nt, synthetic construct
  misc_feature 1..21: note = Description of Artificial Sequence: Synthetic oligonucleotide
  modified_base 1: mod_base = OTHER; note = 2'-OMe-nucleotide
  modified_base 9: mod_base = OTHER; note = 2'-OMe-nucleotide
  modified_base 17: mod_base = OTHER; note = 2'-OMe-nucleotide
  modified_base 20..21: mod_base = OTHER; note = 2'-OMe-nucleotide
  source 1..21: mol_type = other RNA
tagggtctcaaaaggcttctt

SEQ ID NO: 176 - RNA, 21 nt, synthetic construct
  misc_feature 1..21: note = Description of Artificial Sequence: Synthetic oligonucleotide
  modified_base 1: mod_base = OTHER; note = 2'-deoxy-2'-fluoro-nucleotide
  modified_base 9: mod_base = OTHER; note = 2'-OMe-nucleotide
  modified_base 17: mod_base = OTHER; note = 2'-OMe-nucleotide
  modified_base 20..21: mod_base = OTHER; note = 2'-OMe-nucleotide
  source 1..21: mol_type = other RNA
tagggtctcaaaaggcttctt

SEQ ID NO: 177 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
tagggtctcaaaaggcttctt

SEQ ID NO: 178 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 6: mod_base = OTHER; note = 2'-deoxy-nucleotide [2'-deoxyuridine]
  modified_base 8: mod_base = OTHER; note = 2'-deoxy-nucleotide [2'-deoxyuridine]
  misc_feature 5: note = RNA
  misc_feature 7: note = RNA
  source 1..21: mol_type = other DNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
tagggtctcaaaaggcttctt

SEQ ID NO: 179 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 1: mod_base = OTHER; note = 2'-OMe-nucleotide
  modified_base 9: mod_base = OTHER; note = 2'-OMe-nucleotide
  modified_base 11..12: mod_base = OTHER; note = 2'-OMe-nucleotide
  modified_base 18: mod_base = OTHER; note = 2'-OMe-nucleotide
  modified_base 20..21: mod_base = OTHER; note = 2'-OMe-nucleotide
  modified_base 6: mod_base = OTHER; note = 2'-deoxy-nucleotide [2'-deoxyuridine]
  modified_base 8: mod_base = OTHER; note = 2'-deoxy-nucleotide [2'-deoxyuridine]
  misc_feature 5: note = RNA
  source 1..21: mol_type = other DNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
tagggtctcaaaaggcttctt

SEQ ID NO: 180 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 1: mod_base = OTHER; note = 2'-OMe-nucleotide
  modified_base 9: mod_base = OTHER; note = 2'-OMe-nucleotide
  modified_base 11..12: mod_base = OTHER; note = 2'-OMe-nucleotide
  modified_base 18: mod_base = OTHER; note = 2'-OMe-nucleotide
  modified_base 20..21: mod_base = OTHER; note = 2'-OMe-nucleotide
  modified_base 6: mod_base = OTHER; note = 2'-deoxy-nucleotide [2'-deoxyuridine]
  modified_base 8: mod_base = OTHER; note = 2'-deoxy-nucleotide [2'-deoxyuridine]
  misc_feature 5: note = RNA
  misc_feature 7: note = RNA
  source 1..21: mol_type = other DNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
tagggtctcaaaaggcttctt

SEQ ID NO: 181 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 1: mod_base = OTHER; note = 2'-OMe-nucleotide
  modified_base 9: mod_base = OTHER; note = 2'-OMe-nucleotide
  modified_base 11..12: mod_base = OTHER; note = 2'-OMe-nucleotide
  modified_base 17..18: mod_base = OTHER; note = 2'-OMe-nucleotide
  modified_base 20..21: mod_base = OTHER; note = 2'-OMe-nucleotide
  modified_base 6: mod_base = OTHER; note = 2'-deoxy-nucleotide [2'-deoxyuridine]
  modified_base 8: mod_base = OTHER; note = 2'-deoxy-nucleotide [2'-deoxyuridine]
  misc_feature 5: note = RNA
  misc_feature 7: note = RNA
  source 1..21: mol_type = other DNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
tagggtctcaaaaggcttctt

SEQ ID NO: 182 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 1: mod_base = OTHER; note = 2'-deoxy-2'-fluoro-nucleotide
  modified_base 9: mod_base = OTHER; note = 2'-OMe-nucleotide
  modified_base 11..12: mod_base = OTHER; note = 2'-OMe-nucleotide
  modified_base 17..18: mod_base = OTHER; note = 2'-OMe-nucleotide
  modified_base 20..21: mod_base = OTHER; note = 2'-OMe-nucleotide
  modified_base 6: mod_base = OTHER; note = 2'-deoxy-nucleotide [2'-deoxyuridine]
  modified_base 8: mod_base = OTHER; note = 2'-deoxy-nucleotide [2'-deoxyuridine]
  misc_feature 5: note = RNA
  misc_feature 7: note = RNA
  source 1..21: mol_type = other DNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
tagggtctcaaaaggcttctt

SEQ ID NO: 183 - RNA, 21 nt, synthetic construct
  misc_feature 1..21: note = Description of Artificial Sequence: Synthetic oligonucleotide
  misc_difference 20..21: note = any ribo-nucleotide or 2'-deoxy-nucleotide, 2'-OMe-U, or a modified or chemically modified nucleotide; note = one or more of the "n" positions may be absent, and if absent, the terminal residue may be connected to an inverted nucleotide via a 3' to 3' bond
  source 1..21: mol_type = other RNA
ccttttgagaccctgctgtnn

SEQ ID NO: 184 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 185 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 186 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 187 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 188 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 189 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 190 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 191 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 192 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 193 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 194 - RNA, 21 nt, synthetic construct
  misc_feature 1..21: note = Description of Artificial Sequence: Synthetic oligonucleotide
  modified_base 1: mod_base = OTHER; note = 2'-OMe-nucleotide
  modified_base 3: mod_base = OTHER; note = 2'-OMe-nucleotide
  modified_base 5: mod_base = OTHER; note = 2'-OMe-nucleotide
  modified_base 7: mod_base = OTHER; note = 2'-OMe-nucleotide
  modified_base 9: mod_base = OTHER; note = 2'-OMe-nucleotide
  modified_base 20..21: mod_base = OTHER; note = 2'-OMe-nucleotide
  source 1..21: mol_type = other RNA
cctcatctacaccaactattt

SEQ ID NO: 195 - RNA, 21 nt, synthetic construct
  misc_feature 1..21: note = Description of Artificial Sequence: Synthetic oligonucleotide
  misc_difference 20..21: note = any ribo-nucleotide or 2'-deoxy-nucleotide, 2'-OMe-U, or a modified or chemically modified nucleotide; note = one or more of the "n" positions may be absent, and if absent, the terminal residue may be connected to an inverted nucleotide via a 3' to 3' bond
  source 1..21: mol_type = other RNA
acagcagggtctcaaaaggnn

SEQ ID NO: 196 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
atagttggtgtagatgaggtt

SEQ ID NO: 197 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 2: mod_base = OTHER; note = 2'-deoxy-nucleotide (2'-deoxyuridine)
  modified_base 5..6: mod_base = OTHER; note = 2'-deoxy-nucleotide (2'-deoxyuridine)
  source 1..21: mol_type = other DNA
  misc_feature 1..8: note = DNA
  misc_feature 9..21: note = RNA
atagttggtgtagatgaggtt

SEQ ID NO: 198 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 2: mod_base = OTHER; note = 2'-deoxy-nucleotide (2'-deoxyuridine)
  modified_base 5..6: mod_base = OTHER; note = 2'-deoxy-nucleotide (2'-deoxyuridine)
  source 1..21: mol_type = other DNA
  misc_feature 1: note = RNA
  misc_feature 1: note = RNA
  misc_feature 2..8: note = DNA
  misc_feature 9..21: note = RNA
atagttggtgtagatgaggtt

SEQ ID NO: 199 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 5..6: mod_base = OTHER; note = 2'-deoxy-nucleotide (2'-deoxyuridine)
  source 1..21: mol_type = other DNA
  misc_feature 1..2: note = RNA
  misc_feature 3..8: note = DNA
  misc_feature 9..21: note = RNA
atagttggtgtagatgaggtt

SEQ ID NO: 200 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 5..6: mod_base = OTHER; note = 2'-deoxy-nucleotide (2'-deoxyuridine)
  source 1..21: mol_type = other DNA
  misc_feature 1..3: note = RNA
  misc_feature 4..8: note = DNA
  misc_feature 9..21: note = RNA
atagttggtgtagatgaggtt

SEQ ID NO: 201 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 5..6: mod_base = OTHER; note = 2'-deoxy-nucleotide (2'-deoxyuridine)
  source 1..21: mol_type = other DNA
  misc_feature 1..4: note = RNA
  misc_feature 5..8: note = DNA
  misc_feature 9..21: note = RNA
atagttggtgtagatgaggtt

SEQ ID NO: 202 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 5: mod_base = OTHER; note = 2'-deoxy-nucleotide (2'-deoxyuridine)
  misc_feature 6: note = RNA
  misc_feature 2: note = RNA
  source 1..21: mol_type = other DNA
  misc_feature 4: note = RNA
  misc_feature 1: note = DNA
  misc_feature 2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
atagttggtgtagatgaggtt

SEQ ID NO: 203 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 5: mod_base = OTHER; note = 2'-deoxy-nucleotide (2'-deoxyuridine)
  misc_feature 6: note = RNA
  source 1..21: mol_type = other DNA
  misc_feature 4: note = RNA
  misc_feature 1..2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
atagttggtgtagatgaggtt

SEQ ID NO: 204 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 2: mod_base = OTHER; note = 2'-deoxy-nucleotide (2'-deoxyuridine)
  misc_feature 5: note = RNA
  modified_base 6: mod_base = OTHER; note = 2'-deoxy-nucleotide (2'-deoxyuridine)
  misc_feature 1: note = RNA
  source 1..21: mol_type = other DNA
  misc_feature 3: note = RNA
  misc_feature 7: note = RNA
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
atagttggtgtagatgaggtt

SEQ ID NO: 205 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  misc_feature 5: note = RNA
  modified_base 6: mod_base = OTHER; note = 2'-deoxy-nucleotide (2'-deoxyuridine)
  misc_feature 11: note = RNA
  source 1..21: mol_type = other DNA
  misc_feature 7: note = RNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
atagttggtgtagatgaggtt

SEQ ID NO: 206 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  misc_feature 1: note = RNA
  modified_base 14: mod_base = OTHER; note = 2'-OMe-nucleotide
  modified_base 16: mod_base = OTHER; note = 2'-OMe-nucleotide
  modified_base 18: mod_base = OTHER; note = 2'-OMe-nucleotide
  modified_base 20..21: mod_base = OTHER; note = 2'-OMe-nucleotide
  source 1..21: mol_type = other DNA
  modified_base 2: mod_base = OTHER; note = 2'-deoxy-nucleotide (2'-deoxyuridine)
  modified_base 5..6: mod_base = OTHER; note = 2'-deoxy-nucleotide (2'-deoxyuridine)
  misc_feature 1: note = RNA
  misc_feature 2..8: note = DNA
  misc_feature 9..21: note = RNA
atagttggtgtagatgaggtt

SEQ ID NO: 207 - RNA, 21 nt, synthetic construct
  misc_feature 1..21: note = Description of Artificial Sequence: Synthetic oligonucleotide
  misc_difference 20..21: note = one or more of the "n" positions may be absent, and if absent, the terminal residue may be connected to an inverted nucleotide via a 3' to 3' bond; note = any ribo-nucleotide or 2'-deoxy-nucleotide, 2'-OMe-U, or a modified or chemically modified nucleotide
  source 1..21: mol_type = other RNA
gatgactatgtgaaggcacnn

SEQ ID NO: 208 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ggatgactatgtgaaggcatt

SEQ ID NO: 209 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ggatgactatgtgaaggcatt

SEQ ID NO: 210 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ggatgactatgtgaaggcatt

SEQ ID NO: 211 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ggatgactatgtgaaggcatt

SEQ ID NO: 212 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ggatgactatgtgaaggcatt

SEQ ID NO: 213 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ggatgactatgtgaaggcatt

SEQ ID NO: 214 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ggatgactatgtgaaggcatt

SEQ ID NO: 215 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ggatgactatgtgaaggcatt

SEQ ID NO: 216 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ggatgactatgtgaaggcatt

SEQ ID NO: 217 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ggatgactatgtgaaggcatt

SEQ ID NO: 218 - RNA, 21 nt, synthetic construct
  misc_feature 1..21: note = Description of Artificial Sequence: Synthetic oligonucleotide
  modified_base 1: mod_base = OTHER; note = 2'-OMe-nucleotide
  modified_base 3: mod_base = OTHER; note = 2'-OMe-nucleotide
  modified_base 5: mod_base = OTHER; note = 2'-OMe-nucleotide
  modified_base 7: mod_base = OTHER; note = 2'-OMe-nucleotide
  modified_base 20..21: mod_base = OTHER; note = 2'-OMe-nucleotide
  source 1..21: mol_type = other RNA
ggatgactatgtgaaggcatt

SEQ ID NO: 219 - RNA, 21 nt, synthetic construct
  misc_feature 1..21: note = Description of Artificial Sequence: Synthetic oligonucleotide
  modified_base 11: mod_base = OTHER; note = 2'-deoxy-2'-fluoro-nucleotide
  modified_base 13: mod_base = OTHER; note = 2'-deoxy-2'-fluoro-nucleotide
  modified_base 20..21: mod_base = OTHER; note = 2'-OMe-nucleotide
  source 1..21: mol_type = other RNA
ggatgactatgtgaaggcatt

SEQ ID NO: 220 - RNA, 21 nt, synthetic construct
  misc_feature 1..21: note = Description of Artificial Sequence: Synthetic oligonucleotide
  modified_base 1..2: mod_base = OTHER; note = 2'-OMe-nucleotide
  modified_base 20..21: mod_base = OTHER; note = 2'-OMe-nucleotide
  source 1..21: mol_type = other RNA
ggatgactatgtgaaggcatt

SEQ ID NO: 221 - RNA, 21 nt, synthetic construct
  misc_feature 1..21: note = Description of Artificial Sequence: Synthetic oligonucleotide
  modified_base 1..2: mod_base = OTHER; note = 2'-OMe-nucleotide
  modified_base 20..21: mod_base = OTHER; note = 2'-OMe-nucleotide
  source 1..21: mol_type = other RNA
ggatgactatgtgaaggcatt

SEQ ID NO: 222 - RNA, 21 nt, synthetic construct
  misc_feature 1..21: note = Description of Artificial Sequence: Synthetic oligonucleotide
  misc_difference 20..21: note = one or more of the "n" positions may be absent, and if absent, the terminal residue may be connected to an inverted nucleotide via a 3' to 3' bond; note = any ribo-nucleotide or 2'-deoxy-nucleotide, 2'-OMe-U, or a modified or chemically modified nucleotide
  source 1..21: mol_type = other RNA
gtgccttcacatagtcatcnn

SEQ ID NO: 223 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
tgccttcacatagtcatcctt

SEQ ID NO: 224 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 1: mod_base = OTHER; note = 2'-deoxy-nucleotide (2'-deoxyuridine)
  modified_base 5..6: mod_base = OTHER; note = 2'-deoxy-nucleotide (2'-deoxyuridine)
  source 1..21: mol_type = other DNA
  misc_feature 1..8: note = DNA
  misc_feature 9..21: note = RNA
tgccttcacatagtcatcctt

SEQ ID NO: 225 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  misc_feature 1: note = RNA
  modified_base 5..6: mod_base = OTHER; note = 2'-deoxy-nucleotide (2'-deoxyuridine)
  source 1..21: mol_type = other DNA
  misc_feature 1: note = RNA
  misc_feature 2..8: note = DNA
  misc_feature 9..21: note = RNA
tgccttcacatagtcatcctt

SEQ ID NO: 226 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 5..6: mod_base = OTHER; note = 2'-deoxy-nucleotide (2'-deoxyuridine)
  source 1..21: mol_type = other DNA
  misc_feature 1..2: note = RNA
  misc_feature 3..8: note = DNA
  misc_feature 9..21: note = RNA
tgccttcacatagtcatcctt

SEQ ID NO: 227 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 5..6: mod_base = OTHER; note = 2'-deoxy-nucleotide (2'-deoxyuridine)
  source 1..21: mol_type = other DNA
  misc_feature 1..3: note = RNA
  misc_feature 4..8: note = DNA
  misc_feature 9..21: note = RNA
tgccttcacatagtcatcctt

SEQ ID NO: 228 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 5..6: mod_base = OTHER; note = 2'-deoxy-nucleotide (2'-deoxyuridine)
  source 1..21: mol_type = other DNA
  misc_feature 1..4: note = RNA
  misc_feature 5..8: note = DNA
  misc_feature 9..21: note = RNA
tgccttcacatagtcatcctt

SEQ ID NO: 229 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 1: mod_base = OTHER; note = 2'-deoxy-nucleotide (2'-deoxyuridine)
  modified_base 5: mod_base = OTHER; note = 2'-deoxy-nucleotide (2'-deoxyuridine)
  misc_feature 6: note = RNA
  misc_feature 17: note = RNA
  source 1..21: mol_type = other DNA
  misc_feature 2: note = RNA
  misc_feature 4: note = RNA
  misc_feature 1: note = DNA
  misc_feature 2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
tgccttcacatagtcatcctt

SEQ ID NO: 230 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 5: mod_base = OTHER; note = 2'-deoxy-nucleotide (2'-deoxyuridine)
  misc_feature 6: note = RNA
  source 1..21: mol_type = other DNA
  misc_feature 4: note = RNA
  misc_feature 1..2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
tgccttcacatagtcatcctt

SEQ ID NO: 231 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  misc_feature 1: note = RNA
  misc_feature 5: note = RNA
  modified_base 6: mod_base = OTHER; note = 2'-deoxy-nucleotide (2'-deoxyuridine)
  source 1..21: mol_type = other DNA
  misc_feature 3: note = RNA
  misc_feature 7: note = RNA
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
tgccttcacatagtcatcctt

SEQ ID NO: 232 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  misc_feature 5: note = RNA
  modified_base 6: mod_base = OTHER; note = 2'-deoxy-nucleotide (2'-deoxyuridine)
  source 1..21: mol_type = other DNA
  misc_feature 7: note = RNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
tgccttcacatagtcatcctt

SEQ ID NO: 233 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  misc_feature 1: note = RNA
  modified_base 5..6: mod_base = OTHER; note = 2'-deoxy-nucleotide (2'-deoxyuridine)
  modified_base 14: mod_base = OTHER; note = 2'-OMe-nucleotide
  modified_base 16: mod_base = OTHER; note = 2'-OMe-nucleotide
  modified_base 18: mod_base = OTHER; note = 2'-OMe-nucleotide
  modified_base 20..21: mod_base = OTHER; note = RNA 2'-OMe-nucleotide
  source 1..21: mol_type = other DNA
  misc_feature 1: note = RNA
  misc_feature 2..8: note = DNA
  misc_feature 9..21: note = RNA
tgccttcacatagtcatcctt

SEQ ID NO: 234 - RNA, 21 nt, synthetic construct
  misc_feature 1..21: note = Description of Artificial Sequence: Synthetic oligonucleotide
  modified_base 4: mod_base = OTHER; note = 2'-deoxy-2'-fluoro-nucleotide
  modified_base 20..21: mod_base = OTHER; note = 2'-OMe-nucleotide
  source 1..21: mol_type = other RNA
tgccttcacatagtcatcctt

SEQ ID NO: 235 - RNA, 21 nt, synthetic construct
  misc_feature 1..21: note = Description of Artificial Sequence: Synthetic oligonucleotide
  modified_base 9: mod_base = OTHER; note = 2'-OMe-nucleotide
  modified_base 11: mod_base = OTHER; note = 2'-OMe-nucleotide
  modified_base 13: mod_base = OTHER; note = 2'-OMe-nucleotide
  modified_base 15: mod_base = OTHER; note = 2'-OMe-nucleotide
  modified_base 17: mod_base = OTHER; note = 2'-OMe-nucleotide
  modified_base 19..21: mod_base = OTHER; note = 2'-OMe-nucleotide
  source 1..21: mol_type = other RNA
tgccttcacatagtcatcctt

SEQ ID NO: 236 - RNA, 21 nt, synthetic construct
  misc_feature 1..21: note = Description of Artificial Sequence: Synthetic oligonucleotide
  modified_base 11: mod_base = OTHER; note = 2'-OMe-nucleotide
  modified_base 13: mod_base = OTHER; note = 2'-OMe-nucleotide
  modified_base 15: mod_base = OTHER; note = 2'-OMe-nucleotide
  modified_base 17: mod_base = OTHER; note = 2'-OMe-nucleotide
  modified_base 19..21: mod_base = OTHER; note = 2'-OMe-nucleotide
  source 1..21: mol_type = other RNA
tgccttcacatagtcatcctt

SEQ ID NO: 237 - RNA, 21 nt, synthetic construct
  misc_feature 1..21: note = Description of Artificial Sequence: Synthetic oligonucleotide
  misc_difference 20..21: note = one or more of the "n" positions may be absent, and if absent, the terminal residue may be connected to an inverted nucleotide via a 3' to 3' bond; note = any ribo-nucleotide or 2'-deoxy-nucleotide, 2'-OMe-U, or a modified or chemically modified nucleotide
  source 1..21: mol_type = other RNA
gaagccttttgagaccctgnn

SEQ ID NO: 238 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctgtt

SEQ ID NO: 239 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctgtt

SEQ ID NO: 240 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctgtt

SEQ ID NO: 241 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctgtt

SEQ ID NO: 242 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctgtt

SEQ ID NO: 243 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctgtt

SEQ ID NO: 244 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctgtt

SEQ ID NO: 245 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctgtt

SEQ ID NO: 246 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctgtt

SEQ ID NO: 247 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
gaagccttttgagaccctgtt

SEQ ID NO: 248 - RNA, 21 nt, synthetic construct
  misc_feature 1..21: note = Description of Artificial Sequence: Synthetic oligonucleotide
  modified_base 1: mod_base = OTHER; note = 2'-OMe-nucleotide
  modified_base 3: mod_base = OTHER; note = 2'-OMe-nucleotide
  modified_base 5: mod_base = OTHER; note = 2'-OMe-nucleotide
  modified_base 7: mod_base = OTHER; note = 2'-OMe-nucleotide
  modified_base 9: mod_base = OTHER; note = 2'-OMe-nucleotide
  modified_base 20..21: mod_base = OTHER; note = 2'-OMe-nucleotide
  source 1..21: mol_type = other RNA
gaagccttttgagaccctgtt

SEQ ID NO: 249 - RNA, 21 nt, synthetic construct
  misc_feature 1..21: note = Description of Artificial Sequence: Synthetic oligonucleotide
  misc_difference 20..21: note = any ribo-nucleotide or 2'-deoxy-nucleotide, 2'-OMe-U, or a modified or chemically modified nucleotide; note = one or more of the "n" positions may be absent, and if absent, the terminal residue may be connected to an inverted nucleotide via a 3' to 3' bond
  source 1..21: mol_type = other RNA
cagggtctcaaaaggcttcnn

SEQ ID NO: 250 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cagggtctcaaaaggcttctt

SEQ ID NO: 251 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 6: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  modified_base 8: mod_base = OTHER; note = 2'-deoxy-nucleotide (2'-deoxyuridine)
  misc_feature 1..8: note = DNA
  misc_feature 9..21: note = RNA
cagggtctcaaaaggcttctt

SEQ ID NO: 252 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 6: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  misc_feature 1: note = RNA
  modified_base 8: mod_base = OTHER; note = 2'-deoxy-nucleotide (2'-deoxyuridine)
  misc_feature 1: note = RNA
  misc_feature 2..8: note = DNA
  misc_feature 9..21: note = RNA
cagggtctcaaaaggcttctt

SEQ ID NO: 253 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 6: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  modified_base 8: mod_base = OTHER; note = 2'-deoxy-nucleotide (2'-deoxyuridine)
  misc_feature 1..2: note = RNA
  misc_feature 3..8: note = DNA
  misc_feature 9..21: note = RNA
cagggtctcaaaaggcttctt

SEQ ID NO: 254 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 6: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  modified_base 8: mod_base = OTHER; note = 2'-deoxy-nucleotide (2'-deoxyuridine)
  misc_feature 1..3: note = RNA
  misc_feature 4..8: note = DNA
  misc_feature 9..21: note = RNA
cagggtctcaaaaggcttctt

SEQ ID NO: 255 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 6: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  misc_structure 9..21: note = RNA
  modified_base 8: mod_base = OTHER; note = 2'-deoxy-nucleotide (2'-deoxyuridine)
  misc_feature 1..4: note = RNA
  misc_feature 5..8: note = DNA
  misc_feature 9..21: note = RNA
cagggtctcaaaaggcttctt

SEQ ID NO: 256 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  source 1..21: mol_type = other DNA
  misc_feature 2: note = RNA
  misc_feature 4: note = RNA
  misc_feature 6: note = RNA
  misc_feature 1: note = DNA
  misc_feature 2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
cagggtctcaaaaggcttctt

SEQ ID NO: 257 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  misc_feature 6: note = RNA
  source 1..21: mol_type = other DNA
  misc_feature 4: note = RNA
  misc_feature 1..2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
cagggtctcaaaaggcttctt

SEQ ID NO: 258 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 6: mod_base = OTHER; note = 2'-deoxy-nucleotide (2'-deoxyuridine)
  modified_base 8: mod_base = OTHER; note = 2'-deoxy-nucleotide (2'-deoxyuridine)
  source 1..21: mol_type = other DNA
  misc_feature 1: note = RNA
  misc_feature 3: note = RNA
  misc_feature 5: note = RNA
  misc_feature 7: note = RNA
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
cagggtctcaaaaggcttctt

SEQ ID NO: 259 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 20..21: mod_base = OTHER; note = 2'-OMe-nucleotide
  modified_base 6: mod_base = OTHER; note = 2'-deoxy-nucleotide (2'-deoxyuridine)
  modified_base 8: mod_base = OTHER; note = 2'-deoxy-nucleotide (2'-deoxyuridine)
  source 1..21: mol_type = other DNA
  misc_feature 5: note = RNA
  misc_feature 7: note = RNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
cagggtctcaaaaggcttctt

SEQ ID NO: 260 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 8: mod_base = OTHER; note = 2'-deoxy-nucleotide (2'-deoxyuridine)
  modified_base 14: mod_base = OTHER; note = 2'-OMe-nucleotide
  modified_base 16: mod_base = OTHER; note = 2'-OMe-nucleotide
  modified_base 18: mod_base = OTHER; note = 2'-OMe-nucleotide
  modified_base 20..21: mod_base = OTHER; note = 2'-OMe-nucleotide
  source 1..21: mol_type = other DNA
  misc_feature 1: note = RNA
  modified_base 6: mod_base = OTHER; note = 2'-deoxy-nucleotide (2'-deoxyuridine)
  misc_feature 1: note = RNA
  misc_feature 2..8: note = DNA
  misc_feature 9..21: note = RNA
cagggtctcaaaaggcttctt

SEQ ID NO: 261 - RNA, 21 nt, synthetic construct
  misc_feature 1..21: note = Description of Artificial Sequence: Synthetic oligonucleotide
  misc_difference 20..21: note = one or more of the "n" positions may be absent, and if absent, the terminal residue may be connected to an inverted nucleotide via a 3' to 3' bond; note = any ribo-nucleotide or 2'-deoxy-nucleotide, 2'-OMe-U, or a modified or chemically modified nucleotide
  source 1..21: mol_type = other RNA
cctcatctacaccaactatnn

SEQ ID NO: 262 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 263 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 264 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 265 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 266 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 267 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 268 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 269 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 270 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 271 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
cctcatctacaccaactattt

SEQ ID NO: 272 - RNA, 21 nt, synthetic construct
  misc_feature 1..21: note = Description of Artificial Sequence: Synthetic oligonucleotide
  modified_base 1: mod_base = OTHER; note = 2'-OMe-nucleotide
  modified_base 3: mod_base = OTHER; note = 2'-OMe-nucleotide
  modified_base 5: mod_base = OTHER; note = 2'-OMe-nucleotide
  modified_base 7: mod_base = OTHER; note = 2'-OMe-nucleotide
  modified_base 9: mod_base = OTHER; note = 2'-OMe-nucleotide
  modified_base 20..21: mod_base = OTHER; note = 2'-OMe-nucleotide
  source 1..21: mol_type = other RNA
cctcatctacaccaactattt

SEQ ID NO: 273 - RNA, 21 nt, synthetic construct
  misc_feature 1..21: note = Description of Artificial Sequence: Synthetic oligonucleotide
  misc_difference 20..21: note = any ribo-nucleotide or 2'-deoxy-nucleotide, 2'-OMe-U, or a modified or chemically modified nucleotide; note = one or more of the "n" positions may be absent, and if absent, the terminal residue may be connected to an inverted nucleotide via a 3' to 3' bond
  source 1..21: mol_type = other RNA
atagttggtgtagatgaggnn

SEQ ID NO: 274 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
atagttggtgtagatgaggtt

SEQ ID NO: 275 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 2: mod_base = OTHER; note = 2'-deoxy-nucleotide (2'-deoxyuridine)
  modified_base 5..6: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  misc_feature 1..8: note = DNA
  misc_feature 9..21: note = RNA
atagttggtgtagatgaggtt

SEQ ID NO: 276 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 2: mod_base = OTHER; note = 2'-deoxy-nucleotide (2'-deoxyuridine)
  modified_base 5..6: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  misc_feature 1: note = RNA
  misc_feature 1: note = RNA
  misc_feature 2..8: note = DNA
  misc_feature 9..21: note = RNA
atagttggtgtagatgaggtt

SEQ ID NO: 277 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 5..6: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  misc_feature 1..2: note = RNA
  misc_feature 3..8: note = DNA
  misc_feature 9..21: note = RNA
atagttggtgtagatgaggtt

SEQ ID NO: 278 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 5..6: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  misc_feature 1..3: note = RNA
  misc_feature 4..8: note = DNA
  misc_feature 9..21: note = RNA
atagttggtgtagatgaggtt

SEQ ID NO: 279 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 5..6: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  misc_feature 1..4: note = RNA
  misc_feature 5..8: note = DNA
  misc_feature 9..21: note = RNA
atagttggtgtagatgaggtt

SEQ ID NO: 280 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  misc_feature 2: note = RNA
  modified_base 5: mod_base = OTHER; note = 2'-deoxy-nucleotide (2'-deoxyuridine)
  misc_feature 6: note = RNA
  modified_base 20..21: mod_base = OTHER; note = 2'-OMe-nucleotide
  source 1..21: mol_type = other DNA
  misc_feature 4: note = RNA
  misc_feature 1: note = DNA
  misc_feature 2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
atagttggtgtagatgaggtt

SEQ ID NO: 281 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 5: mod_base = OTHER; note = 2'-deoxy-nucleotide (2'-deoxyuridine)
  misc_feature 6: note = RNA
  misc_feature 4: note = RNA
  misc_feature 15: note = RNA
  modified_base 20..21: mod_base = OTHER; note = 2'-OMe-nucleotide
  source 1..21: mol_type = other DNA
  misc_feature 1..2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
atagttggtgtagatgaggtt

SEQ ID NO: 282 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 2: mod_base = OTHER; note = 2'-deoxy-nucleotide (2'-deoxyuridine)
  misc_feature 5: note = RNA
  modified_base 6: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  misc_feature 1: note = RNA
  misc_feature 3: note = RNA
  misc_feature 7: note = RNA
  misc_feature 1: note = RNA
  misc_feature 2: note = DNA
  misc_feature 3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
atagttggtgtagatgaggtt

SEQ ID NO: 283 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  misc_feature 5: note = RNA
  modified_base 6: mod_base = OTHER; note = 2'-deoxy-nucleotide (2'-deoxyuridine)
  misc_feature 11: note = RNA
  misc_feature 7: note = RNA
  modified_base 20..21: mod_base = OTHER; note = 2'-OMe-nucleotide
  source 1..21: mol_type = other DNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
atagttggtgtagatgaggtt

SEQ ID NO: 284 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 2: mod_base = OTHER; note = 2'-deoxy-nucleotide (2'-deoxyuridine)
  modified_base 5..6: mod_base = OTHER; note = 2'-deoxy-nucleotide (2'-deoxyuridine)
  misc_feature 1: note = RNA
  modified_base 14: mod_base = OTHER; note = 2'-OMe-nucleotide
  modified_base 16: mod_base = OTHER; note = 2'-OMe-nucleotide
  modified_base 18: mod_base = OTHER; note = 2'-OMe-nucleotide
  modified_base 20..21: mod_base = OTHER; note = 2'-OMe-nucleotide
  source 1..21: mol_type = other DNA
  misc_feature 1: note = RNA
  misc_feature 2..8: note = DNA
  misc_feature 9..21: note = RNA
atagttggtgtagatgaggtt

SEQ ID NO: 285 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
cttagtgactttacttgtatt

SEQ ID NO: 286 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
cagaccagcatgacagatttt

SEQ ID NO: 287 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
tgatcttctccaagaggaatt

SEQ ID NO: 288 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gttcattgcactttgattatt

SEQ ID NO: 289 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
cattgcactttgattagcatt

SEQ ID NO: 290 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
agcgatggaacttcgactttt

SEQ ID NO: 291 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gcgatggaacttcgacttttt

SEQ ID NO: 292 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gggaagggacacacaagaatt

SEQ ID NO: 293 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
tctacctcaggcagctcaatt

SEQ ID NO: 294 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
ggtgctcaataaatgattctt

SEQ ID NO: 295 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
catcatcaaaaactttggatt

SEQ ID NO: 296 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
aaggagtcagacattttaatt

SEQ ID NO: 297 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gtgctgggcatttttattttt

SEQ ID NO: 298 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gccggcttcatgccagctatt

SEQ ID NO: 299 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gggcatcatcaaaaacttttt

SEQ ID NO: 300 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gaagggcaccctagttctatt

SEQ ID NO: 301 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
cagttcattgcactttgattt

SEQ ID NO: 302 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
acaaggagtcagacatttttt

SEQ ID NO: 303 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
tggaggcactgaagtgctttt

SEQ ID NO: 304 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gcagggaccacaccctgtatt

SEQ ID NO: 305 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
ctgtactgttctgtgtctttt

SEQ ID NO: 306 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
ttaaacacctcctcatgtatt

SEQ ID NO: 307 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
agactctcagggtcgaaaatt

SEQ ID NO: 308 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
catgacagatttctaccactt

SEQ ID NO: 309 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
agatttctaccactccaaatt

SEQ ID NO: 310 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
ccaagaggaagccctaatctt

SEQ ID NO: 311 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gacagcagaggaagaccattt

SEQ ID NO: 312 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
ctcccacaatgctgaatattt

SEQ ID NO: 313 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
tacaagtaaagtcactaagtt

SEQ ID NO: 314 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
aatctgtcatgctggtctgtt

SEQ ID NO: 315 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
ttcctcttggagaagatcatt

SEQ ID NO: 316 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
taatcaaagtgcaatgaactt

SEQ ID NO: 317 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
tgctaatcaaagtgcaatgtt

SEQ ID NO: 318 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
aagtcgaagttccatcgcttt

SEQ ID NO: 319 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
aaagtcgaagttccatcgctt

SEQ ID NO: 320 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
ttcttgtgtgtcccttccctt

SEQ ID NO: 321 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
ttgagctgcctgaggtagatt

SEQ ID NO: 322 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gaatcatttattgagcacctt

SEQ ID NO: 323 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
tccaaagtttttgatgatgtt

SEQ ID NO: 324 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
ttaaaatgtctgactcctttt

SEQ ID NO: 325 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
aaataaaaatgcccagcactt

SEQ ID NO: 326 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
tagctggcatgaagccggctt

SEQ ID NO: 327 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
aaagtttttgatgatgccctt

SEQ ID NO: 328 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
tagaactagggtgcccttctt

SEQ ID NO: 329 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
atcaaagtgcaatgaactgtt

SEQ ID NO: 330 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
aaaatgtctgactccttgttt

SEQ ID NO: 331 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
aagcacttcagtgcctccatt

SEQ ID NO: 332 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
tacagggtgtggtccctgctt

SEQ ID NO: 333 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
aagacacagaacagtacagtt

SEQ ID NO: 334 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
tacatgaggaggtgtttaatt

SEQ ID NO: 335 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
ttttcgaccctgagagtcttt

SEQ ID NO: 336 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gtggtagaaatctgtcatgtt

SEQ ID NO: 337 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
tttggagtggtagaaatcttt

SEQ ID NO: 338 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
gattagggcttcctcttggtt

SEQ ID NO: 339 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
atggtcttcctctgctgtctt

SEQ ID NO: 340 - RNA, 21 nt, synthetic construct
  misc_feature 1..21: note = Description of Artificial Sequence: Synthetic oligonucleotide
  modified_base 20..21: mod_base = OTHER; note = 2'-methoxy-nucleotide
  source 1..21: mol_type = other RNA
atattcagcattgtgggagtt

SEQ ID NO: 341 - RNA, 21 nt, synthetic construct
  misc_feature 1..21: note = Description of Artificial Sequence: Synthetic oligonucleotide
  misc_difference 20..21: note = any ribo-nucleotide or 2'-deoxy-nucleotide, 2'-OMe-U, or a modified or chemically modified nucleotide; note = one or more of the "n" positions may be absent, and if absent, the terminal residue may be connected to an inverted nucleotide via a 3' to 3' bond
  source 1..21: mol_type = other RNA
aaggagtcagacattttaann

SEQ ID NO: 342 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 8: mod_base = OTHER; note = 2'-OMe-nucleotide
  modified_base 12: mod_base = OTHER; note = 2'-OMe-nucleotide
  modified_base 17: mod_base = OTHER; note = 2'-OMe-nucleotide
  modified_base 20..21: mod_base = OTHER; note = 2'-OMe-nucleotide
  source 1..21: mol_type = other RNA
aaggagtcagacattttaatt

SEQ ID NO: 343 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 8: mod_base = OTHER; note = 2'-OMe-nucleotide
  modified_base 12: mod_base = OTHER; note = 2'-OMe-nucleotide
  modified_base 14..15: mod_base = OTHER; note = 2'-OMe-nucleotide
  modified_base 17: mod_base = OTHER; note = 2'-OMe-nucleotide
  modified_base 20..21: mod_base = OTHER; note = 2'-OMe-nucleotide
  source 1..21: mol_type = other RNA
aaggagtcagacattttaatt

SEQ ID NO: 344 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 8: mod_base = OTHER; note = 2'-OMe-nucleotide
  modified_base 12: mod_base = OTHER; note = 2'-OMe-nucleotide
  modified_base 14..15: mod_base = OTHER; note = 2'-OMe-nucleotide
  modified_base 17: mod_base = OTHER; note = 2'-OMe-nucleotide
  modified_base 20..21: mod_base = OTHER; note = 2'-OMe-nucleotide
  source 1..21: mol_type = other RNA
aaggagtcagacattttaatt

SEQ ID NO: 345 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 8: mod_base = OTHER; note = 2'-OMe-nucleotide
  modified_base 12: mod_base = OTHER; note = 2'-OMe-nucleotide
  modified_base 17: mod_base = OTHER; note = 2'-OMe-nucleotide
  modified_base 20..21: mod_base = OTHER; note = 2'-OMe-nucleotide
  source 1..21: mol_type = other RNA
aaggagtcagacattttaatt

SEQ ID NO: 346 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
aaggagtcagacattttaatt

SEQ ID NO: 347 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
aaggagtcagacattttaatt

SEQ ID NO: 348 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
aaggagtcagacattttaatt

SEQ ID NO: 349 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
aaggagtcagacattttaatt

SEQ ID NO: 350 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
aaggagtcagacattttaatt

SEQ ID NO: 351 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
aaggagtcagacattttaatt

SEQ ID NO: 352 - RNA, 21 nt, synthetic construct
  misc_feature 1..21: note = Description of Artificial Sequence: Synthetic oligonucleotide
  modified_base 8: mod_base = OTHER; note = 2'-OMe-nucleotide
  modified_base 12: mod_base = OTHER; note = 2'-OMe-nucleotide
  modified_base 17: mod_base = OTHER; note = 2'-OMe-nucleotide
  modified_base 20..21: mod_base = OTHER; note = 2'-OMe-nucleotide
  source 1..21: mol_type = other RNA
aaggagtcagacattttaatt

SEQ ID NO: 353 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 20..21: mod_base = OTHER; note = 2'-OMe-nucleotide
  source 1..21: mol_type = other RNA
aaggagtcagacattttaatt

SEQ ID NO: 354 - RNA, 21 nt, synthetic construct
  misc_feature 1..21: note = Description of Artificial Sequence: Synthetic oligonucleotide
  modified_base 1..3: mod_base = OTHER; note = 2'-OMe-nucleotide
  modified_base 8: mod_base = OTHER; note = 2'-OMe-nucleotide
  modified_base 12: mod_base = OTHER; note = 2'-OMe-nucleotide
  modified_base 17: mod_base = OTHER; note = 2'-OMe-nucleotide
  modified_base 20..21: mod_base = OTHER; note = 2'-OMe-nucleotide
  source 1..21: mol_type = other RNA
aaggagtcagacattttaatt

SEQ ID NO: 355 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  misc_difference 20..21: note = ribo-nucleotide or 2'-deoxy-nucleotide, 2'-OMe-U, or a modified, inverted or chemically modified nucleotide; note = a, c, t, g, u, unknown or other
  source 1..21: mol_type = other DNA
  misc_feature 1..19: note = RNA
ttaaaatgtctgactccttnn

SEQ ID NO: 356 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 2: mod_base = OTHER; note = 2'-OMe-nucleotide
  modified_base 7: mod_base = OTHER; note = 2'-OMe-nucleotide
  modified_base 11: mod_base = OTHER; note = 2'-OMe-nucleotide
  modified_base 20..21: mod_base = OTHER; note = 2'-OMe-nucleotide
  source 1..21: mol_type = other DNA
  misc_feature 3: note = RNA
  misc_feature 5: note = RNA
  misc_feature 7: note = RNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
ttaaaatgtctgactcctttt

SEQ ID NO: 357 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 2: mod_base = OTHER; note = 2'-OMe-nucleotide
  modified_base 7: mod_base = OTHER; note = 2'-OMe-nucleotide
  modified_base 11: mod_base = OTHER; note = 2'-OMe-nucleotide
  modified_base 20..21: mod_base = OTHER; note = 2'-OMe-nucleotide
  source 1..21: mol_type = other DNA
  misc_feature 5: note = RNA
  misc_feature 7: note = RNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
ttaaaatgtctgactcctttt

SEQ ID NO: 358 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 2: mod_base = OTHER; note = 2'-OMe-nucleotide
  misc_feature 7: note = RNA
  misc_feature 5: note = RNA
  modified_base 20..21: mod_base = OTHER; note = 2'-OMe-nucleotide
  source 1..21: mol_type = other DNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
ttaaaatgtctgactcctttt

SEQ ID NO: 359 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 2: mod_base = OTHER; note = 2'-OMe-nucleotide
  misc_feature 7: note = RNA
  misc_feature 5: note = RNA
  modified_base 20..21: mod_base = OTHER; note = 2'-OMe-nucleotide
  source 1..21: mol_type = other DNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
ttaaaatgtctgactcctttt

SEQ ID NO: 360 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 7: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  misc_feature 1..2: note = RNA
  misc_feature 3..8: note = DNA
  misc_feature 9..21: note = RNA
ttaaaatgtctgactcctttt

SEQ ID NO: 361 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 7: mod_base = OTHER; note = 2'-deoxy-nucleotide (2'-deoxyuridine)
  modified_base 20..21: mod_base = OTHER; note = 2'-OMe-nucleotide
  source 1..21: mol_type = other DNA
  misc_feature 1..4: note = RNA
  misc_feature 5..8: note = DNA
  misc_feature 9..21: note = RNA
ttaaaatgtctgactcctttt

SEQ ID NO: 362 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 1: mod_base = OTHER; note = 2'-deoxy-nucleotide (2'-deoxyuridine)
  misc_feature 2: note = RNA
  modified_base 7: mod_base = OTHER; note = 2'-deoxy-nucleotide (2'-deoxyuridine)
  misc_feature 4: note = RNA
  misc_feature 6: note = RNA
  modified_base 20..21: mod_base = OTHER; note = 2'-OMe-nucleotide
  source 1..21: mol_type = other DNA
  misc_feature 1: note = DNA
  misc_feature 2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
ttaaaatgtctgactcctttt

SEQ ID NO: 363 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 7: mod_base = OTHER; note = 2'-deoxy-nucleotide (2'-deoxyuridine)
  misc_feature 6: note = RNA
  misc_feature 4: note = RNA
  modified_base 20..21: mod_base = OTHER; note = 2'-OMe-nucleotide
  source 1..21: mol_type = other DNA
  misc_feature 1..2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
ttaaaatgtctgactcctttt

SEQ ID NO: 364 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  misc_feature 5: note = RNA
  misc_feature 7: note = RNA
  modified_base 20..21: mod_base = OTHER; note = 2'-OMe-nucleotide
  source 1..21: mol_type = other DNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
ttaaaatgtctgactcctttt

SEQ ID NO: 365 - RNA, 21 nt, synthetic construct
  misc_feature 1..21: note = Description of Artificial Sequence: Synthetic oligonucleotide
  modified_base 2: mod_base = OTHER; note = 2'-OMe-nucleotide
  modified_base 7: mod_base = OTHER; note = 2'-OMe-nucleotide
  modified_base 11: mod_base = OTHER; note = 2'-OMe-nucleotide
  modified_base 20..21: mod_base = OTHER; note = 2'-OMe-nucleotide
  source 1..21: mol_type = other RNA
ttaaaatgtctgactcctttt

SEQ ID NO: 366 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ttaaaatgtctgactcctttt

SEQ ID NO: 367 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ttaaaatgtctgactcctttt

SEQ ID NO: 368 - RNA, 21 nt, synthetic construct
  misc_feature 1..21: note = Description of Artificial Sequence: Synthetic oligonucleotide
  modified_base 20..21: mod_base = OTHER; note = 2'-OMe-nucleotide
  source 1..21: mol_type = other RNA
ttaaaatgtctgactcctttt

SEQ ID NO: 369 - RNA, 21 nt, synthetic construct
  misc_feature 1..21: note = Description of Artificial Sequence: Synthetic oligonucleotide
  source 1..21: mol_type = other RNA
cgagaacagtttgtacaagtt

SEQ ID NO: 370 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
caggcctctacaactactatt

SEQ ID NO: 371 - RNA, 25 nt, synthetic construct
  misc_feature 1..25: note = Description of Artificial Sequence: Synthetic oligonucleotide
  source 1..25: mol_type = other RNA
gagcactccaagatcaacttccgcg

SEQ ID NO: 372 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ggacaggcctctacaactatt

SEQ ID NO: 373 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gagcactccaagatcaacttt

SEQ ID NO: 374 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
gaacactccaagatcaacttt

SEQ ID NO: 375 - RNA, 25 nt, synthetic construct
  misc_feature 1..25: note = Description of Artificial Sequence: Synthetic oligonucleotide
  source 1..25: mol_type = other RNA
caggcctctacaactactacgacga

SEQ ID NO: 376 - RNA, 25 nt, synthetic construct
  misc_feature 1..25: note = Description of Artificial Sequence: Synthetic oligonucleotide
  source 1..25: mol_type = other RNA
gaacactccaagatcaacttccgag

SEQ ID NO: 377 - RNA, 25 nt, synthetic construct
  misc_feature 1..25: note = Description of Artificial Sequence: Synthetic oligonucleotide
  source 1..25: mol_type = other RNA
ggacaggcctctacaactactacga

SEQ ID NO: 378 - DNA, 26 bp, synthetic construct
  misc_feature 1..26: note = Description of Artificial Sequence: Synthetic oligonucleotide
  misc_feature 1..26: note = Description of Combined DNA/RNA Molecule: Synthetic oligonucleotide
  source 1..26: mol_type = other DNA
  misc_feature 1..19: note = RNA
  misc_feature 20..26: note = DNA
caggcctctacaactactattaaaaa

SEQ ID NO: 379 - RNA, 19 nt, synthetic construct
  misc_feature 1..19: note = Description of Artificial Sequence: Synthetic oligonucleotide
  source 1..19: mol_type = other RNA
caggcctctacaactacta

SEQ ID NO: 380 - DNA, 33 bp, synthetic construct
  misc_feature 1..33: note = Description of Artificial Sequence: Synthetic oligonucleotide
  misc_feature 1..33: note = Description of Combined DNA/RNA Molecule: Synthetic oligonucleotide
  source 1..33: mol_type = other DNA
  misc_feature 1..19: note = RNA
  misc_feature 20..33: note = DNA
caggcctctacaactactattaaaaaaaaaaaa

SEQ ID NO: 381 - DNA, 26 bp, synthetic construct
  misc_feature 1..26: note = Description of Artificial Sequence: Synthetic oligonucleotide
  misc_feature 1..26: note = Description of Combined DNA/RNA Molecule: Synthetic oligonucleotide
  source 1..26: mol_type = other DNA
  misc_feature 1..5: note = DNA
  misc_feature 6..24: note = RNA
  misc_feature 25..26: note = DNA
aaaaacaggcctctacaactactatt

SEQ ID NO: 382 - DNA, 29 bp, synthetic construct
  misc_feature 1..29: note = Description of Artificial Sequence: Synthetic oligonucleotide
  misc_feature 1..29: note = Description of Combined DNA/RNA Molecule: Synthetic oligonucleotide
  source 1..29: mol_type = other DNA
  misc_feature 1..19: note = RNA
  misc_feature 20..29: note = DNA
caggcctctacaactactattaaaaaaaa

SEQ ID NO: 383 - DNA, 29 bp, synthetic construct
  misc_feature 1..29: note = Description of Artificial Sequence: Synthetic oligonucleotide
  misc_feature 1..29: note = Description of Combined DNA/RNA Molecule: Synthetic oligonucleotide
  source 1..29: mol_type = other DNA
  misc_feature 1..8: note = DNA
  misc_feature 9..27: note = RNA
  misc_feature 28..29: note = DNA
aaaaaaaacaggcctctacaactactatt

SEQ ID NO: 384 - DNA, 33 bp, synthetic construct; identical to SEQ ID NO: 380
  misc_feature 1..33: note = Description of Artificial Sequence: Synthetic oligonucleotide
  misc_feature 1..33: note = Description of Combined DNA/RNA Molecule: Synthetic oligonucleotide
  source 1..33: mol_type = other DNA
  misc_feature 1..19: note = RNA
  misc_feature 20..33: note = DNA

SEQ ID NO: 385 - DNA, 37 bp, synthetic construct
  misc_feature 1..37: note = Description of Artificial Sequence: Synthetic oligonucleotide
  misc_feature 1..37: note = Description of Combined DNA/RNA Molecule: Synthetic oligonucleotide
  source 1..37: mol_type = other DNA
  misc_feature 1..16: note = DNA
  misc_feature 17..35: note = RNA
  misc_feature 36..37: note = DNA
aaaaaaaaaaaaaaaacaggcctctacaactactatt

SEQ ID NO: 386 - DNA, 37 bp, synthetic construct
  misc_feature 1..37: note = Description of Artificial Sequence: Synthetic oligonucleotide
  misc_feature 1..37: note = Description of Combined DNA/RNA Molecule: Synthetic oligonucleotide
  source 1..37: mol_type = other DNA
  misc_feature 1..19: note = RNA
  misc_feature 20..37: note = DNA
caggcctctacaactactattaaaaaaaaaaaaaaaa

SEQ ID NO: 387 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
caggcctctacaactactatt

SEQ ID NO: 388 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ggacaggcctgtacaactatt

SEQ ID NO: 389 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
ggacaggcctctacaactatt

SEQ ID NO: 390 - RNA, 21 nt, synthetic construct
  misc_feature 1..21: note = Description of Artificial Sequence: Synthetic oligonucleotide
  source 1..21: mol_type = other RNA
cttgtacaaactgttctcgtt

SEQ ID NO: 391 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagtagttgtagaggcctgtt

SEQ ID NO: 392 - RNA, 27 nt, synthetic construct
  misc_feature 1..27: note = Description of Artificial Sequence: Synthetic oligonucleotide
  source 1..27: mol_type = other RNA
cgcggaagttgatcttggagtgctctt

SEQ ID NO: 393 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagttgtagaggcctgtcctt

SEQ ID NO: 394 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
agttgatcttggagtgctctt

SEQ ID NO: 395 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
agttgatcttggagtgttctt

SEQ ID NO: 396 - RNA, 27 nt, synthetic construct
  misc_feature 1..27: note = Description of Artificial Sequence: Synthetic oligonucleotide
  source 1..27: mol_type = other RNA
tcgtcgtagtagttgtagaggcctgtt

SEQ ID NO: 397 - RNA, 27 nt, synthetic construct
  misc_feature 1..27: note = Description of Artificial Sequence: Synthetic oligonucleotide
  source 1..27: mol_type = other RNA
ctcggaagttgatcttggagtgttctt

SEQ ID NO: 398 - RNA, 27 nt, synthetic construct
  misc_feature 1..27: note = Description of Artificial Sequence: Synthetic oligonucleotide
  source 1..27: mol_type = other RNA
tcgtagtagttgtagaggcctgtcctt

SEQ ID NO: 399 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagtagttgtagaggcctgtt

SEQ ID NO: 400 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagtagttgtagaggcctgtt

SEQ ID NO: 401 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagtagttgtagaggcctgtt

SEQ ID NO: 402 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagtagttgtagaggcctgtt

SEQ ID NO: 403 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagtagttgtagaggcctgtt

SEQ ID NO: 404 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagtagttgtagaggcctgtt

SEQ ID NO: 405 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagtagttgtagaggcctgtt

SEQ ID NO: 406 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagtagttgtagaggcctgtt

SEQ ID NO: 407 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagtagttgtagaggcctgtt

SEQ ID NO: 408 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagtagttgtagaggcctgtt

SEQ ID NO: 409 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagttgtacaggcctgtcctt

SEQ ID NO: 410 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 1..19: note = RNA
  misc_feature 20..21: note = DNA
tagttgtagaggcctgtcctt

SEQ ID NO: 411 - RNA, 19 nt, synthetic construct
  misc_feature 1..19: note = Description of Artificial Sequence: Synthetic oligonucleotide
  misc_feature 1: note = 5'-Inverted 1,2-dideoxy-D-Ribose-linked
  misc_feature 15..19: note = 2'-5' linkage between nucleotides
  misc_feature 19: note = 3'-C3 spacer-linked
  source 1..19: mol_type = other RNA
actccaagatcaacttcct

SEQ ID NO: 412 - RNA, 19 nt, synthetic construct
  misc_feature 1..19: note = Description of Artificial Sequence: Synthetic oligonucleotide
  modified_base 2: mod_base = OTHER; note = 2'-O-Methyl ribonucleotide
  modified_base 4: mod_base = OTHER; note = 2'-O-Methyl ribonucleotide
  modified_base 6: mod_base = OTHER; note = 2'-O-Methyl ribonucleotide
  misc_feature 7..8: note = 2'-5' linkage between nucleotides
  modified_base 8: mod_base = OTHER; note = 2'-O-Methyl ribonucleotide
  modified_base 11: mod_base = OTHER; note = 2'-O-Methyl ribonucleotide
  modified_base 13: mod_base = OTHER; note = 2'-O-Methyl ribonucleotide
  modified_base 15: mod_base = OTHER; note = 2'-O-Methyl ribonucleotide
  modified_base 17: mod_base = OTHER; note = 2'-O-Methyl ribonucleotide
  modified_base 19: mod_base = OTHER; note = 2'-O-Methyl ribonucleotide
  misc_feature 19: note = 3'-C3-C3 spacer-linked
  source 1..19: mol_type = other RNA
aggaagttgatcttggagt

SEQ ID NO: 413 - RNA, 19 nt, synthetic construct
  misc_feature 1..19: note = Description of Artificial Sequence: Synthetic oligonucleotide
  misc_feature 1: note = 5'-Inverted 1,2-dideoxy-D-Ribose-linked
  modified_base 4: mod_base = OTHER; note = 2'-O-Methyl ribonucleotide
  misc_feature 9..10: note = 2'-5' linkage between nucleotides
  modified_base 11: mod_base = OTHER; note = 2'-O-Methyl ribonucleotide
  modified_base 13: mod_base = OTHER; note = 2'-O-Methyl ribonucleotide
  modified_base 17: mod_base = OTHER; note = 2'-O-Methyl ribonucleotide
  misc_feature 19: note = 3'-C3 spacer-linked
  source 1..19: mol_type = other RNA
tcctgagacacatgggtga

SEQ ID NO: 414 - RNA, 19 nt, synthetic construct
  misc_feature 1..19: note = Description of Artificial Sequence: Synthetic oligonucleotide
  modified_base 1: mod_base = OTHER; note = 2'-O-Methyl ribonucleotide
  modified_base 4: mod_base = OTHER; note = 2'-O-Methyl ribonucleotide
  misc_feature 6..7: note = 2'-5' linkage between nucleotides
  modified_base 8: mod_base = OTHER; note = 2'-O-Methyl ribonucleotide
  modified_base 11: mod_base = OTHER; note = 2'-O-Methyl ribonucleotide
  modified_base 15: mod_base = OTHER; note = 2'-O-Methyl ribonucleotide
  misc_feature 19: note = 3'-C3-C3 spacer-linked
  source 1..19: mol_type = other RNA
tcacccatgtgtctcagga

SEQ ID NO: 415 - RNA, 19 nt, synthetic construct
  misc_feature 1..19: note = Description of Artificial Sequence: Synthetic oligonucleotide
  misc_feature 1: note = 5'-Inverted 1,2-dideoxy-D-Ribose-linked
  misc_feature 15..19: note = 2'-5' linkage between nucleotides
  misc_feature 19: note = 3'-C3 spacer-phosphate-linked
  source 1..19: mol_type = other RNA
gagacacatgggtgctata

SEQ ID NO: 416 - RNA, 19 nt, synthetic construct
  misc_feature 1..19: note = Description of Artificial Sequence: Synthetic oligonucleotide
  modified_base 1: mod_base = OTHER; note = 2'-O-Methyl ribonucleotide
  modified_base 3: mod_base = OTHER; note = 2'-O-Methyl ribonucleotide
  modified_base 5: mod_base = OTHER; note = 2'-O-Methyl ribonucleotide
  misc_feature 7..8: note = 2'-5' linkage between nucleotides
  modified_base 9: mod_base = OTHER; note = 2'-O-Methyl ribonucleotide
  modified_base 11: mod_base = OTHER; note = 2'-O-Methyl ribonucleotide
  modified_base 13: mod_base = OTHER; note = 2'-O-Methyl ribonucleotide
  modified_base 15: mod_base = OTHER; note = 2'-O-Methyl ribonucleotide
  modified_base 17: mod_base = OTHER; note = 2'-O-Methyl ribonucleotide
  modified_base 19: mod_base = OTHER; note = 2'-O-Methyl ribonucleotide
  misc_feature 19: note = 3'-C3-C3 spacer-linked
  source 1..19: mol_type = other RNA
tatagcacccatgtgtctc

SEQ ID NO: 417 - RNA, 19 nt, synthetic construct
  misc_feature 1..19: note = Description of Artificial Sequence: Synthetic oligonucleotide
  misc_feature 1: note = 5'-Inverted 1,2-dideoxy-D-Ribose-linked
  modified_base 2..3: mod_base = OTHER; note = 2'-O-Methyl ribonucleotide
  modified_base 5: mod_base = OTHER; note = 2'-O-Methyl ribonucleotide
  modified_base 7..8: mod_base = OTHER; note = 2'-O-Methyl ribonucleotide
  misc_feature 9..10: note = 2'-5' linkage between nucleotides
  modified_base 12: mod_base = OTHER; note = 2'-O-Methyl ribonucleotide
  modified_base 14..15: mod_base = OTHER; note = 2'-O-Methyl ribonucleotide
  modified_base 17: mod_base = OTHER; note = 2'-O-Methyl ribonucleotide
  misc_feature 19: note = 3'-C3 spacer-linked
  source 1..19: mol_type = other RNA
cttacgctgagtacttcgt

SEQ ID NO: 418 - RNA, 19 nt, synthetic construct
  misc_feature 1..19: note = Description of Artificial Sequence: Synthetic oligonucleotide
  modified_base 2: mod_base = OTHER; note = 2'-O-Methyl ribonucleotide
  misc_feature 6..7: note = 2'-5' linkage between nucleotides
  modified_base 7: mod_base = OTHER; note = 2'-O-Methyl ribonucleotide
  modified_base 11: mod_base = OTHER; note = 2'-O-Methyl ribonucleotide
  modified_base 16: mod_base = OTHER; note = 2'-O-Methyl ribonucleotide
  misc_feature 19: note = 3'-C3-C3 spacer-linked
  source 1..19: mol_type = other RNA
acgaagtactcagcgtaag

SEQ ID NO: 419 - RNA, 21 nt, synthetic construct
  misc_feature 1..21: note = Description of Artificial Sequence: Synthetic oligonucleotide
  source 1..21: mol_type = other RNA
gccttccaaggatgggtttgt

SEQ ID NO: 420 - RNA, 21 nt, synthetic construct
  misc_feature 1..21: note = Description of Artificial Sequence: Synthetic oligonucleotide
  source 1..21: mol_type = other RNA
ggagttcttccatgtagagga

SEQ ID NO: 421 - RNA, 21 nt, synthetic construct
  misc_feature 1..21: note = Description of Artificial Sequence: Synthetic oligonucleotide
  source 1..21: mol_type = other RNA
ccatgtagaggacctagaagg

SEQ ID NO: 422 - RNA, 21 nt, synthetic construct
  misc_feature 1..21: note = Description of Artificial Sequence: Synthetic oligonucleotide
  source 1..21: mol_type = other RNA
gccttccaaggatgggtttgt

SEQ ID NO: 423 - RNA, 21 nt, synthetic construct
  misc_feature 1..21: note = Description of Artificial Sequence: Synthetic oligonucleotide
  source 1..21: mol_type = other RNA
gccttccaaggatgggtttgt

SEQ ID NO: 424 - RNA, 21 nt, synthetic construct
  misc_feature 1..21: note = Description of Artificial Sequence: Synthetic oligonucleotide
  source 1..21: mol_type = other RNA
gccttccaaggatgggtttgt

SEQ ID NO: 425 - RNA, 21 nt, synthetic construct
  misc_feature 1..21: note = Description of Artificial Sequence: Synthetic oligonucleotide
  source 1..21: mol_type = other RNA
gccttccaaggatgggtttgt

SEQ ID NO: 426 - RNA, 21 nt, synthetic construct
  misc_feature 1..21: note = Description of Artificial Sequence: Synthetic oligonucleotide
  source 1..21: mol_type = other RNA
gccttccaaggatgggtttgt

SEQ ID NO: 427 - RNA, 21 nt, synthetic construct
  misc_feature 1..21: note = Description of Artificial Sequence: Synthetic oligonucleotide
  source 1..21: mol_type = other RNA
aaacccatccttggaaggccg

SEQ ID NO: 428 - RNA, 21 nt, synthetic construct
  misc_feature 1..21: note = Description of Artificial Sequence: Synthetic oligonucleotide
  source 1..21: mol_type = other RNA
ctctacatggaagaactccac

SEQ ID NO: 429 - RNA, 21 nt, synthetic construct
  misc_feature 1..21: note = Description of Artificial Sequence: Synthetic oligonucleotide
  source 1..21: mol_type = other RNA
ttctaggtcctctacatggaa

SEQ ID NO: 430 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 8: mod_base = OTHER; note = 2'-deoxy-nucleotide (2'-deoxyuridine)
  source 1..21: mol_type = other DNA
  misc_feature 5: note = RNA
  misc_feature 7: note = RNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
aaacccatccttggaaggccg

SEQ ID NO: 431 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 4: note = RNA
  misc_feature 6: note = RNA
  misc_feature 1..2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
aaacccatccttggaaggccg

SEQ ID NO: 432 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 2: note = RNA
  misc_feature 4: note = RNA
  misc_feature 6: note = RNA
  misc_feature 1: note = DNA
  misc_feature 2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
aaacccatccttggaaggccg

SEQ ID NO: 433 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 8: mod_base = OTHER; note = 2'-deoxy-nucleotide (2'-deoxyuridine)
  source 1..21: mol_type = other DNA
  misc_feature 1..2: note = RNA
  misc_feature 3..8: note = DNA
  misc_feature 9..21: note = RNA
aaacccatccttggaaggccg

SEQ ID NO: 434 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 8: mod_base = OTHER; note = 2'-deoxy-nucleotide (2'-deoxyuridine)
  source 1..21: mol_type = other DNA
  misc_feature 1..4: note = RNA
  misc_feature 5..8: note = DNA
  misc_feature 9..21: note = RNA
aaacccatccttggaaggccg

SEQ ID NO: 435 - RNA, 21 nt, synthetic construct
  misc_feature 1..21: note = Description of Artificial Sequence: Synthetic oligonucleotide
  source 1..21: mol_type = other RNA
gctcattgtcgaggtccttga

SEQ ID NO: 436 - RNA, 21 nt, synthetic construct
  misc_feature 1..21: note = Description of Artificial Sequence: Synthetic oligonucleotide
  source 1..21: mol_type = other RNA
gccaaacctgtggctacaagt

SEQ ID NO: 437 - RNA, 21 nt, synthetic construct
  misc_feature 1..21: note = Description of Artificial Sequence: Synthetic oligonucleotide
  source 1..21: mol_type = other RNA
ggaagacgcgacatgtcaaca

SEQ ID NO: 438 - RNA, 21 nt, synthetic construct
  misc_feature 1..21: note = Description of Artificial Sequence: Synthetic oligonucleotide
  source 1..21: mol_type = other RNA
gctcattgtcgaggtccttga

SEQ ID NO: 439 - RNA, 21 nt, synthetic construct
  misc_feature 1..21: note = Description of Artificial Sequence: Synthetic oligonucleotide
  source 1..21: mol_type = other RNA
gctcattgtcgaggtccttga

SEQ ID NO: 440 - RNA, 21 nt, synthetic construct
  misc_feature 1..21: note = Description of Artificial Sequence: Synthetic oligonucleotide
  source 1..21: mol_type = other RNA
gctcattgtcgaggtccttga

SEQ ID NO: 441 - RNA, 21 nt, synthetic construct
  misc_feature 1..21: note = Description of Artificial Sequence: Synthetic oligonucleotide
  source 1..21: mol_type = other RNA
gctcattgtcgaggtccttga

SEQ ID NO: 442 - RNA, 21 nt, synthetic construct
  misc_feature 1..21: note = Description of Artificial Sequence: Synthetic oligonucleotide
  source 1..21: mol_type = other RNA
gctcattgtcgaggtccttga

SEQ ID NO: 443 - RNA, 21 nt, synthetic construct
  misc_feature 1..21: note = Description of Artificial Sequence: Synthetic oligonucleotide
  source 1..21: mol_type = other RNA
aaggacctcgacaatgagctc

SEQ ID NO: 444 - RNA, 21 nt, synthetic construct
  misc_feature 1..21: note = Description of Artificial Sequence: Synthetic oligonucleotide
  source 1..21: mol_type = other RNA
ttgtagccacaggtttggcaa

SEQ ID NO: 445 - RNA, 21 nt, synthetic construct
  misc_feature 1..21: note = Description of Artificial Sequence: Synthetic oligonucleotide
  source 1..21: mol_type = other RNA
ttgacatgtcgcgtcttcctg

SEQ ID NO: 446 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 8: mod_base = OTHER; note = 2'-deoxy-nucleotide (2'-deoxyuridine)
  source 1..21: mol_type = other DNA
  misc_feature 5: note = RNA
  misc_feature 7: note = RNA
  misc_feature 1..3: note = RNA
  misc_feature 4: note = DNA
  misc_feature 5: note = RNA
  misc_feature 6: note = DNA
  misc_feature 7: note = RNA
  misc_feature 8: note = DNA
  misc_feature 9..21: note = RNA
aaggacctcgacaatgagctc

SEQ ID NO: 447 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 4: note = RNA
  misc_feature 6: note = RNA
  misc_feature 1..2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
aaggacctcgacaatgagctc

SEQ ID NO: 448 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  source 1..21: mol_type = other DNA
  misc_feature 2: note = RNA
  misc_feature 4: note = RNA
  misc_feature 6: note = RNA
  misc_feature 1: note = DNA
  misc_feature 2: note = RNA
  misc_feature 3: note = DNA
  misc_feature 4: note = RNA
  misc_feature 5: note = DNA
  misc_feature 6: note = RNA
  misc_feature 7: note = DNA
  misc_feature 8..21: note = RNA
aaggacctcgacaatgagctc

SEQ ID NO: 449 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 8: mod_base = OTHER; note = 2'-deoxy-nucleotide (2'-deoxyuridine)
  source 1..21: mol_type = other DNA
  misc_feature 1..2: note = RNA
  misc_feature 3..8: note = DNA
  misc_feature 9..21: note = RNA
aaggacctcgacaatgagctc

SEQ ID NO: 450 - DNA, 21 bp, synthetic construct
  misc_feature 1..21: note = Description of Artificial Sequence: Synthetic oligonucleotide
  misc_feature 1..21: note = Description of Combined DNA/RNA Molecule: Synthetic oligonucleotide
  modified_base 8: mod_base = OTHER; note = 2'-deoxy-nucleotide (2'-deoxyuridine)
  source 1..21: mol_type = other DNA
  misc_feature 1..4: note = RNA
  misc_feature 5..8: note = DNA
  misc_feature 9..21: note = RNA
aaggacctcgacaatgagctc

SEQ ID NO: 451 - DNA, 50 bp, synthetic construct
  misc_feature 1..50: note = Description of Artificial Sequence: Synthetic oligonucleotide
  misc_feature 1..50: note = Description of Combined DNA/RNA Molecule: Synthetic oligonucleotide
  source 1..50: mol_type = other DNA
  misc_feature 1..13: note = DNA
  misc_feature 14..33: note = RNA
  misc_feature 34: note = DNA
  misc_feature 35: note = RNA
  misc_feature 36: note = DNA
  misc_feature 37: note = RNA
  misc_feature 38..50: note = DNA
  modified_base 14: mod_base = OTHER; note = thymine
  modified_base 21..24: mod_base = OTHER; note = thymine
  modified_base 32: mod_base = OTHER; note = thymine
  modified_base 35: mod_base = OTHER; note = thymine
  modified_base 37: mod_base = OTHER; note = thymine
ctcgaggggcaactgaagccttttgagaccctgctgtcccaggcggccgc

SEQ ID NO: 452 - DNA, 50 bp, synthetic construct
  misc_feature 1..50: note = Description of Artificial Sequence: Synthetic oligonucleotide
  misc_feature 1..50: note = Description of Combined DNA/RNA Molecule: Synthetic oligonucleotide
  source 1..50: mol_type = other DNA
  misc_feature 1..7: note = DNA
  misc_feature 8: note = RNA
  misc_feature 9..15: note = DNA
  misc_feature 16..34: note = RNA
  misc_feature 35..36: note = DNA
  misc_feature 37..38: note = RNA
  misc_feature 39..50: note = DNA
  modified_base 8: mod_base = OTHER; note = thymine
  modified_base 21: mod_base = OTHER; note = thymine
  modified_base 23: mod_base = OTHER; note = thymine
  modified_base 32..33: mod_base = OTHER; note = thymine
  modified_base 37..38: mod_base = OTHER; note = thymine
ctcgagctgggacagcagggtctcaaaaggcttcagttgcccgcggccgc

SEQ ID NO: 453 - DNA, 50 bp, synthetic construct
  misc_feature 1..50: note = Description of Artificial Sequence: Synthetic oligonucleotide
  misc_feature 1..50: note = Description of Combined DNA/RNA Molecule: Synthetic oligonucleotide
  source 1..50: mol_type = other DNA
  misc_feature 1..13: note = DNA
  misc_feature 14..33: note = RNA
  misc_feature 34: note = DNA
  misc_feature 35: note = RNA
  misc_feature 36: note = DNA
  misc_feature 37: note = RNA
  misc_feature 38..50: note = DNA
  modified_base 14: mod_base = OTHER; note = thymine
  modified_base 16: mod_base = OTHER; note = thymine
  modified_base 32: mod_base = OTHER; note = thymine
  modified_base 35: mod_base = OTHER; note = thymine
  modified_base 37: mod_base = OTHER; note = thymine
ctcgaggggcaactctacgcaaaacagaccctgctgtcccaggcggccgc

SEQ ID NO: 454 - DNA, 71 bp, synthetic construct
  misc_feature 1..71: note = Description of Artificial Sequence: Synthetic oligonucleotide
  misc_feature 1..71: note = Description of Combined DNA/RNA Molecule: Synthetic oligonucleotide
  source 1..71: mol_type = other DNA
  misc_feature 1..13: note = DNA
  misc_feature 14..33: note = RNA
  misc_feature 34: note = DNA
  misc_feature 35..54: note = RNA
  misc_feature 55: note = DNA
  misc_feature 56: note = RNA
  misc_feature 57: note = DNA
  misc_feature 58: note = RNA
  misc_feature 59..71: note = DNA
  modified_base 14: mod_base = OTHER; note = thymine
  modified_base 16: mod_base = OTHER; note = thymine
  modified_base 32: mod_base = OTHER; note = thymine
  modified_base 35: mod_base = OTHER; note = thymine
  modified_base 37: mod_base = OTHER; note = thymine
  modified_base 53: mod_base = OTHER; note = thymine
  modified_base 56: mod_base = OTHER; note = thymine
  modified_base 58: mod_base = OTHER; note = thymine
ctcgaggggcaactctacgcaaaacagaccctgctctacgcaaaacagaccctgctgtcccaggcggccgc

SEQ ID NO: 455 - DNA, 92 bp, synthetic construct
  misc_feature 1..92: note = Description of Artificial Sequence: Synthetic oligonucleotide
  misc_feature 1..92: note = Description of Combined DNA/RNA Molecule: Synthetic oligonucleotide
  source 1..92: mol_type = other DNA
  misc_feature 1..13: note = DNA
  misc_feature 14..33: note = RNA
  misc_feature 34: note = DNA
  misc_feature 35..54: note = RNA
  misc_feature 55: note = DNA
  misc_feature 56..75: note = RNA
  misc_feature 76: note = DNA
  misc_feature 77: note = RNA
  misc_feature 78: note = DNA
  misc_feature 79: note = RNA
  misc_feature 80..92: note = DNA
  modified_base 14: mod_base = OTHER; note = thymine
  modified_base 16: mod_base = OTHER; note = thymine
  modified_base 32: mod_base = OTHER; note = thymine
  modified_base 35: mod_base = OTHER; note = thymine
  modified_base 37: mod_base = OTHER; note = thymine
  modified_base 53: mod_base = OTHER; note = thymine
  modified_base 56: mod_base = OTHER; note = thymine
  modified_base 58: mod_base = OTHER; note = thymine
  modified_base 74: mod_base = OTHER; note = thymine
  modified_base 77: mod_base = OTHER; note = thymine
  modified_base 79: mod_base = OTHER; note = thymine
ctcgaggggcaactctacgcaaaacagaccctgctctacgcaaaacagaccctgctctacgcaaaacagaccctgctgtcccaggcggccgc

SEQ ID NO: 456 - DNA, 113 bp, synthetic construct
  misc_feature 1..113: note = Description of Artificial Sequence: Synthetic oligonucleotide
  misc_feature 1..113: note = Description of Combined DNA/RNA Molecule: Synthetic oligonucleotide
  source 1..113: mol_type = other DNA
  misc_feature 1..13: note = DNA
  misc_feature 14..33: note = RNA
  misc_feature 34: note = DNA
  misc_feature 35..54: note = RNA
  misc_feature 55: note = DNA
  misc_feature 56..75: note = RNA
  misc_feature 76: note = DNA
  misc_feature 77..96: note = RNA
  misc_feature 97: note = DNA
  misc_feature 98: note = RNA
  misc_feature 99: note = DNA
  misc_feature 100: note = RNA
  misc_feature 101..113: note = DNA
  modified_base 14: mod_base = OTHER; note = thymine
  modified_base 16: mod_base = OTHER; note = thymine
  modified_base 32: mod_base = OTHER; note = thymine
  modified_base 35: mod_base = OTHER; note = thymine
  modified_base 37: mod_base = OTHER; note = thymine
  modified_base 53: mod_base = OTHER; note = thymine
  modified_base 56: mod_base = OTHER; note = thymine
  modified_base 58: mod_base = OTHER; note = thymine
  modified_base 74: mod_base = OTHER; note = thymine
  modified_base 77: mod_base = OTHER; note = thymine
  modified_base 79: mod_base = OTHER; note = thymine
  modified_base 95: mod_base = OTHER; note = thymine
  modified_base 98: mod_base = OTHER; note = thymine
  modified_base 100: mod_base = OTHER; note = thymine
ctcgaggggcaactctacgcaaaacagaccctgctctacgcaaaacagaccctgctctacgcaaaacagaccctgctctacgcaaaacagaccctgctgtcccaggcggccgc

(19) United States
(12) Patent Application Publication
(10) Pub. No.: US 2026/0250686 A1
(43) Pub. Date: Aug. 27, 2026

(54) SIRNA STRUCTURES FOR HIGH ACTIVITY AND REDUCED OFF TARGET

(71) Applicant: Nitto Denko Corporation, Osaka (JP)

(72) Inventors: Kenjirou Minomi, Osaka (JP); Jens Harborth, San Diego, CA (US); Cima Cina, San Diego, CA (US); Wenbin Ying, San Diego, CA (US); Jane Zheng, San Diego, CA (US); Narendra Vaish, Kirkland, WA (US)

(21) Appl. No.: 19/537,345

(22) Filed: Feb. 11, 2026

Related U.S. Application Data
(63) Continuation of application No. 18/598,943, filed on Mar. 7, 2024, now abandoned.
Application No. 18/598,943 is a continuation of application No. 17/661,803, filed on May 3, 2022, now Pat. No. 11,926,831.
Application No. 17/661,803 is a continuation of application No. 16/451,757, filed on Jun. 25, 2019, now Pat. No. 11,390,871.
Application No. 16/451,757 is a continuation of application No. 15/376,633, filed on Dec. 12, 2016, now Pat. No. 10,358,647.
(60) Provisional application No. 62/266,675, filed on Dec. 13, 2015.

(51) Int. Cl. C12N 15/113

(52) U.S. Cl. CPC C12N 15/1137; C12Y 205/01018; C12N 2310/14; C12N 2310/321; C12N 2310/322; C12N 2310/343

(57) ABSTRACT

Compounds, compositions, and methods for modulating expression of target genes using RNA interference. RNAi structures and molecules can be used for modulating or silencing the expression of genes, with high levels of RNAi activity and reduced off target actions. Advantageous structures include siRNAs targeted to any gene having one or more 2′-deoxy nucleotides located in the seed region. The RNA interference molecules can be used in methods for preventing or treating diseases.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### SEQUENCE LISTING IN ELECTRONIC FORMAT

The Electronic Sequence Listing in ST.26 format is submitted in the present application. The Electronic Sequence Listing is provided as a file entitled SL_HRAK001_001C4.xml created and last saved on Feb. 10, 2026, which is approximately 1280 bytes in size. The information in the Electronic Sequence Listing is incorporated herein by reference in its entirety.

### BACKGROUND

RNA interference (RNAi) has been used for sequence-specific post-transcriptional gene silencing in animals mediated by short interfering RNAs (siRNAs). See, e.g., Zamore et al., Cell, 2000, Vol. 101, pp. 25-33; Fire et al., Nature, 1998, Vol. 391, pp. 806811; Sharp, Genes & Development, 1999, Vol. 13, pp. 139-141.

An RNAi response in cells can be triggered by a double stranded RNA (dsRNA), although the mechanism is not yet fully understood. In general, siRNAs can be from about 21 to about 23 nucleotides in length and include a base pair duplex region about 19 nucleotides in length.

RNAi involves an endonuclease complex known as the RNA induced silencing complex (RISC). An siRNA has an antisense or guide strand which enters the RISC complex and mediates cleavage of a single stranded RNA target having a sequence complementary to the antisense strand of the siRNA duplex. The other strand of the siRNA is the passenger strand. Cleavage of the target RNA takes place in the middle of the region complementary to the antisense strand of the siRNA duplex See, e.g., Elbashir et al., Genes & Development, 2001, Vol. 15, pp. 188-200.

To improve the properties of siRNA compounds for use as drug agents, the structure of siRNA compounds has been modified in various ways to improve stability and reduce off target effects. Off target activity of an siRNA involves modulation of cellular nucleic acids other than the targeted RNA, which can occur through various mechanisms.

Modifications of siRNA compounds include structural variation of the base, sugar, and/or backbone of any of the nucleotides. One way to modify siRNA nucleotides is to replace certain ribonucleotides in the siRNA with deoxynucleotides. In particular, deoxynucleotides are often utilized in the overhang or terminal nucleotides of the siRNA structure.

However, a drawback of using deoxynucleotides in an siRNA structure is that the deoxynucleotides cannot be used in the seed region of the siRNA. This is because such modification of the siRNA is not favorable because gene silencing activity is reduced.

There is an urgent need for RNAi structures for modulating the expression of genes that reduce off target action without loss of activity.

In particular, therapeutics based on RNAi suppression of oncogenes and cancer-related genes will require highly potent and stable siRNA sequences and structures.

What is needed are siRNA compounds with highly active structure for modulating gene expression.

### SUMMARY

This invention relates to the fields of biopharmaceuticals and therapeutics composed of nucleic acid based molecules. More particularly, this invention relates to the structures of compounds utilizing RNA interference (RNAi) for modulating the expression of genes, and uses thereof.

This invention relates to compounds, compositions and methods for modulating the expression of target genes using RNA interference. The siRNA compounds and structures of this disclosure can be highly active for modulating gene expression.

The RNAi structures of this invention can be used for modulating the expression of genes with surprisingly high levels of activity and reduced off target actions.

In some embodiments, this invention provides molecules for gene silencing by RNA interference that reduce off target action, without loss of activity.

In further embodiments, this invention provides molecules for gene silencing by RNA interference that reduce off target action, with unexpectedly high gene silencing activity.

The RNAi molecules of this invention can provide highly potent and stable siRNA structures that can be used for therapeutics based on RNAi suppression of genes.

In further embodiments, the structures, molecules and compositions of this invention can be used in methods for preventing or treating diseases, or ameliorating symptoms of conditions or disorders associated with one or more genes.

Embodiments of this invention include the following:

A nucleic acid molecule, wherein:
a) the molecule has a polynucleotide sense strand and a polynucleotide antisense strand;
b) each strand of the molecule is from 15 to 30 nucleotides in length;
c) a contiguous region of from 15 to 30 nucleotides of the antisense strand is complementary to a sequence of a mRNA;
d) at least a portion of the sense strand is complementary to at least a portion of the antisense strand, and the molecule has a duplex region of from 15 to 30 nucleotides in length, wherein one or more of the nucleotides in the duplex region at positions 3 to 8 from the 5′ end of the antisense strand are deoxynucleotides. The mRNA can be a human mRNA.

In some embodiments, the nucleic acid molecule antisense strand can have deoxynucleotides in a plurality of positions, the plurality of positions being one of the following:
each of positions 4, 6 and 8, from the 5′ end of the antisense strand;
each of positions 3, 5 and 7, from the 5′ end of the antisense strand;
each of positions 1, 3, 5 and 7, from the 5′ end of the antisense strand;
each of positions 3-8, from the 5′ end of the antisense strand; and
each of positions 5-8, from the 5′ end of the antisense strand.

The nucleic acid molecules can be RNAi molecules that are active for modulating expression of the mRNA.

In certain embodiments, the nucleic acid molecules can be active for inhibiting expression of a gene selected from protein coding genes, proto-oncogenes, oncogenes, tumor suppressor genes, and cell signaling genes.

In additional embodiments, the mRNA can be a human mRNA expressing any member or sub-member of the human family of proteins including SRY, beta-globin, RAS, cytosolic GST, mitochondrial GST, MAPEG GST, GST-π, p16, p21, p53, serum albumin, Type VII collagen, Complement C3, Apolipoprotein B, phenylalanine hydroxylase, Factor VIII, Huntingtin, RB1 retinoblastoma protein, CFTR, Titin, Utrophin, and Dystrophin.

The nucleic acid molecules can have an IC50 for knockdown of the mRNA of less than 100 μM, or less than 50 μM, or less than 10 μM. In certain embodiments, the nucleic acid molecules can inhibit the mRNA by at least 25% in vivo after a single administration of the molecules.

In some embodiments, each strand of the molecule is from 18 to 22 nucleotides in length. The duplex region can be 19 nucleotides in length. In certain embodiments, the polynucleotide sense strand and the polynucleotide antisense strand can be connected as a single strand, and form a duplex region connected at one end by a loop.

The nucleic acid molecules can have a blunt end. In certain embodiments, the nucleic acid molecules can have one or more 3′ overhangs.

Embodiments of this invention include nucleic acid molecules having one or more of the nucleotides in the duplex region being modified or chemically-modified. The modified or chemically-modified nucleotides can be 2′-O-alkyl substituted nucleotides, 2′-deoxy-2′-fluoro substituted nucleotides, phosphorothioate nucleotides, locked nucleotides, or any combination thereof.

This invention further contemplates pharmaceutical compositions containing the nucleic acid molecules and a pharmaceutically acceptable carrier. The carrier may be a lipid molecule or liposome.

Embodiments of this invention further include a vector or cell containing the nucleic acid molecules.

This invention also contemplates methods for preventing, treating or ameliorating a disease in a subject in need by gene silencing, by administering to the subject a composition containing the nucleic acid molecules. The disease can be malignant tumor, cancer, sarcoma, or carcinoma.

Embodiments of this invention include use of a composition of the nucleic acid molecules for preventing, ameliorating or treating a disease or condition in a subject in need.

In certain embodiments, a composition of this invention may be for use in medical therapy, or for use in the treatment of the human or animal body.

In additional embodiments, a composition of this invention may be for preparing or manufacturing a medicament for preventing, ameliorating or treating a disease or condition in a subject in need.

### BRIEF DESCRIPTION OF THE DRAWINGS

FIG. 1: FIG. 1 shows the profound reduction of orthotopic lung cancer tumors in vivo by a siRNA of this invention targeted to GST-π. The GST-π siRNA was administered in a liposomal formulation at a dose of 2 mg/kg to athymic nude mice presenting A549 orthotopic lung cancer tumors. Final primary tumor weights were measured at necropsy for the treatment group and a vehicle control group. The GST-π siRNA showed significant efficacy for inhibition of lung cancer tumors in this six-week study. As shown in FIG. 1, after 43 days, the GST-π siRNA showed markedly advantageous tumor inhibition, with final primary tumor average weights significantly reduced by 2.8-fold, as compared to control.

FIG. 2: FIG. 2 shows tumor inhibition efficacy in vivo for a GST-π siRNA. A cancer xenograft model using A549 cells was utilized with a relatively low dose of siRNA at 0.75 mg/kg. The GST-π siRNA showed advantageous tumor inhibition within a few days. After 36 days, the GST-π siRNA showed markedly advantageous tumor inhibition, with final tumor average volumes significantly reduced by about 2-fold, as compared to control.

FIG. 3: FIG. 3 shows tumor inhibition efficacy in vivo for a GST-π siRNA at the endpoint of FIG. 2. The GST-π siRNA showed advantageous tumor inhibition with average tumor weights reduced by more than 2-fold.

FIG. 4: FIG. 4 shows that a GST-π siRNA of this invention greatly increased cancer cell death by apoptosis in vitro. The GST-π siRNA caused upregulation of PUMA, a biomarker for apoptosis, which is associated with loss in cell viability. In FIG. 4, the expression of PUMA was greatly increased from 2-6 days after transfection of the GST-π SiRNA.

FIG. 5: FIG. 5 shows that a GST-π siRNA of this invention provided knockdown efficacy for A549 xenograft tumors in vivo. Dose dependent knockdown of GST-π mRNA was observed in athymic nude (nu/nu) female mice (Charles River) with the siRNA targeted to GST-π. As shown in FIG. 5, at a dose of 4 mg/kg, significant reduction of about 40% in GST-π mRNA was detected 24 hours after injection.

FIG. 6: FIG. 6 shows that a GST-π siRNA of this invention inhibited pancreatic cancer xenograft tumors in vivo. The GST-π siRNA provided gene silencing potency in vivo when administered in a liposomal formulation to pancreatic cancer xenograft tumors in athymic nude female mice, 6 to 8 weeks old. As shown in FIG. 6, a dose response was obtained with doses ranging from 0.375 mg/kg to 3 mg/kg of siRNA targeted to GST-π. The GST-π siRNA showed advantageous tumor inhibition within a few days after administration, the tumor volume being reduced by about 2-fold at the endpoint.

FIG. 7: FIG. 7 shows that a GST-π siRNA of this invention exhibited increased serum stability. As shown in FIG. 7, the half-life (tv) in serum for both the sense strand (FIG. 7, top) and antisense strand (FIG. 7, bottom) of a GST-π siRNA was about 100 minutes.

FIG. 8: FIG. 8 shows that a GST-π siRNA of this invention exhibited enhanced stability in formulation in plasma. FIG. 8 shows incubation of a liposomal formulation of a GST-π siRNA in 50% human serum in PBS, and detection of remaining siRNA at various time points. As shown in FIG. 8, the half-life (t_{1/2}) in plasma of the formulation of the GST-π siRNA was significantly longer than 100 hours.

FIG. 9: FIG. 9 shows in vitro knockdown for the guide strand of a GST-π siRNA. As shown in FIG. 9, the guide strand knockdown of the GST-π siRNA was approximately exponential, as compared to a control with scrambled sequence that exhibited no effect.

FIG. 10: FIG. 10 shows in vitro knockdown for the passenger strand of the GST-π SiRNA of FIG. 9. As shown in FIG. 10, the passenger strand off target knockdown for the GST-π siRNA was greatly reduced, with essentially no effect.

FIG. 11: FIG. 11 shows in vitro knockdown for the guide strands of several highly active GST-π siRNAs. As shown in FIG. 11, the guide strand knockdown activities of the GST-π siRNAs were approximately exponential.

FIG. 12: FIG. 12 shows in vitro knockdown for the passenger strand of the GST-π siRNAs of FIG. 11. As shown in FIG. 12, the passenger strand off target knockdown activities for the GST-π siRNAs were significantly reduced below about 500 pM.

FIG. 13: FIG. 13 shows in vitro knockdown for the guide strand of a highly active GST-π SiRNA. As shown in FIG. 13, the guide strand knockdown activity of the GST-π siRNA was approximately exponential.

FIG. 14: FIG. 14 shows in vitro knockdown for the passenger strand of the GST-π SiRNA of FIG. 13. As shown in FIG. 14, the passenger strand off target knockdown activity for the GST-π siRNA was significantly reduced.

FIG. 15: FIG. 15 shows tumor inhibition efficacy in vivo for a p21 siRNA. A cancer xenograft model using A549 cells was utilized with a relatively low dose of siRNA at 0.75 mg/kg. The p21 siRNA showed advantageous tumor inhibition within a few days. After 30 days, the GST-π siRNA showed markedly advantageous tumor inhibition, with final tumor average volumes significantly reduced by more than 2-fold, as compared to control.

### DETAILED DESCRIPTION OF THE PREFERRED EMBODIMENT

This invention relates to compounds, compositions and methods for nucleic acid based therapeutics for modulating gene expression.

In some embodiments, this invention provides molecules active in RNA interference, as well as structures and compositions that can silence expression of a gene.

The structures and compositions of this disclosure can be used in preventing or treating various diseases.

In further embodiments, this invention provides compositions for delivery and uptake of one or more therapeutic RNAi molecules of this invention, as well as methods of use thereof. The RNA-based compositions of this invention can be used in methods for preventing or treating malignant tumors, such as cancers.

Therapeutic compositions of this invention include nucleic acid molecules that are active in RNA interference. The therapeutic nucleic acid molecules can be targeted to various genes for gene silencing.

In various embodiments, this invention provides a range of molecules that can be active as a small interfering RNA (siRNA), and can regulate or silence gene expression.

Embodiments of this invention further provide a vehicle, formulation, or lipid nanoparticle formulation for delivery of the inventive siRNAs to subjects in need of preventing or treating a disease. This invention further contemplates methods for administering siRNAs as therapeutics to mammals.

The therapeutic molecules and compositions of this invention can be used for RNA interference directed to preventing or treating a disease, by administering a compound or composition to a subject in need.

This invention provides a range of RNAi molecules, where each molecule has a polynucleotide sense strand and a polynucleotide antisense strand; each strand of the molecule is from 15 to 30 nucleotides in length; a contiguous region of from 15 to 30 nucleotides of the antisense strand is at least partly complementary to a sequence of an mRNA; and at least a portion of the sense strand is complementary to at least a portion of the antisense strand, and the molecule has a duplex region of from 15 to 30 nucleotides in length.

A RNAi molecule of this invention can have a contiguous region of from 15 to 30 nucleotides of the antisense strand that is complementary to a sequence of a human mRNA, which is located in the duplex region of the molecule.

In some embodiments, a RNAi molecule can have a contiguous region of from 15 to 30 nucleotides of the antisense strand that is complementary to a sequence of a human mRNA.

Embodiments of this invention may further provide methods for preventing, treating or ameliorating one or more symptoms of a disease or condition, or reducing the risk of developing a disease or condition, or delaying the onset of a disease or condition in a mammal in need thereof.

### RNAi Structures for Universal Genomic Silencing

Embodiments of this invention can provide RNAi molecules targeted to any gene, which exhibit high activity for silencing expression of the gene.

RNAi molecules of this disclosure can exhibit surprisingly high activity for silencing expression of a gene, while providing reduced off target effects.

In some embodiments, an RNAi molecule of this invention contains one or more 2′-deoxy nucleotides. In these embodiments, the one or more 2′-deoxy nucleotides can be located in the seed region of the siRNA.

The one or more 2′-deoxy nucleotides can be mono-deoxynucleotides.

As used herein, deoxynucleotide refers to a mono-2′-deoxy nucleotide.

A 2′-deoxy nucleotide may be substituted at the 2′ position with a halogen.

In certain embodiments, an RNAi molecule of this invention contains one or more 2′-deoxy nucleotides in the antisense or guide strand of the siRNA. More particularly, the one or more deoxynucleotides can be located at positions 1 to 8 from the 5′ end of the antisense strand of the siRNA. In certain embodiments, the one or more deoxynucleotides can be located at positions 2 to 8 from the 5′ end of the antisense strand of the siRNA. In additional embodiments, the one or more deoxynucleotides can be located at positions 3 to 8 from the 5′ end of the antisense strand of the siRNA.

This invention contemplates siRNA structures that can have an antisense strand containing deoxynucleotides in a plurality of positions.

In some embodiments, an siRNA structure can have an antisense strand containing deoxynucleotides at each of positions 4, 6 and 8 from the 5′ end of the antisense strand.

In further embodiments, an siRNA structure can have an antisense strand containing deoxynucleotides at each of positions 3, 5 and 7 from the 5′ end of the antisense strand.

In additional embodiments, an siRNA structure can have an antisense strand containing deoxynucleotides at each of positions 1, 3, 5 and 7 from the 5′ end of the antisense strand.

In certain embodiments, an siRNA structure can have an antisense strand containing deoxynucleotides at each of positions 3 to 8 from the 5′ end of the antisense strand.

In some embodiments, an siRNA structure can have an antisense strand containing deoxynucleotides at each of positions 5 to 8, from the 5′ end of the antisense strand.

Any of these structures can be combined with one or more modified or chemically modified nucleotides in other positions.

The RNAi molecules of this invention can inhibit expression of the mRNA of a gene with an advantageous IC50 of less than about 200 μM. In certain embodiments, the RNAi molecules of this invention can inhibit expression of the mRNA of a gene with an advantageous IC50 of less than about 100 μM, or less than about 50 μM, or less than about 30 μM, or less than about 20 μM, or less than about 10 μM, or less than about 5 pM, or less than about 1 μM.

In further embodiments, the RNAi molecules of this invention can inhibit expression of the mRNA level of a gene by at least 25% in vivo, upon a single administration.

An siRNA of this invention can be targeted to any gene.

Examples of genes to which an siRNA of this invention can be targeted include genes listed in the Gene Families Index of the HUGO Gene Nomenclature Committee.

Examples of genes to which an siRNA of this invention can be targeted include nuclear genes, mitochondrial genes, protein coding genes, proto-oncogenes, oncogenes, tumor suppressor genes, and cell signaling genes.

Examples of genes to which an siRNA of this invention can be targeted include genes expressing any member or sub-member of the human family of proteins including SRY, beta-globin, RAS, cytosolic GST, mitochondrial GST, MAPEG GST, GST-π, p16, p21, p53, serum albumin, Type VII collagen, Complement C3, Apolipoprotein B, phenylalanine hydroxylase, Factor VIII, Huntingtin, RB1 retinoblastoma protein, CFTR, Titin, Utrophin, and Dystrophin.

Modified and Chemically-Modified siRNAs

Embodiments of this invention encompass siRNA molecules that are modified or chemically-modified to provide enhanced properties for therapeutic use, such as increased activity and potency for gene silencing. This invention provides modified or chemically-modified siRNA molecules that can have increased serum stability, as well as reduced off target effects, without loss of activity and potency of the siRNA molecules for gene modulation and gene silencing. In some aspects, this invention provides siRNAs having modifications or chemical modifications in various combinations, which enhance the stability and efficacy of the siRNA.

As used herein, the terms modified and chemically-modified refer to changes made in the structure of a naturally-occurring nucleotide or nuclei acid structure of an siRNA, which encompasses siRNAs having one or more nucleotide analogs, altered nucleotides, non-standard nucleotides, non-naturally occurring nucleotides, and combinations thereof.

In some embodiments, the number of modified or chemically-modified structures in an siRNA can include all of the structural components, and/or all of the nucleotides of the siRNA molecule.

Examples of modified and chemically-modified siRNAs include siRNAs having modification of the sugar group of a nucleotide, modification of a nucleobase of a nucleotide, modification of a nucleic acid backbone or linkage, modification of the structure of a nucleotide or nucleotides at the terminus of a siRNA strand, and combinations thereof.

Examples of modified and chemically-modified siRNAs include siRNAs having modification of the substituent at the 2′ carbon of the sugar.

Examples of modified and chemically-modified siRNAs include siRNAs having modification at the 5′ end, the 3′ end, or at both ends of a strand.

Examples of modified and chemically-modified siRNAs include siRNAs having modifications that produce complementarity mismatches between the strands.

Examples of modified and chemically-modified siRNAs include siRNAs having a 5′-propylamine end, a 5′-phosphorylated end, a 3′-puromycin end, or a 3′-biotin end group.

Examples of modified and chemically-modified siRNAs include siRNAs having a 2′-fluoro substituted ribonucleotide, a 2′-OMe substituted ribonucleotide, a 2′-deoxy ribonucleotide, a 2′-amino substituted ribonucleotide, a 2′-thio substituted ribonucleotide.

Examples of modified and chemically-modified siRNAs include siRNAs having one or more 5-halouridines, 5-halocytidines, 5-methylcytidines, ribothymidines, 2-aminopurines, 2,6-diaminopurines, 4-thiouridines, or 5-aminoallyluridines.

Examples of modified and chemically-modified siRNAs include siRNAs having one or more phosphorothioate groups.

Examples of modified and chemically-modified siRNAs include siRNAs having one or more 2′-fluoro substituted ribonucleotides, 2′-fluorouridines, 2′-fluorocytidines, 2′-deoxyribonucleotides, 2′-deoxyadenosines, or 2′-deoxyguanosines.

Examples of modified and chemically-modified siRNAs include siRNAs having one or more phosphorothioate linkages.

Examples of modified and chemically-modified siRNAs include siRNAs having one or more alkylene diol linkages, oxy-alkylthio linkages, or oxycarbonyloxy linkages.

Examples of modified and chemically-modified siRNAs include siRNAs having one or more deoxyabasic groups, inosines, N3-methyl-uridines, N6,N6-dimethyl-adenosines, pseudouridines, purine ribonucleosides, and ribavirins.

Examples of modified and chemically-modified siRNAs include siRNAs having one or more 3′ or 5′ inverted terminal groups.

Examples of modified and chemically-modified siRNAs include siRNAs having one or more 5-(2-amino) propyluridines, 5-bromouridines, adenosines, 8-bromo guanosines, 7-deaza-adenosines, or N6-methyl adenosine.

### GST-π and RNAi Molecules

Various human cancer tissues have been found to correlate with the appearance of mutated KRAS gene. In some cases, the tissues also present an elevated level of Glutathione S-Transferase Pi (GST-π) expression. (Miyanishi et al., Gastroenterology, 2001, Vol. 121:865-874, Abstract) For example, elevated serum GST-π levels were observed in patients with various gastrointestinal malignancies. (Niitsu et al., Cancer, 1989, Vol. 63, No. 2, pp. 317-323, Abstract)

GST-π is a member of a GST family of enzymes that play a role in detoxification by catalyzing the conjugation of hydrophobic and electrophilic compounds with reduced glutathione. GST-π expression can be reduced in vitro with a siRNA. (Niitsu et al., US 2014/0315975 A1). However, there are many drawbacks of existing siRNA agents, such as insufficient activity, off-target effects, lack of serum stability, and lack of in vivo potency of efficacy.

The nucleic acid sequence of an example target human glutathione S-transferase pi (human GST-π) mRNA is disclosed in GenBank accession number NM_000852.3 (hGSTP1), and is 986 nucleotides in length.

One of ordinary skill in the art would understand that a reported sequence may change over time and to incorporate any changes needed in the nucleic acid molecules herein accordingly.

Embodiments of this invention can provide compositions and methods for gene silencing of GST-π expression using small nucleic acid molecules. Examples of nucleic acid molecules include molecules active in RNA interference (RNAi molecules), short interfering RNA (siRNA), double-stranded RNA (dsRNA), micro-RNA (miRNA), and short hairpin RNA (shRNA) molecules, as well as DNA-directed RNAs (ddRNA), Piwi-interacting RNAs (piRNA), and repeat associated siRNAs (rasiRNA). Such molecules are capable of mediating RNA interference against GST-π gene expression.

The composition and methods disclosed herein can also be used in treating various kinds of malignant tumors in a subject.

The nucleic acid molecules and methods of this invention may be used to down regulate the expression of genes that encode GST-π.

The compositions and methods of this invention can include one or more nucleic acid molecules, which, independently or in combination, can modulate or regulate the expression of GST-π protein and/or genes encoding GST-π proteins, proteins and/or genes encoding GST-π associated with the maintenance and/or development of diseases, conditions or disorders associated with GST-π, such as malignant tumor.

The compositions and methods of this invention are described with reference to exemplary sequences of GST-π. A person of ordinary skill in the art would understand that various aspects and embodiments of the invention are directed to any related GST-π genes, sequences, or variants, such as homolog genes and transcript variants, and polymorphisms, including single nucleotide polymorphism (SNP) associated with any GST-π genes.

In some embodiments, the compositions and methods of this invention can provide a double-stranded short interfering nucleic acid (siRNA) molecule that downregulates the expression of a GST-π gene, for example human GST-π.

A RNAi molecule of this invention can be targeted to GST-π and any homologous sequences, for example, using complementary sequences or by incorporating non-canonical base pairs, for example, mismatches and/or wobble base pairs, that can provide additional target sequences.

In instances where mismatches are identified, non-canonical base pairs, for example, mismatches and/or wobble bases can be used to generate nucleic acid molecules that target more than one gene sequence.

For example, non-canonical base pairs such as UU and CC base pairs can be used to generate nucleic acid molecules that are capable of targeting sequences for differing GST-π targets that share sequence homology. Thus, a RNAi molecule can be targeted to a nucleotide sequence that is conserved between homologous genes, and a single RNAi molecule can be used to inhibit expression of more than one gene.

In some aspects, the compositions and methods of this invention include RNAi molecules that are active against GST-π mRNA, where the RNAi molecule includes a sequence complementary to any mRNA encoding a GST-π sequence.

In some embodiments, a RNAi molecule of this disclosure can have activity against GST-π RNA, where the RNAi molecule includes a sequence complementary to an RNA having a variant GST-π encoding sequence, for example, a mutant GST-π gene known in the art to be associated with malignant tumor.

In further embodiments, a RNAi molecule of this invention can include a nucleotide sequence that can interact with a nucleotide sequence of a GST-π gene and mediate silencing of GST-π gene expression.

The nucleic acid molecules for inhibiting expression of GST-π may have a sense strand and an antisense strand, wherein the strands form a duplex region. The nucleic acid molecules may have one or more of the nucleotides in the duplex region being modified or chemically-modified, including such modifications as are known in the art. Any nucleotide in an overhang of the siRNA may also be modified or chemically-modified.

In some embodiments, the preferred modified or chemically-modified nucleotides are 2′-deoxy nucleotides. In additional embodiments, the modified or chemically-modified nucleotides can include 2′-O-alkyl substituted nucleotides, 2′-deoxy-2′-fluoro substituted nucleotides, phosphorothioate nucleotides, locked nucleotides, or any combination thereof.

In certain embodiments, a preferred structure can have an antisense strand containing deoxynucleotides in a plurality of positions, the plurality of positions being one of the following: each of positions 4, 6 and 8, from the 5′ end of the antisense strand; each of positions 3, 5 and 7, from the 5′ end of the antisense strand; each of positions 1, 3, 5 and 7, from the 5′ end of the antisense strand; each of positions 3-8, from the 5′ end of the antisense strand; and each of positions 5-8, from the 5′ end of the antisense strand. Any of these structures can be combined with one or more 2′-deoxy-2′-fluoro substituted nucleotides in the duplex region.

The nucleic acid molecules of this invention can inhibit expression of GST-π mRNA with an advantageous IC50 of less than about 200 μM. Further, the nucleic acid molecules can inhibit expression of GST-π mRNA levels by at least 25% in vivo, upon a single administration.

Pharmaceutical compositions are contemplated in this invention, which can contain one or more siRNAs as described herein, in combination with a pharmaceutically acceptable carrier. Any suitable carrier may be used, including those known in the art, as well as lipid molecules, nanoparticles, or liposomes, any of which may encapsulate the siRNA molecules.

This invention discloses methods for treating a disease associated with GST-π expression, which methods include administering to a subject in need a composition containing one or more of the siRNAs. Diseases to be treated may include malignant tumor, cancer, cancer caused by cells expressing mutated KRAS, sarcoma, and carcinoma, among others.

Examples of RNAi molecules of this invention targeted to GST-π mRNA are shown in Table 1.

TABLE 1
RNAi molecule sequences for GST-π

| | | SEQ | SENSE STRAND | SEQ | ANTISENSE STRAND |
|---|---|---|---|---|---|
| | Ref | ID | (5′-->3′) | ID | (5′-->3′) |
| ID | Pos | NO | SEQ ID NOS: 1 to 65 | NO | SEQ ID NOS: 66 to 130 |
| A1 | 652 | 1 | UCCCAGAACCAGGGAGGCAtt | 66 | UGCCUCCCUGGUUCUGGGAca |
| A10 | 635 | 2 | CUUUUGAGACCCUGCUGUCtt | 67 | GACAGCAGGGUCUCAAAAGgc |
| A11 | 649 | 3 | CUGUCCCAGAACCAGGGAGtt | 68 | CUCCCUGGUUCUGGGACAGca |
| A12 | 650 | 4 | UGUCCCAGAACCAGGGAGGtt | 69 | CCUCCCUGGUUCUGGGACAgc |
| A13 | 631 | 5 | AAGCCUUUUGAGACCCUGCtt | 70 | GCAGGGUCUCAAAAGGCUUca |
| A14 | 638 | 6 | UUGAGACCCUGCUGUCCCAtt | 71 | UGGGACAGCAGGGUCUCAAaa |
| A15 | 636 | 7 | UUUUGAGACCCUGCUGUCCtt | 72 | GGACAGCAGGGUCUCAAAAgg |
| A16 | 640 | 8 | GAGACCCUGCUGUCCCAGAtt | 73 | UCUGGGACAGCAGGGUCUCaa |
| A17 | 332 | 9 | GCUGGAAGGAGGAGGUGGUtt | 74 | ACCACCUCCUCCUUCCAGCtc |
| A18 | 333 | 10 | CUGGAAGGAGGAGGUGGUGtt | 75 | CACCACCUCCUCCUUCCAGct |
| A19 | 321 | 11 | UCAGGGCCAGAGCUGGAAGtt | 76 | CUUCCAGCUCUGGCCCUGAtc |
| A2 | 639 | 12 | UGAGACCCUGCUGUCCCAGtt | 77 | CUGGGACAGCAGGGUCUCAaa |
| A20 | 323 | 13 | AGGGCCAGAGCUGGAAGGAtt | 78 | UCCUUCCAGCUCUGGCCCUga |
| A21 | 331 | 14 | AGCUGGAAGGAGGAGGUGGtt | 79 | CCACCUCCUCCUUCCAGCUct |
| A22 | 641 | 15 | AGACCCUGCUGUCCCAGAAtt | 80 | UUCUGGGACAGCAGGGUCUca |
| A23 | 330 | 16 | GAGCUGGAAGGAGGAGGUGtt | 81 | CACCUCCUCCUUCCAGCUCtg |
| A25 | 647 | 17 | UGCUGUCCCAGAACCAGGGtt | 82 | CCCUGGUUCUGGGACAGCAgg |
| A26 | 653 | 18 | CCCAGAACCAGGGAGGCAAtt | 83 | UUGCCUCCCUGGUUCUGGGac |
| A3 | 654 | 19 | CCAGAACCAGGGAGGCAAGtt | 84 | CUUGCCUCCCUGGUUCUGGga |
| A4 | 637 | 20 | UUUGAGACCCUGCUGUCCCtt | 85 | GGGACAGCAGGGUCUCAAAag |
| A5 | 642 | 21 | GACCCUGCUGUCCCAGAACtt | 86 | GUUCUGGGACAGCAGGGUCtc |
| A6 | 319 | 22 | GAUCAGGGCCAGAGCUGGAtt | 87 | UCCAGCUCUGGCCCUGAUCtg |
| A7 | 632 | 23 | AGCCUUUUGAGACCCUGCUtt | 88 | AGCAGGGUCUCAAAAGGCUtc |
| A8 | 633 | 24 | GCCUUUUGAGACCCUGCUGtt | 89 | CAGCAGGGUCUCAAAAGGCtt |
| A9 | 634 | 25 | CCUUUUGAGACCCUGCUGUtt | 90 | ACAGCAGGGUCUCAAAAGGct |
| AG7 | 632 | 26 | CGCCUUUUGAGACCCUGCAtt | 91 | UGCAGGGUCUCAAAAGGCGtc |
| AK1 | 257 | 27 | CCUACACCGUGGUCUAUUUtt | 92 | AAAUAGACCACGGUGUAGGgc |
| AK10 | 681 | 28 | UGUGGGAGACCAGAUCUCCtt | 93 | GGAGAUCUGGUCUCCCACAat |
| AK11 | 901 | 29 | GCGGGAGGCAGAGUUUGCCtt | 94 | GGCAAACUCUGCCUCCCGCtc |
| AK12 | 922 | 30 | CCUUUCUCCAGGACCAAUAtt | 95 | UAUUGGUCCUGGAGAAAGGaa |
| AK13/ | 643 | 31 | ACCCUGCUGUCCCAGAACCtt | 96 | GGUUCUGGGACAGCAGGGUct |
| A24 | | | | | |
| AK2 | 267 | 32 | GGUCUAUUUCCCAGUUCGAtt | 97 | UCGAACUGGGAAAUAGACCac |
| AK3 | 512 | 33 | CCCUGGUGGACAUGGUGAAtt | 98 | UUCACCAUGUCCACCAGGGct |
| AK4 | 560 | 34 | ACAUCUCCCUCAUCUACACtt | 99 | GUGUAGAUGAGGGAGAUGUat |
| AK5 | 593 | 35 | GCAAGGAUGACUAUGUGAAtt | 100 | UUCACAUAGUCAUCCUUGCcc |
| AK6 | 698 | 36 | CCUUCGCUGACUACAACCUtt | 101 | AGGUUGUAGUCAGCGAAGGag |
| AK7 | 313 | 37 | CUGGCAGAUCAGGGCCAGAtt | 102 | UCUGGCCCUGAUCUGCCAGca |
| AK8 | 421 | 38 | GACGGAGACCUCACCCUGUtt | 103 | ACAGGGUGAGGUCUCCGUCct |
| AK9 | 590 | 39 | CGGGCAAGGAUGACUAUGUtt | 104 | ACAUAGUCAUCCUUGCCCGcc |
| AU10 | 635 | 40 | CUUUUGAGACCCUGCUGUAtt | 105 | UACAGCAGGGUCUCAAAAGgc |
| AU23 | 330 | 41 | GAGCUGGAAGGAGGAGGUAtt | 106 | UACCUCCUCCUUCCAGCUCtg |
| AU24 | 643 | 42 | ACCCUGCUGUCCCAGAACAtt | 107 | UGUUCUGGGACAGCAGGGUct |
| AU25 | 648 | 43 | UGCUGUCCCAGAACCAGGAtt | 108 | UCCUGGUUCUGGGACAGCAgg |
| AU7 | 632 | 44 | AGCCUUUUGAGACCCUGCAtt | 109 | UGCAGGGUCUCAAAAGGCUtc |
| AU9 | 634 | 45 | CCUUUUGAGACCCUGCUGAtt | 110 | UCAGCAGGGUCUCAAAAGGct |
| B1 | 629 | 46 | UGAAGCCUUUUGAGACCCUtt | 111 | AGGGUCUCAAAAGGCUUCAgt |
| B10 | 627 | 47 | ACUGAAGCCUUUUGAGACCtt | 112 | GGUCUCAAAAGGCUUCAGUtg |
| B11 | 596 | 48 | AGGAUGACUAUGUGAAGGCtt | 113 | GCCUUCACAUAGUCAUCCUtg |
| B12 | 597 | 49 | GGAUGACUAUGUGAAGGCAtt | 114 | UGCCUUCACAUAGUCAUCCtt |
| B13 | 598 | 50 | GAUGACUAUGUGAAGGCACtt | 115 | GUGCCUUCACAUAGUCAUCct |
| B14 | 564 | 51 | CUCCCUCAUCUACACCAACtt | 116 | GUUGGUGUAGAUGAGGGAGat |
| B2 | 630 | 52 | GAAGCCUUUUGAGACCCUGtt | 117 | CAGGGUCUCAAAAGGCUUCag |
| B3 | 563 | 53 | UCUCCCUCAUCUACACCAAtt | 118 | UUGGUGUAGAUGAGGGAGAtg |
| B4 | 567 | 54 | CCUCAUCUACACCAACUAUtt | 119 | AUAGUUGGUGUAGAUGAGGga |
| B5 | 566 | 55 | CCCUCAUCUACACCAACUAtt | 120 | UAGUUGGUGUAGAUGAGGGag |
| B6 | 625 | 56 | CAACUGAAGCCUUUUGAGAtt | 121 | UCUCAAAAGGCUUCAGUUGCC |
| B7 | 626 | 57 | AACUGAAGCCUUUUGAGACtt | 122 | GUCUCAAAAGGCUUCAGUUgc |
| B8 | 628 | 58 | CUGAAGCCUUUUGAGACCCtt | 123 | GGGUCUCAAAAGGCUUCAGtt |
| B9 | 565 | 59 | UCCCUCAUCUACACCAACUtt | 124 | AGUUGGUGUAGAUGAGGGAga |
| BG3 | 563 | 60 | GCUCCCUCAUCUACACCAAtt | 125 | UUGGUGUAGAUGAGGGAGCtg |
| BU2 | 630 | 61 | GAAGCCUUUUGAGACCCUAtt | 126 | UAGGGUCUCAAAAGGCUUCag |
| BU10 | 627 | 62 | ACUGAAGCCUUUUGAGACAtt | 127 | UGUCUCAAAAGGCUUCAGUtg |
| BU14 | 565 | 63 | CUCCCUCAUCUACACCAAAtt | 128 | UUUGGUGUAGAUGAGGGAGat |
| BU4 | 567 | 64 | CCUCAUCUACACCAACUAAtt | 129 | UUAGUUGGUGUAGAUGAGGga |
| C1- | 934 | 65 | ACCAAUAAAAUUUCUAAGAtt | 130 | UCUUAGAAAUUUUAUUGGUcc |
| 934 | | | | | |

Key for Table 1: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine respectively.

Examples of RNAi molecules of this invention targeted to GST-π mRNA are shown in Table 2.

TABLE 2
RNAi molecule sequences for GST-π

| | SEQ | SENSE STRAND | SEQ | ANTISENSE STRAND |
|---|---|---|---|---|
| | ID | (5′-->3′) | ID | (5′-->3′) |
| ID | NO | SEQ ID NOS: 131 to 156 | NO | SEQ ID NOS: 157 to 182 |
| BU2′ | 131 | GAAGCCUUUUGAGACCCUANN | 157 | UAGGGUCUCAAAAGGCUUCNN |
| 14 | 132 | GAAGCCUUUUGAGACCCUAUU | 158 | UAGGGUCUCAAAAGGCUUCUU |
| 15 | 133 | GAAGCCUUUUGAGACCCUAUU | 159 | uagggucuCAAAAGGCUUCUU |
| 16 | 134 | GAAGCCUUUUGAGACCCUAUU | 160 | UagggucuCAAAAGGCUUCUU |
| 17 | 135 | GAAGCCUUUUGAGACCCUAUU | 161 | UAgggucuCAAAAGGCUUCUU |
| 18 | 136 | GAAGCCUUUUGAGACCCUAUU | 162 | UAGggucuCAAAAGGCUUCUU |
| 19 | 137 | GAAGCCUUUUGAGACCCUAUU | 163 | UAGGgucuCAAAAGGCUUCUU |
| 20 | 138 | GAAGCCUUUUGAGACCCUAUU | 164 | uAgGgUcUCAAAAGGCUUCUU |
| 21 | 139 | GAAGCCUUUUGAGACCCUAUU | 165 | UAgGgUcUCAAAAGGCUUCUU |
| 22 | 140 | GAAGCCUUUUGAGACCCUAUU | 166 | UaGgGuCuCAAAAGGCUUCUU |
| 23 | 141 | GAAGCCUUUUGAGACCCUAUU | 167 | UAGgGuCuCAAAAGGCUUCUU |
| 24 | 142 | GAAGCCUUUUGAGACCCUAtt | 168 | UagggucuCAAAAGGCUUCUU |
| 25 | 143 | GAAGCCUUUUGAGACCCUAUU | 169 | UAGGGUCUCAAAAGGCUUCUU |
| 26 | 144 | GAAGCCUUUUGAGACCCUAUU | 170 | fUAGGGUCUCAAAAGGCUUCUU |
| 27 | 145 | GAAGCCUUUUGAGACCCUAUU | 171 | uAGGGUCUCAAAAGGCUUCUU |
| 28 | 146 | GAAGCCUUUUGAGACCCUAUU | 172 | UsAGGGUCUCAAAAGGCUUCUU |
| 29 | 147 | GAAGCCUUUUGAGACCCUfAUU | 173 | fUAGGGUCUFCAAAAGGCfUUCUU |
| 30 | 148 | GAAGCCUUUUGAGfACCCUfAUU | 174 | fUAGGGUCUfCAfAfAAGGCfUUCUU |
| 31 | 149 | GAAGCCUUUUGAGACCCUAUU | 175 | UAGGGUCUCAAAAGGCUUCUU |
| 31′ | 150 | GAAGCCUUUUGAGACCCUAUU | 176 | fUAGGGUCUCAAAAGGCUUCUU |
| 32 | 151 | GAAGCCUUUUGAGACCCUAUU | 177 | UAGGGUCUCAAAAGGCUUCUU |
| 39 | 152 | GAAGCCUUUUGAGACCCUAUU | 178 | UAGgGuCuCAAAAGGCUUCUU |
| 45 | 153 | GAAGCCUUUUGAGACCCUAUU | 179 | UAGgGuCuCAAAAGGCUUCUU |
| 46 | 154 | GAAGCCUUUUGAGACCCUAUU | 180 | UAGgGuCuCAAAAGGCUUCUU |
| 47 | 155 | GAAGCCUUUUGAGACCCUAUU | 181 | UAGgGuCuCAAAAGGCUUCUU |
| 48 | 156 | GAAGCCUUUUGAGACCCUAUU | 182 | fUAGgGuCuCAAAAGGCUUCUU |

Key for Table 2: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively.

Underlining refers to 2′-OMe-substituted, e.g., U. The lower case letter f refers to 2′-deoxy-2′-fluoro substitution, e.g. fU is 2′-deoxy-2′-fluoro-U. Nis A, C, G, U, U, a, c, g, u, t, or a modified, inverted, or chemically modified nucleotide.

Examples of RNAi molecules of this invention targeted to GST-π mRNA are shown in Table 3.

TABLE 3
RNAi molecule sequences for GST-π

| | SEQ | SENSE STRAND | SEQ | ANTISENSE STRAND |
|---|---|---|---|---|
| | ID | (5′-->3′) | ID | (5′-->3′) |
| ID | NO | SEQ ID NOS: 183 to 194 | NO | SEQ ID NOS: 195 to 206 |
| A9′ | 183 | CCUUUUGAGACCCUGCUGUNN | 195 | ACAGCAGGGUCUCAAAAGGNN |
| 1 | 184 | CCUCAUCUACACCAACUAUUU | 196 | AUAGUUGGUGUAGAUGAGGUU |
| 2 | 185 | CCUCAUCUACACCAACUAUUU | 197 | auaguuggUGUAGAUGAGGUU |
| 3 | 186 | CCUCAUCUACACCAACUAUUU | 198 | AuaguuggUGUAGAUGAGGUU |
| 4 | 187 | CCUCAUCUACACCAACUAUUU | 199 | AUaguuggUGUAGAUGAGGUU |
| 5 | 188 | CCUCAUCUACACCAACUAUUU | 200 | AUAguuggUGUAGAUGAGGUU |
| 6 | 189 | CCUCAUCUACACCAACUAUUU | 201 | AUAGuuggUGUAGAUGAGGUU |
| 7 | 190 | CCUCAUCUACACCAACUAUUU | 202 | aUaGuUgGUGUAGAUGAGGUU |
| 8 | 191 | CCUCAUCUACACCAACUAUUU | 203 | AUaGuUgGUGUAGAUGAGGUU |
| 9 | 192 | CCUCAUCUACACCAACUAUUU | 204 | AuAgUuGgUGUAGAUGAGGUU |
| 10 | 193 | CCUCAUCUACACCAACUAUUU | 205 | AUAgUuGgUGUAGAUGAGGUU |
| 11 | 194 | CCUCAUCUACACCAACUAUUU | 206 | AuaguuggUGUAGAUGAGGUU |

Key for Table 3: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively. Underlining refers to 2′-OMe-substituted, e.g., U. The lower case letter f refers to 2′-deoxy-2′-fluoro substitution, e.g. fU is 2′-deoxy-2′-fluoro-U. N is A, C, G, U, U, a, c, g, u, t, or a modified, inverted, or chemically modified nucleotide.

Examples of RNAi molecules of this invention targeted to GST-π mRNA are shown in Table 4.

TABLE 4
RNAi molecule sequences for GST-π

| | SEQ | SENSE STRAND | SEQ | ANTISENSE STRAND |
|---|---|---|---|---|
| | ID | (5′-->3′) | ID | (5′-->3′) |
| ID | NO | SEQ ID NOS: 207 to 221 | NO | SEQ ID NOS: 222 to 236 |
| B13′ | 207 | GAUGACUAUGUGAAGGCACNN | 222 | GUGCCUUCACAUAGUCAUCNN |
| 4 | 208 | GGAUGACUAUGUGAAGGCAUU | 223 | UGCCUUCACAUAGUCAUCCUU |
| 5 | 209 | GGAUGACUAUGUGAAGGCAUU | 224 | ugccuucaCAUAGUCAUCCUU |
| 6 | 210 | GGAUGACUAUGUGAAGGCAUU | 225 | UgccuucaCAUAGUCAUCCUU |
| 7 | 211 | GGAUGACUAUGUGAAGGCAUU | 226 | UGccuucaCAUAGUCAUCCUU |
| 8 | 212 | GGAUGACUAUGUGAAGGCAUU | 227 | UGCcuucaCAUAGUCAUCCUU |
| 9 | 213 | GGAUGACUAUGUGAAGGCAUU | 228 | UGCCuucaCAUAGUCAUCCUU |
| 10 | 214 | GGAUGACUAUGUGAAGGCAUU | 229 | uGcCuUcACAUAGUCAUCCUU |
| 11 | 215 | GGAUGACUAUGUGAAGGCAUU | 230 | UGcCuUcACAUAGUCAUCCUU |
| 12 | 216 | GGAUGACUAUGUGAAGGCAUU | 231 | UgCcUuCaCAUAGUCAUCCUU |
| 13 | 217 | GGAUGACUAUGUGAAGGCAUU | 232 | UGCcUuCaCAUAGUCAUCCUU |
| 14 | 218 | GGAUGACUAUGUGAAGGCAUU | 233 | UgccuucaCAUAGUCAUCCUU |
| 15 | 219 | GGAUGACUAUfGUfGAAGGCAUU | 234 | UGCICUUCACAUAGUCAUCCUU |
| 17 | 220 | GGAUGACUAUGUGAAGGCAUU | 235 | UGCCUUCACAUAGUCAUCCUU |
| 18 | 221 | GGAUGACUAUGUGAAGGCAUU | 236 | UGCCUUCACAUAGUCAUCCUU |

Key for Table 4: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively. Underlining refers to 2′-OMe-substituted, e.g., U. The lower case letter f refers to 2′-deoxy-2′-fluoro substitution, e.g. fU is 2′-deoxy-2′-fluoro-U. Nis A, C, G, U, U, a, c, g, u, t, or a modified, inverted, or chemically modified nucleotide.

Examples of RNAi molecules of this invention targeted to GST-π mRNA are shown in Table 5.

TABLE 5
RNAi molecule sequences for GST-π

| | SEQ | SENSE STRAND | SEQ | ANTISENSE STRAND |
|---|---|---|---|---|
| | ID | (5′-->3′) | ID | (5′-->3′) |
| ID | NO | SEQ ID NOS: 237 to 248 | NO | SEQ ID NOS: 249 to 260 |
| B2′ | 237 | GAAGCCUUUUGAGACCCUGNN | 249 | CAGGGUCUCAAAAGGCUUCNN |
| 1 | 238 | GAAGCCUUUUGAGACCCUGUU | 250 | CAGGGUCUCAAAAGGCUUCUU |
| 2 | 239 | GAAGCCUUUUGAGACCCUGUU | 251 | cagggucuCAAAAGGCUUCUU |
| 3 | 240 | GAAGCCUUUUGAGACCCUGUU | 252 | CagggucuCAAAAGGCUUCUU |
| 4 | 241 | GAAGCCUUUUGAGACCCUGUU | 253 | CAgggucuCAAAAGGCUUCUU |
| 5 | 242 | GAAGCCUUUUGAGACCCUGUU | 254 | CAGggucuCAAAAGGCUUCUU |
| 6 | 243 | GAAGCCUUUUGAGACCCUGUU | 255 | CAGGgucuCAAAAGGCUUCUU |
| 7 | 244 | GAAGCCUUUUGAGACCCUGUU | 256 | cAgGgUcUCAAAAGGCUUCUU |
| 8 | 245 | GAAGCCUUUUGAGACCCUGUU | 257 | CAgGgUcUCAAAAGGCUUCUU |
| 9 | 246 | GAAGCCUUUUGAGACCCUGUU | 258 | CaGgGuCuCAAAAGGCUUCUU |
| 10 | 247 | GAAGCCUUUUGAGACCCUGUU | 259 | CAGgGuCuCAAAAGGCUUCUU |
| 11 | 248 | GAAGCCUUUUGAGACCCUGUU | 260 | CagggucuCAAAAGGCUUCUU |

Key for Table 5: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively. Underlining refers to 2′-OMe-substituted, e.g., U. The lower case letter f refers to 2′-deoxy-2′-fluoro substitution, e.g. fU is 2′-deoxy-2′-fluoro-U. N is A, C, G, U, U, a, c, g, u, t, or a modified, inverted, or chemically modified nucleotide.

Examples of RNAi molecules of this invention targeted to GST-π mRNA are shown in Table 6.

TABLE 6
RNAi molecule sequences for GST-π

| | SEQ | SENSE STRAND | SEQ | ANTISENSE STRAND |
|---|---|---|---|---|
| | ID | (5′-->3′) | ID | (5′-->3′) |
| ID | NO | SEQ ID NOS: 261 to 272 | NO | SEQ ID NOS: 273 to 284 |
| B4′ | 261 | CCUCAUCUACACCAACUAUNN | 273 | AUAGUUGGUGUAGAUGAGGNN |
| 1 | 262 | CCUCAUCUACACCAACUAUUU | 274 | AUAGUUGGUGUAGAUGAGGUU |
| 2 | 263 | CCUCAUCUACACCAACUAUUU | 275 | auaguuggUGUAGAUGAGGUU |
| 3 | 264 | CCUCAUCUACACCAACUAUUU | 276 | AuaguuggUGUAGAUGAGGUU |
| 4 | 265 | CCUCAUCUACACCAACUAUUU | 277 | AUaguuggUGUAGAUGAGGUU |
| 5 | 266 | CCUCAUCUACACCAACUAUUU | 278 | AUAguuggUGUAGAUGAGGUU |
| 6 | 267 | CCUCAUCUACACCAACUAUUU | 279 | AUAGuuggUGUAGAUGAGGUU |
| 7 | 268 | CCUCAUCUACACCAACUAUUU | 280 | aUaGuUgGUGUAGAUGAGGUU |
| 8 | 269 | CCUCAUCUACACCAACUAUUU | 281 | AUaGuUgGUGUAGAUGAGGUU |
| 9 | 270 | CCUCAUCUACACCAACUAUUU | 282 | AuAgUuGgUGUAGAUGAGGUU |
| 10 | 271 | CCUCAUCUACACCAACUAUUU | 283 | AUAgUuGgUGUAGAUGAGGUU |
| 11 | 272 | CCUCAUCUACACCAACUAUUU | 284 | AuaguuggUGUAGAUGAGGUU |

Key for Table 6: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively. Underlining refers to 2′-OMe-substituted, e.g., U. The lower case letter f refers to 2′-deoxy-2′-fluoro substitution, e.g. fU is 2′-deoxy-2′-fluoro-U. Nis A, C, G, U, U, a, c, g, u, t, or a modified, inverted, or chemically modified nucleotide.

In some embodiments, this invention provides a range of nucleic acid molecules, wherein: a) the molecule has a polynucleotide sense strand and a polynucleotide antisense strand; b) each strand of the molecule is from 15 to 30 nucleotides in length; c) a contiguous region of from 15 to 30 nucleotides of the antisense strand is complementary to a sequence of an mRNA encoding GST-π; d) at least a portion of the sense strand is complementary to at least a portion of the antisense strand, and the molecule has a duplex region of from 15 to 30 nucleotides in length.

In some embodiments, the nucleic acid molecule can have contiguous region of from 15 to 30 nucleotides of the antisense strand that is complementary to a sequence of an mRNA encoding GST-π is located in the duplex region of the molecule.

In additional embodiments, the nucleic acid molecule can have a contiguous region of from 15 to 30 nucleotides of the antisense strand that is complementary to a sequence of an mRNA encoding GST-π.

In certain embodiments, each strand of the nucleic acid molecule can be from 18 to 22 nucleotides in length. The duplex region of the nucleic acid molecule can be 19 nucleotides in length.

In alternative forms, the nucleic acid molecule can have a polynucleotide sense strand and a polynucleotide antisense strand that are connected as a single strand, and form a duplex region connected at one end by a loop.

Some embodiments of a nucleic acid molecule of this disclosure can have a blunt end. In certain embodiments, a nucleic acid molecule can have one or more 3′ overhangs.

This invention provides a range of nucleic acid molecules that are RNAi molecules active for gene silencing. The inventive nucleic acid molecules can be a dsRNA, a siRNA, a micro-RNA, or a shRNA active for gene silencing, as well as a DNA-directed RNA (ddRNA), Piwi-interacting RNA (piRNA), or a repeat associated siRNA (rasiRNA). The nucleic acid molecules can be active for inhibiting expression of GST-π.

Embodiments of this invention further provide nucleic acid molecules having an IC50 for knockdown of GST-π of less than 100 μM.

Additional embodiments of this invention provide nucleic acid molecules having an IC50 for knockdown of GST-π of less than 50 μM.

This invention further contemplates compositions containing one or more of the inventive nucleic acid molecules, along with a pharmaceutically acceptable carrier. In certain embodiments, the carrier can be a lipid molecule or liposome.

The compounds and compositions of this invention are useful in methods for preventing or treating a GST-π associated disease, by administering a compound or composition to a subject in need.

The methods of this invention can utilize the inventive compounds for preventing or treating malignant tumor. The malignant tumor can be presented in various diseases, for example, cancers associated with GST-π expression, cancers caused by cells expressing mutated KRAS, sarcomas, fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, osteosarcoma, carcinomas, brain tumor, head and neck cancer, breast cancer, lung cancer, esophageal cancer, stomach cancer, duodenal cancer, appendix cancer, colorectal cancer, rectal cancer, liver cancer, pancreatic cancer, gallbladder cancer, bile duct cancer, anus cancer, kidney cancer, urethral cancer, urinary bladder cancer, prostate cancer, testicular cancer, uterine cancer, ovary cancer, skin cancer, leukemia, malignant lymphoma, epithelial malignant tumors, and non-epithelial malignant tumors.

### P21 and RNAi Molecules

p21 is a cell cycle-regulating protein that is encoded by CDKN1A gene and belongs to the CIP/KIP family. This protein has the function of inhibiting cell cycle progression at the G1 phase and the G2/M phase by inhibiting the effect of a cyclin-CDK complex through binding to the complex. Specifically, the p21 gene undergoes activation by p53, one of tumor suppressor genes. It has been reported that upon activation of p53 due to DNA damage or the like, p53 activates p21 so that the cell cycle is arrested at the G1 phase and the G2/M phase.

p21 is overexpressed in a variety of human cancers including prostate, cervical, breast and squamous cell carcinomas and, in many cases, p21 upregulation correlates positively with tumor grade, invasiveness and aggressiveness. See, e.g., Chang et al., Proc. Natl. Acad. Sci. USA, 2000, Vol. 97, No. 8, pp. 4291-96. Also, up-regulation of p21 has been reported to be associated with tumorigenicity and poor prognosis in many forms of cancers, including brain, prostate, ovarian, breast, and esophageal cell cancers. See, e.g., Winters et al., Breast Cancer Research, 2003, Vol. 5, No. 6, pp. R242-R249. Also, the disease can be age related diseases, including atherosclerosis, Alzheimer's disease, amyloidosis, and arthritis. See e.g., Chang et al., Proc. Natl. Acad. Sci. USA, 2000, Vol. 97, No. 8, pp. 4291-96.

p21 is present in various animals including humans. Sequence information for human CDKN1A (p21) is found at: NM_000389.4, NM_078467.2, NM_001291549.1, NM_001220778.1, NM_001220777.1 (NP_001207707.1, NP_001278478.1, NP_001207706.1, NP_510867.1, NP_000380.1).

The target human p21 mRNA is disclosed in GenBank accession number NM_000389.4 (CDKN1A), and is 2175 base pairs in length.

One of ordinary skill in the art would understand that a reported sequence may change over time and to incorporate any changes needed in the nucleic acid molecules herein accordingly.

Embodiments of this invention can provide compositions and methods for gene silencing of p21 expression using small nucleic acid molecules. Examples of nucleic acid molecules include molecules active in RNA interference (RNAi molecules), short interfering RNA (siRNA), micro-RNA (miRNA), and short hairpin RNA (shRNA) molecules, as well as DNA-directed RNA (ddRNA), Piwi-interacting RNA (piRNA), and repeat associated siRNA (rasiRNA). Such molecules are capable of mediating RNA interference against p21 gene expression.

The composition and methods disclosed herein can also be used in treating various kinds of malignant tumors in a subject.

The nucleic acid molecules and methods of this invention may be used to down regulate the expression of genes that encode p21.

The compositions and methods of this invention can include one or more nucleic acid molecules, which, independently or in combination, can modulate or regulate the expression of p21 protein and/or genes encoding p21 proteins, proteins and/or genes encoding p21 associated with the maintenance and/or development of diseases, conditions or disorders associated with p21, such as malignant tumor.

The compositions and methods of this invention are described with reference to exemplary sequences of p21. A person of ordinary skill in the art would understand that various aspects and embodiments of the invention are directed to any related p21 genes, sequences, or variants, such as homolog genes and transcript variants, and polymorphisms, including single nucleotide polymorphism (SNP) associated with any p21 genes.

In some embodiments, the compositions and methods of this invention can provide a double-stranded short interfering nucleic acid (siRNA) molecule that downregulates the expression of a p21 gene, for example human CDKN1A.

A RNAi molecule of this invention can be targeted to p21 and any homologous sequences, for example, using complementary sequences or by incorporating non-canonical base pairs, for example, mismatches and/or wobble base pairs, that can provide additional target sequences.

In instances where mismatches are identified, non-canonical base pairs, for example, mismatches and/or wobble bases can be used to generate nucleic acid molecules that target more than one gene sequence.

For example, non-canonical base pairs such as UU and CC base pairs can be used to generate nucleic acid molecules that are capable of targeting sequences for differing p21 targets that share sequence homology. Thus, a RNAi molecule can be targeted to a nucleotide sequence that is conserved between homologous genes, and a single RNAi molecule can be used to inhibit expression of more than one gene.

In some aspects, the compositions and methods of this invention include RNAi molecules that are active against p21 mRNA, where the RNAi molecule includes a sequence complementary to any mRNA encoding a p21 sequence.

In some embodiments, a RNAi molecule of this disclosure can have activity against p21 RNA, where the RNAi molecule includes a sequence complementary to an RNA having a variant p21 encoding sequence, for example, a mutant p21 gene known in the art to be associated with malignant tumor.

In further embodiments, a RNAi molecule of this invention can include a nucleotide sequence that can interact with a nucleotide sequence of a p21 gene and mediate silencing of p21 gene expression.

The nucleic acid molecules for inhibiting expression of p21 may have a sense strand and an antisense strand, wherein the strands form a duplex region. The nucleic acid molecules may have one or more of the nucleotides in the duplex region being modified or chemically-modified, including such modifications as are known in the art. Any nucleotide in an overhang of the siRNA may also be modified or chemically-modified.

In some embodiments, the preferred modified or chemically-modified nucleotides are 2′-deoxy nucleotides. In additional embodiments, the modified or chemically-modified nucleotides can include 2′-O-alkyl substituted nucleotides, 2′-deoxy-2′-fluoro substituted nucleotides, phosphorothioate nucleotides, locked nucleotides, or any combination thereof.

In certain embodiments, a preferred structure can have an antisense strand containing deoxynucleotides in a plurality of positions, the plurality of positions being one of the following: each of positions 4, 6 and 8, from the 5′ end of the antisense strand; each of positions 3, 5 and 7, from the 5′ end of the antisense strand; each of positions 1, 3, 5 and 7, from the 5′ end of the antisense strand; each of positions 3-8, from the 5′ end of the antisense strand; and each of positions 5-8, from the 5′ end of the antisense strand. Any of these structures can be combined with one or more 2′-deoxy-2′-fluoro substituted nucleotides in the duplex region.

The nucleic acid molecules of this invention can inhibit expression of p21 mRNA with an advantageous IC50 of less than about 200 μM. Further, the nucleic acid molecules can inhibit expression of p21 mRNA levels by at least 25% in vivo, upon a single administration.

Pharmaceutical compositions are contemplated in this invention, which can contain one or more siRNAs as described herein, in combination with a pharmaceutically acceptable carrier. Any suitable carrier may be used, including those known in the art, as well as lipid molecules, nanoparticles, or liposomes, any of which may encapsulate the siRNA molecules.

This invention discloses methods for treating a disease that may be associated with p21 expression, which methods include administering to a subject in need a composition containing one or more of the siRNAs. Diseases to be treated may include malignant tumor, cancer, cancer caused by cells expressing mutated KRAS, sarcoma, and carcinoma, among others.

Examples of RNAi molecules of this invention targeted to p21 mRNA are shown in Table 7.

TABLE 7
RNAi molecule sequences for p21

| | SEQ | SENSE STRAND | SEQ | ANTISENSE STRAND |
|---|---|---|---|---|
| Ref | ID | (5′-->3′) | ID | (5′-->3′) |
| Pos | NO | SEQ ID NOS: 285 to 312 | NO | SEQ ID NOS: 313 to 340 |
| 2085 | 285 | CUUAGUGACUUUACUUGUAmUmU | 313 | UACAAGUAAAGUCACUAAGmUmU |
| 500 | 286 | CAGACCAGCAUGACAGAUUmUmU | 314 | AAUCUGUCAUGCUGGUCUGmUmU |
| 540 | 287 | UGAUCUUCUCCAAGAGGAAmUmU | 315 | UUCCUCUUGGAGAAGAUCAmUmU |
| 1706 | 288 | GUUCAUUGCACUUUGAUUAmUmU | 316 | UAAUCAAAGUGCAAUGAACmUmU |
| 1709 | 289 | CAUUGCACUUUGAUUAGCAmUmU | 317 | UGCUAAUCAAAGUGCAAUGmUmU |
| 210 | 290 | AGCGAUGGAACUUCGACUUmUmU | 318 | AAGUCGAAGUUCCAUCGCUmUmU |
| 211 | 291 | GCGAUGGAACUUCGACUUUmUmU | 319 | AAAGUCGAAGUUCCAUCGCmUmU |
| 1473 | 292 | GGGAAGGGACACACAAGAAmUmU | 320 | UUCUUGUGUGUCCCUUCCCmUmU |
| 1507 | 293 | UCUACCUCAGGCAGCUCAAmUmU | 321 | UUGAGCUGCCUGAGGUAGAmUmU |
| 2067 | 294 | GGUGCUCAAUAAAUGAUUCmUmU | 322 | GAAUCAUUUAUUGAGCACCmUmU |
| 1063 | 295 | CAUCAUCAAAAACUUUGGAmUmU | 323 | UCCAAAGUUUUUGAUGAUGmUmU |
| 1735 | 296 | AAGGAGUCAGACAUUUUAAmUmU | 324 | UUAAAAUGUCUGACUCCUUmUmU |
| 783 | 297 | GUGCUGGGCAUUUUUAUUUmUmU | 325 | AAAUAAAAAUGCCCAGCACmUmU |
| 869 | 298 | GCCGGCUUCAUGCCAGCUAmUmU | 326 | UAGCUGGCAUGAAGCCGGCmUmU |
| 1060 | 299 | GGGCAUCAUCAAAAACUUUmUmU | 327 | AAAGUUUUUGAUGAUGCCCmUmU |
| 1492 | 300 | GAAGGGCACCCUAGUUCUAmUmU | 328 | UAGAACUAGGGUGCCCUUCmUmU |
| 1704 | 301 | CAGUUCAUUGCACUUUGAUmUmU | 329 | AUCAAAGUGCAAUGAACUGmUmU |
| 1733 | 302 | ACAAGGAGUCAGACAUUUUmUmU | 330 | AAAAUGUCUGACUCCUUGUmUmU |
| 1847 | 303 | UGGAGGCACUGAAGUGCUUmUmU | 331 | AAGCACUUCAGUGCCUCCAmUmU |
| 2000 | 304 | GCAGGGACCACACCCUGUAmUmU | 332 | UACAGGGUGUGGUCCCUGCmUmU |
| 2014 | 305 | CUGUACUGUUCUGUGUCUUmUmU | 333 | AAGACACAGAACAGUACAGmUmU |
| 677 | 306 | UUAAACACCUCCUCAUGUAmUmU | 334 | UACAUGAGGAGGUGUUUAAmUmU |
| 475 | 307 | AGACUCUCAGGGUCGAAAAmUmU | 335 | UUUUCGACCCUGAGAGUCUmUmU |
| 508 | 308 | CAUGACAGAUUUCUACCACmUmU | 336 | GUGGUAGAAAUCUGUCAUGmUmU |
| 514 | 309 | AGAUUUCUACCACUCCAAAmUmU | 337 | UUUGGAGUGGUAGAAAUCUmUmU |
| 549 | 310 | CCAAGAGGAAGCCCUAAUCmUmU | 338 | GAUUAGGGCUUCCUCUUGGmUmU |
| 382 | 311 | GACAGCAGAGGAAGACCAUmUmU | 339 | AUGGUCUUCCUCUGCUGUCmUmU |
| 2042 | 312 | CUCCCACAAUGCUGAAUAUmUmU | 340 | AUAUUCAGCAUUGUGGGAGmUmU |

Key for Table 7: Upper case A, G, C and U referred to for ribo-A, ribo-G, ribo-C and ribo-U respectively. The lower case letters a, g, c, t represent 2′-deoxy-A, 2′-deoxy-G, 2′-deoxy-C and thymidine respectively. mU is 2′-methoxy-U.

Examples of RNAi molecules of this invention targeted to p21 mRNA are shown in Table 8.

TABLE 8
RNAi molecule sequences for p21

| | SEQ | SENSE STRAND | SEQ | ANTISENSE STRAND |
|---|---|---|---|---|
| Ref | ID | (5′-->3′) | ID | (5′-->3′) |
| Pos | NO | SEQ ID NOS: 341 to 354 | NO | SEQ ID NOS: 355 to 368 |
| 1735′ | 341 | AAGGAGUCAGACAUUUUAANN | 355 | UUAAAAUGUCUGACUCCUUNN |
| 1 | 342 | AAGGAGUCAGACAUUUUAAUU | 356 | UUAaAaUgUCUGACUCCUUUU |
| 2 | 343 | AAGGAGUCAGACAUUUUAAUU | 357 | UUAaAaUgUCUGACUCCUUUU |
| 3 | 344 | AAGGAGUCAGACAUUUUAAUU | 358 | UUAaAaUgUCUGACUCCUUUU |
| 4 | 345 | AAGGAGUCAGACAUUUUAAUU | 359 | UUAaAaUgUCUGACUCCUUUU |
| 5 | 346 | AAGGAGUCAGACAUUUUAAUU | 360 | UUaaaaugUCUGACUCCUUUU |
| 6 | 347 | AAGGAGUCAGACAUUUUAAUU | 361 | UUAAaaugUCUGACUCCUUUU |
| 7 | 348 | AAGGAGUCAGACAUUUUAAUU | 362 | uUaAaAuGUCUGACUCCUUUU |
| 8 | 349 | AAGGAGUCAGACAUUUUAAUU | 363 | UUaAaAuGUCUGACUCCUUUU |
| 9 | 350 | AAGGAGUCAGACAUUUUAAUU | 364 | UUAaAaUgUCUGACUCCUUUU |
| 10 | 351 | AAGGAGUCAGACAUUUUAAUU | 365 | UUAAAAUGUCUGACUCCUUUU |
| 11 | 352 | AAGGAGUCAGACAUUUUAAUU | 366 | UUAAAAUGUCUGACUCCUUUU |
| 12 | 353 | AAGGAGUCAGACAUUUUAAUU | 367 | UUAAAAUGUCUGACUCCUUUU |
| 13 | 354 | AAGGAGUCAGACAUUUUAAUU | 368 | UUAAAAUGUCUGACUCCUUUU |

Key for Table 8: Upper case A, G, C and U refer to ribo-A, ribo-G, ribo-C and ribo-U, respectively. The lower case letters a, u, g, c, t refer to 2′-deoxy-A, 2′-deoxy-U, 2′-deoxy-G, 2′-deoxy-C, and deoxythymidine (dT=T=t) respectively.

Underlining refers to 2′-OMe-substituted, e.g., U. N is A, C, G, U, U, a, c, g, u, t, or a modified, inverted, or chemically modified nucleotide.

Embodiments of this invention encompass siRNA molecules of Tables 1-8 that are modified or chemically-modified according to the examples above.

In some embodiments, this invention provides a range of nucleic acid molecules, where a) the molecule has a polynucleotide sense strand and a polynucleotide antisense strand; b) each strand of the molecule is from 15 to 30 nucleotides in length; c) a contiguous region of from 15 to 30 nucleotides of the antisense strand is complementary to a sequence of an mRNA encoding p21; and d) at least a portion of the sense strand is complementary to at least a portion of the antisense strand, and the molecule has a duplex region of from 15 to 30 nucleotides in length.

In some embodiments, the nucleic acid molecule can have a contiguous region of from 15 to 30 nucleotides of the antisense strand that is complementary to a sequence of an mRNA encoding p21, and is located in the duplex region of the molecule.

In additional embodiments, the nucleic acid molecule can have a contiguous region of from 15 to 30 nucleotides of the antisense strand that is complementary to a sequence of an mRNA encoding p21.

In further aspects, a nucleic acid molecule of this invention can have each strand of the molecule being from 18 to 22 nucleotides in length. A nucleic acid molecule can have a duplex region of 19 nucleotides in length.

In certain embodiments, a nucleic acid molecule can have a polynucleotide sense strand and the polynucleotide antisense strand that are connected as a single strand, and form a duplex region connected at one end by a loop.

The nucleic acid molecules of this invention can have a blunt end, and can have one or more 3′ overhangs.

The nucleic acid molecules of this invention can be RNAi molecules that are active for gene silencing, for example, a dsRNA that is active for gene silencing, a siRNA, a micro-RNA, or a shRNA active for gene silencing, as well as a DNA-directed RNA (ddRNA), a Piwi-interacting RNA (piRNA), and a repeat associated siRNA (rasiRNA).

This invention provides a range of nucleic acid molecules that are active for inhibiting expression of p21. In some embodiments, the nucleic acid molecule can have an IC50 for knockdown of p21 of less than 100 μM.

In additional embodiments, the nucleic acid molecule can have an IC50 for knockdown of p21 of less than 50 μM.

This invention further contemplates compositions containing one or more inventive nucleic acid molecules and a pharmaceutically acceptable carrier. The carrier can be a lipid molecule or liposome.

The compounds and compositions of this invention are useful in methods for preventing or treating a p21 associated disease, by administering a compound or composition to a subject in need.

In further aspects, this invention includes methods for treating a disease associated with p21 expression, by administering to a subject in need a composition containing one or more inventive nucleic acid molecules. The disease can be malignant tumor, which may be presented in a disease such as cancers associated with p21 expression, among others.

The methods of this invention can utilize the inventive compounds for preventing or treating malignant tumor. The malignant tumor can be presented in various diseases, for example, cancers associated with p21 expression, cancers caused by cells expressing mutated KRAS, sarcomas, fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, osteosarcoma, carcinomas, brain tumor, head and neck cancer, breast cancer, lung cancer, esophageal cancer, stomach cancer, duodenal cancer, appendix cancer, colorectal cancer, rectal cancer, liver cancer, pancreatic cancer, gallbladder cancer, bile duct cancer, kidney cancer, urethral cancer, urinary bladder cancer, prostate cancer, testicular cancer, uterine cancer, ovary cancer, skin cancer, leukemia, malignant lymphoma, epithelial malignant tumors, and non-epithelial malignant tumors.

Embodiments of this invention can provide RNAi molecules that can be used to down regulate or inhibit the expression of a gene and/or the protein.

The RNAi molecules of this disclosure can be used individually, or in combination with other siRNAs for modulating the expression of one or more genes.

The RNAi molecules of this disclosure can be used individually, or in combination, or in conjunction with other known drugs for preventing or treating diseases, or ameliorating symptoms of conditions or disorders.

The RNAi molecules of this invention can be used to modulate or inhibit the expression of a gene in a sequence-specific manner.

The RNAi molecules of this disclosure can include a guide strand for which a series of contiguous nucleotides are at least partially complementary to a human mRNA.

Embodiments of this invention may include methods for preventing, treating, or ameliorating the symptoms of a disease or condition in a subject in need thereof.

In some embodiments, methods for preventing, treating, or ameliorating the symptoms of malignant tumor in a subject can include administering to the subject a RNAi molecule of this invention to modulate the expression of a gene in the subject or organism.

In some embodiments, this invention contemplates methods for down regulating the expression of a gene in a cell or organism, by contacting the cell or organism with a RNAi molecule of this invention.

### RNAi Molecules Targeted for Hsp47

In some embodiments, this invention can provide a range of RNAi molecules and compositions for modulating expression of heat shock protein 47 (Hsp47), a collagen-specific molecular chaperone for intracellular transport and maturation.

Some examples of siRNAs for Hsp47 are given in U.S. Pat. No. 8,710,209, which is hereby incorporated by reference in its entirety for all purposes.

Hsp47 or a homologous gene sequence thereof is disclosed as, for example, GenBank accession No. AB010273 (human), X60676 (mouse), or M69246 (rat, gp46).

Agents for suppressing Hsp47 have been disclosed for inhibiting fibrosis. See, e.g., U.S. Pat. No. 8,173,170 B2, which is hereby incorporated by reference in its entirety for all purposes. However, limited information exists concerning the effect of inhibiting Hsp47 in malignant tumor development, progression, and growth.

In some embodiments, each strand of a siRNA molecule of this invention can be from 15 to 60 nucleotides in length, or from 15 to 40 nucleotides in length, or from 19 to 25 nucleotides in length.

In certain embodiments, this invention provides a pharmaceutical composition containing RNAi molecules for treating malignant tumor that are RNAi molecules targeted to Hsp47.

Examples of RNAi molecules of this disclosure targeted to Hsp47 mRNA are shown in Table 9.

TABLE 9
RNAi molecule sequences for Hsp47

| | SEQ | SENSE STRAND | SEQ | ANTISENSE STRAND |
|---|---|---|---|---|
| | ID | (5′-->3′) | ID | (5′-->3′) |
| | NO | SEQ ID NOS: 369 to 389 | NO | SEQ ID NOS: 390 to 410 |
| mouse | 369 | CGAGAACAGUUUGUACAAGUU | 390 | CUUGUACAAACUGUUCUCGUU |
| | 370 | CAGGCCUCUACAACUACUATT | 391 | UAGUAGUUGUAGAGGCCUGTT |
| | 371 | GAGCACUCCAAGAUCAACUUC | 392 | CGCGGAAGUUGAUCUUGGAGU |
| | | CGCG | | GCUCUU |
| | 372 | GGACAGGCCUCUACAACUATT | 393 | UAGUUGUAGAGGCCUGUCCTT |
| | 373 | GAGCACUCCAAGAUCAACUTT | 394 | AGUUGAUCUUGGAGUGCUCTT |
| | 374 | GAACACUCCAAGAUCAACUTT | 395 | AGUUGAUCUUGGAGUGUUCTT |
| | 375 | CAGGCCUCUACAACUACUACG | 396 | UCGUCGUAGUAGUUGUAGAGG |
| | | ACGA | | CCUGUU |
| | 376 | GAACACUCCAAGAUCAACUUCC | 397 | CUCGGAAGUUGAUCUUGGAGU |
| | | GAG | | GUUCUU |
| | 377 | GGACAGGCCUCUACAACUACU | 398 | UCGUAGUAGUUGUAGAGGCCU |
| | | ACGA | | GUCCUU |
| | 378 | CAGGCCUCUACAACUACUAdTd | 399 | UAGUAGUUGUAGAGGCCUGdT |
| | | TdAdAdAdAdA | | dT |
| | 379 | CAGGCCUCUACAACUACUA | 400 | UAGUAGUUGUAGAGGCCUGdT |
| | | | | dT |
| | 380 | CAGGCCUCUACAACUACUAdTdT | 401 | UAGUAGUUGUAGAGGCCUGdT |
| | | dAdAdAdAdAdAdAdAdAdAdAdA | | dT |
| | 381 | dAdAdAdAdACAGGCCUCUACAA | 402 | UAGUAGUUGUAGAGGCCUGdT |
| | | CUACUAdTdT | | dT |
| | 382 | CAGGCCUCUACAACUACUAdTd | 403 | UAGUAGUUGUAGAGGCCUGdT |
| | | TdAdAdAdAdAdAdAdA | | dT |
| | 383 | dAdAdAdAdAdAdAdACAGGCCU | 404 | UAGUAGUUGUAGAGGCCUGdT |
| | | CUACAACUACUAdTdT | | dT |
| | 384 | CAGGCCUCUACAACUACUAdTdTd | 405 | UAGUAGUUGUAGAGGCCUGdT |
| | | AdAdAdAdAdAdAdAdAdAdAdA | | dT |
| | 385 | dAdAdAdAdAdAdAdAdAdAdAdA | 406 | UAGUAGUUGUAGAGGCCUGdT |
| | | dAdAdAdACAGGCCUCUACAACU | | dT |
| | | ACUAdTdT | | |
| | 386 | CAGGCCUCUACAACUACUAdTdTd | 407 | UAGUAGUUGUAGAGGCCUGdT |
| | | AdAdAdAdAdAdAdAdAdAdAdA | | dT |
| | | dAdAdAdA | | |
| | 387 | CAGGCCUCUACAACUACUAdTd | 408 | UAGUAGUUGUAGAGGCCUGdT |
| | | T | | dT |
| mouse | 388 | GGACAGGCCUGUACAACUAdTd | 409 | UAGUUGUACAGGCCUGUCCdTd |
| | | T | | T |
| human | 389 | GGACAGGCCUCUACAACUAdTd | 410 | UAGUUGUAGAGGCCUGUCCdT |
| | | T | | dT |

Key for Table 9: Upper case A, G, C and U referred to for ribo-A, ribo-G, ribo-C and ribo-U respectively. Lower case d represents “deoxy.”

In Table 9, the antisense strand of any of the RNAi molecules targeted to Hsp47 may alternatively have deoxynucleotides in a plurality of positions, the plurality of positions being one of the following: each of positions 4, 6 and 8, from the 5′ end of the antisense strand; each of positions 3, 5 and 7, from the 5′ end of the antisense strand; each of positions 1, 3, 5 and 7, from the 5′ end of the antisense strand; each of positions 3-8, from the 5′ end of the antisense strand; and each of positions 5-8, from the 5′ end of the antisense strand.

Additional examples of RNAi molecules of this disclosure targeted to Hsp47 mRNA are shown in Table 10.

TABLE 10
RNAi molecule sequences and control for Hsp47

| SEQ | | |
|---|---|---|
| ID | | |
| NO | SEQUENCE | Name |
| 411 | 3′-C3-25rU-25rC-25rC-25rU-25rU-rC-rA- | SENSE “A” |
| | rA-rC-rU-rA-rG-rA-rA-rC-rC-rU-rC-rA- | |
| | idAB-5′ | |
| 412 | 3′-C3-C3-mU-rG-mA-rG-mG-rU-mU-rC-mU- | ANTISENSE “A” |
| | rA-rG-mU-25rU-mG-rA-mA-rG-mG-rA-5′ | |
| 413 | 3′-C3-rA-rG-mU-rG-rG-rG-mU-rA-mC-rA- | SENSE “B” |
| | 25rC-rA-rG-rA-rG-mU-rC-rC-rU-idAB-5′ | |
| 414 | 3′-C3-C3-rA-rG-rG-rA-mC-rU-rC-rU-mG- | ANTISENSE “B” |
| | rU-rG-mU-rA-25rC-rC-mC-rA-rC-mU-5′ | |
| 415 | 5′-idAB-rG-rA-rG-rA-rC-rA-rC-rA-rU-rG- | SENSE “C” |
| | rG-rG-rU-rG-25rC-25rU-25rA-25rU-25rA- | |
| | C3-P-3′ | |
| 416 | 5′-mU-rA-mU-rA-mG-rC-25rA-rC-mC-rC- | ANTISENSE “C” |
| | mA-rU-mG-rU-mG-rU-mC-rU-mC-C3-C3-3′ | |
| 417 | 5′-idAB-rC-mU-mU-rA-mC-rG-mC-mU- | SENSE “D” |
| | 25rG-rA-rG-mU-rA-mC-mU-rU-mC-rG-rU- | Negative control |
| | C3-3′ | |
| 418 | 5′-rA-mC-rG-rA-rA-25rG-mU-rA-rC-rU- | ANTISENSE “D” |
| | mC-rA-rG-rC-rG-mU-rA-rA-rG-C3-C3-3′ | Negative control |

Key for Table 10: Designations: rX represents ribonucleotides, mX represents 2′-O-Methyl ribonucleotides, 25rX represents ribonucleotides with 2′-5′ linkages, C3 represents a 1,3-propanediol spacer, idAB represents inverted 1,2-dideoxy-D-Ribose, P represents a phosphate group on the 3′-terminus.

In Table 10, the antisense strand of any of the RNAi molecules targeted to Hsp47 may alternatively have deoxynucleotides in a plurality of positions, the plurality of positions being one of the following: each of positions 4, 6 and 8, from the 5′ end of the antisense strand; each of positions 3, 5 and 7, from the 5′ end of the antisense strand;

each of positions 1, 3, 5 and 7, from the 5′ end of the antisense strand; each of positions 3-8, from the 5′ end of the antisense strand; and each of positions 5-8, from the 5′ end of the antisense strand.

### RNAi Molecules Targeted for MCL1

In some embodiments, this invention can provide a range of RNAi molecules and compositions for modulating expression of gene Homo sapiens myeloid cell leukemia 1 (MCL1), transcript variant 2, mRNA.

MCL1 is disclosed at, for example, Accession No. NM 182763.2 (human).

Examples of RNAi molecules of this disclosure targeted to MCL1 mRNA are shown in Table 11.

TABLE 11
RNAi molecule sequences for MCL1

| SEQ | SENSE STRAND | SEQ | ANTISENSE STRAND |
|---|---|---|---|
| ID | (5′-->3′) | ID | (5′-->3′) |
| NO | SEQ ID NOS: 419 to 426 | NO | SEQ ID NOS: 427 to 434 |
| 419 | GCCUUCCAAGGAUGGGUUUGU | 427 | AAACCCAUCCUUGGAAGGCCG |
| 420 | GGAGUUCUUCCAUGUAGAGGA | 428 | CUCUACAUGGAAGAACUCCAC |
| 421 | CCAUGUAGAGGACCUAGAAGG | 429 | UUCUAGGUCCUCUACAUGGAA |
| 422 | GCCUUCCAAGGAUGGGUUUGU | 430 | AAAcCcAuCCUUGGAAGGCCG |
| 423 | GCCUUCCAAGGAUGGGUUUGU | 431 | AAaCcCaUCCUUGGAAGGCCG |
| 424 | GCCUUCCAAGGAUGGGUUUGU | 432 | aAaCcCaUCCUUGGAAGGCCG |
| 425 | GCCUUCCAAGGAUGGGUUUGU | 433 | AAacccauCCUUGGAAGGCCG |
| 426 | GCCUUCCAAGGAUGGGUUUGU | 434 | AAACccauCCUUGGAAGGCCG |

Key for Table 11: Upper case A, G, C and U referred to for ribo-A, ribo-G, ribo-C and ribo-U respectively. Lower case d represents “deoxy.”

In Table 11, the antisense strand of any of the RNAi molecules targeted to MCL1 may alternatively have deoxynucleotides in a plurality of positions, the plurality of positions being one of the following: each of positions 4, 6 and 8, from the 5′ end of the antisense strand; each of positions 3, 5 and 7, from the 5′ end of the antisense strand; each of positions 1, 3, 5 and 7, from the 5′ end of the antisense strand; each of positions 3-8, from the 5′ end of the antisense strand; and each of positions 5-8, from the 5′ end of the antisense strand.

### RNAi Molecules Targeted for ARAF

In some embodiments, this invention can provide a range of RNAi molecules and compositions for modulating expression of gene Homo sapiens A-Raf proto-oncogene, serine/threonine kinase (ARAF), transcript variant 1, mRNA.

ARAF is disclosed at, for example, Accession No. NM 001654.4 (human).

Examples of RNAi molecules of this disclosure targeted to ARAF mRNA are shown in Table 12.

TABLE 12
RNAi molecule sequences for ARAF

| SEQ | SENSE STRAND | SEQ | ANTISENSE STRAND |
|---|---|---|---|
| ID | (5′-->3′) | ID | (5′-->3′) |
| NO | SEQ ID NOS: 435 to 442 | NO | SEQ ID NOS: 443 to 450 |
| 435 | GCUCAUUGUCGAGGUCCUUGA | 443 | AAGGACCUCGACAAUGAGCUC |
| 436 | GCCAAACCUGUGGCUACAAGU | 444 | UUGUAGCCACAGGUUUGGCAA |
| 437 | GGAAGACGCGACAUGUCAACA | 445 | UUGACAUGUCGCGUCUUCCUG |
| 438 | GCUCAUUGUCGAGGUCCUUGA | 446 | AAGgAcCuCGACAAUGAGCUC |
| 439 | GCUCAUUGUCGAGGUCCUUGA | 447 | AAgGaCcUCGACAAUGAGCUC |
| 440 | GCUCAUUGUCGAGGUCCUUGA | 448 | aAgGaCcUCGACAAUGAGCUC |
| 441 | GCUCAUUGUCGAGGUCCUUGA | 449 | AAggaccuCGACAAUGAGCUC |
| 442 | GCUCAUUGUCGAGGUCCUUGA | 450 | AAGGaccuCGACAAUGAGCUC |

Key for Table 12: Upper case A, G, C and U referred to for ribo-A, ribo-G, ribo-C and ribo-U respectively. Lower case d represents “deoxy.”

In Table 12, the antisense strand of any of the RNAi molecules targeted to ARAF may alternatively have deoxynucleotides in a plurality of positions, the plurality of positions being one of the following: each of positions 4, 6 and 8, from the 5′ end of the antisense strand; each of positions 3, 5 and 7, from the 5′ end of the antisense strand; each of positions 1, 3, 5 and 7, from the 5′ end of the antisense strand; each of positions 3-8, from the 5′ end of the antisense strand; and each of positions 5-8, from the 5′ end of the antisense strand.

### RNA Interference

In general, siRNAs can be from about 21 to about 23 nucleotides in length and include a base pair duplex region about 19 nucleotides in length.

RNAi involves an endonuclease complex known as the RNA induced silencing complex (RISC). An siRNA has an antisense or guide strand which enters the RISC complex and mediates cleavage of a single stranded RNA target having a sequence complementary to the antisense strand of the siRNA duplex. The other strand of the siRNA is the passenger strand. Cleavage of the target RNA takes place in the middle of the region complementary to the antisense strand of the siRNA duplex See, e.g., Elbashir et al., Genes & Development, 2001, Vol. 15, pp. 188-200.

As used herein, the term “sense strand” refers to a nucleotide sequence of a siRNA molecule that is partially or fully complementary to at least a portion of a corresponding antisense strand of the siRNA molecule. The sense strand of a siRNA molecule can include a nucleic acid sequence having homology with a target nucleic acid sequence.

As used herein, the term “antisense strand” refers to a nucleotide sequence of a siRNA molecule that is partially or fully complementary to at least a portion of a target nucleic acid sequence. The antisense strand of a siRNA molecule can include a nucleic acid sequence that is complementary to at least a portion of a corresponding sense strand of the siRNA molecule.

RNAi molecules can down regulate or knock down gene expression by mediating RNA interference in a sequence-specific manner. See, e.g., Zamore et al., Cell, 2000, Vol. 101, pp. 25-33; Elbashir et al., Nature, 2001, Vol. 411, pp. 494-498; Kreutzer et al., WO2000/044895; Zernicka-Goetz et al., WO2001/36646; Fire et al., WO1999/032619; Plaetinck et al., WO2000/01846; Mello et al., WO2001/029058.

As used herein, the terms “inhibit,” “down-regulate,” or “reduce” with respect to gene expression means that the expression of the gene, or the level of mRNA molecules encoding one or more proteins, or the activity of one or more of the encoded proteins is reduced below that observed in the absence of a RNAi molecule or siRNA of this invention. For example, the level of expression, level of mRNA, or level of encoded protein activity may be reduced by at least 1%, or at least 10%, or at least 20%, or at least 50%, or at least 90%, or more from that observed in the absence of a RNAi molecule or siRNA of this invention.

RNAi molecules can also be used to knock down viral gene expression, and therefore affect viral replication.

RNAi molecules can be made from separate polynucleotide strands: a sense strand or passenger strand, and an antisense strand or guide strand. The guide and passenger strands are at least partially complementary. The guide strand and passenger strand can form a duplex region having from about 15 to about 49 base pairs.

In some embodiments, the duplex region of a siRNA can have 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 base pairs.

In certain embodiments, a RNAi molecule can be active in a RISC complex, with a length of duplex region active for RISC.

In additional embodiments, a RNAi molecule can be active as a Dicer substrate, to be converted to a RNAi molecule that can be active in a RISC complex.

In some aspects, a RNAi molecule can have complementary guide and passenger sequence portions at opposing ends of a long molecule, so that the molecule can form a duplex region with the complementary sequence portions, and the strands are linked at one end of the duplex region by either nucleotide or non-nucleotide linkers. For example, a hairpin arrangement, or a stem and loop arrangement. The linker interactions with the strands can be covalent bonds or non-covalent interactions.

A RNAi molecule of this disclosure may include a nucleotide, non-nucleotide, or mixed nucleotide/non-nucleotide linker that joins the sense region of the nucleic acid to the antisense region of the nucleic acid. A nucleotide linker can be a linker of ≥2 nucleotides in length, for example about 3, 4, 5, 6, 7, 8, 9, or 10 nucleotides in length. The nucleotide linker can be a nucleic acid aptamer. By “aptamer” or “nucleic acid aptamer” as used herein refers to a nucleic acid molecule that binds specifically to a target molecule wherein the nucleic acid molecule has sequence that includes a sequence recognized by the target molecule in its natural setting. Alternately, an aptamer can be a nucleic acid molecule that binds to a target molecule, where the target molecule does not naturally bind to a nucleic acid. For example, the aptamer can be used to bind to a ligand-binding domain of a protein, thereby preventing interaction of the naturally occurring ligand with the protein. See, e.g., Gold et al., Annu Rev Biochem, 1995, Vol. 64, pp. 763-797; Brody et al., J. Biotechnol., 2000, Vol. 74, pp. 5-13; Hermann et al., Science, 2000, Vol. 287, pp. 820-825.

Examples of a non-nucleotide linker include an abasic nucleotide, polyether, polyamine, polyamide, peptide, carbohydrate, lipid, polyhydrocarbon, or other polymeric compounds, for example polyethylene glycols such as those having from 2 to 100 ethylene glycol units. Some examples are described in Seela et al., Nucleic Acids Research, 1987, Vol. 15, pp. 3113-3129; Cload et al., J. Am. Chem. Soc., 1991, Vol. 113, pp. 6324-6326; Jaeschke et al., Tetrahedron Lett., 1993, Vol. 34, pp. 301; Arnold et al., WO1989/002439; Usman et al., WO1995/006731; Dudycz et al., WO1995/011910, and Ferentz et al., J. Am. Chem. Soc., 1991, Vol. 113, pp. 4000-4002.

A RNAi molecule can have one or more overhangs from the duplex region. The overhangs, which are non-base-paired, single strand regions, can be from one to eight nucleotides in length, or longer. An overhang can be a 3′-end overhang, wherein the 3′-end of a strand has a single strand region of from one to eight nucleotides. An overhang can be a 5′-end overhang, wherein the 5′-end of a strand has a single strand region of from one to eight nucleotides.

The overhangs of a RNAi molecule can have the same length, or can be different lengths.

A RNAi molecule can have one or more blunt ends, in which the duplex region ends with no overhang, and the strands are base paired to the end of the duplex region.

A RNAi molecule of this disclosure can have one or more blunt ends, or can have one or more overhangs, or can have a combination of a blunt end and an overhang end.

A 5′-end of a strand of a RNAi molecule may be in a blunt end, or can be in an overhang. A 3′-end of a strand of a RNAi molecule may be in a blunt end, or can be in an overhang.

A 5′-end of a strand of a RNAi molecule may be in a blunt end, while the 3′-end is in an overhang. A 3′-end of a strand of a RNAi molecule may be in a blunt end, while the 5′-end is in an overhang.

In some embodiments, both ends of a RNAi molecule are blunt ends.

In additional embodiments, both ends of a RNAi molecule have an overhang.

The overhangs at the 5′- and 3′-ends may be of different lengths.

In certain embodiments, a RNAi molecule may have a blunt end where the 5′-end of the antisense strand and the 3′-end of the sense strand do not have any overhanging nucleotides.

In further embodiments, a RNAi molecule may have a blunt end where the 3′-end of the antisense strand and the 5′-end of the sense strand do not have any overhanging nucleotides.

A RNAi molecule may have mismatches in base pairing in the duplex region.

Any nucleotide in an overhang of a RNAi molecule can be a deoxyribonucleotide, or a ribonucleotide.

One or more deoxyribonucleotides may be at the 5′-end, where the 3′-end of the other strand of the RNAi molecule may not have an overhang, or may not have a deoxyribonucleotide overhang.

One or more deoxyribonucleotides may be at the 3′-end, where the 5′-end of the other strand of the RNAi molecule may not have an overhang, or may not have a deoxyribonucleotide overhang.

In some embodiments, one or more, or all of the overhang nucleotides of a RNAi molecule may be 2′-deoxyribonucleotides.

### Dicer Substrate RNAi Molecules

In some aspects, a RNAi molecule can be of a length suitable as a Dicer substrate, which can be processed to produce a RISC active RNAi molecule. See, e.g., Rossi et al., US2005/0244858.

A double stranded RNA (dsRNA) that is a Dicer substrate can be of a length sufficient such that it is processed by Dicer to produce an active RNAi molecule, and may further include one or more of the following properties: (i) the Dicer substrate dsRNA can be asymmetric, for example, having a 3′ overhang on the antisense strand, and (ii) the Dicer substrate dsRNA can have a modified 3′ end on the sense strand to direct orientation of Dicer binding and processing of the dsRNA to an active RNAi molecule.

In certain embodiments, the longest strand in a Dicer substrate dsRNA may be 24-30 nucleotides in length.

A Dicer substrate dsRNA can be symmetric or asymmetric.

In some embodiments, a Dicer substrate dsRNA can have a sense strand of 22-28 nucleotides and an antisense strand of 24-30 nucleotides.

In certain embodiments, a Dicer substrate dsRNA may have an overhang on the 3′ end of the antisense strand.

In further embodiments, a Dicer substrate dsRNA may have a sense strand 25 nucleotides in length, and an antisense strand 27 nucleotides in length, with a 2 base 3′-overhang. The overhang may be 1, 2 or 3 nucleotides in length. The sense strand may also have a 5′ phosphate.

An asymmetric Dicer substrate dsRNA may have two deoxyribonucleotides at the 3′-end of the sense strand in place of two of the ribonucleotides.

The sense strand of a Dicer substrate dsRNA may be from about 22 to about 30, or from about 22 to about 28; or from about 24 to about 30; or from about 25 to about 30; or from about 26 to about 30; or from about 26 and 29; or from about 27 to about 28 nucleotides in length.

The sense strand of a Dicer substrate dsRNA may be 22, 23, 24, 25, 26, 27, 28, 29 or 30 nucleotides in length.

In certain embodiments, a Dicer substrate dsRNA may have sense and antisense strands that are at least about 25 nucleotides in length, and no longer than about 30 nucleotides in length.

In certain embodiments, a Dicer substrate dsRNA may have sense and antisense strands that are 26 to 29 nucleotides in length.

In certain embodiments, a Dicer substrate dsRNA may have sense and antisense strands that are 27 nucleotides in length.

The sense and antisense strands of a Dicer substrate dsRNA may be the same length as in being blunt ended, or different lengths as in having overhangs, or may have a blunt end and an overhang.

A Dicer substrate dsRNA may have a duplex region of 19, 20, 21, 22, 23, 24, 25, 26 or 27 nucleotides in length.

The antisense strand of a Dicer substrate dsRNA may have any sequence that anneals to at least a portion of the sequence of the sense strand under biological conditions, such as within the cytoplasm of a eukaryotic cell.

A Dicer substrate with a sense and an antisense strand can be linked by a third structure, such as a linker group or a linker oligonucleotide. The linker connects the two strands of the dsRNA, for example, so that a hairpin is formed upon annealing.

The sense and antisense strands of a Dicer substrate are in general complementary, but may have mismatches in base pairing.

In some embodiments, a Dicer substrate dsRNA can be asymmetric such that the sense strand has 22-28 nucleotides and the antisense strand has 24-30 nucleotides.

A region of one of the strands, particularly the antisense strand, of the Dicer substrate dsRNA may have a sequence length of at least 19 nucleotides, wherein these nucleotides are in the 21-nucleotide region adjacent to the 3′ end of the antisense strand and are sufficiently complementary to a nucleotide sequence of the RNA produced from the target gene.

An antisense strand of a Dicer substrate dsRNA can have from 1 to 9 ribonucleotides on the 5′-end, to give a length of 22-28 nucleotides. When the antisense strand has a length of 21 nucleotides, then 1-7 ribonucleotides, or 2-5 ribonucleotides, or 4 ribonucleotides may be added on the 3′-end. The added ribonucleotides may have any sequence.

A sense strand of a Dicer substrate dsRNA may have 24-30 nucleotides. The sense strand may be substantially complementary with the antisense strand to anneal to the antisense strand under biological conditions.

### Methods for Using RNAi Molecules

The nucleic acid molecules and RNAi molecules of this invention may be delivered to a cell or tissue by direct application of the molecules, or with the molecules combined with a carrier or a diluent.

The nucleic acid molecules and RNAi molecules of this invention can be delivered or administered to a cell, tissue, organ, or subject by direct application of the molecules with a carrier or diluent, or any other delivery vehicle that acts to assist, promote or facilitate entry into a cell, for example, viral sequences, viral material, or lipid or liposome formulations.

The nucleic acid molecules and RNAi molecules of this invention can be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection.

Delivery systems may include, for example, aqueous and nonaqueous gels, creams, emulsions, microemulsions, liposomes, ointments, aqueous and nonaqueous solutions, lotions, aerosols, hydrocarbon bases and powders, and can contain excipients such as solubilizers and permeation enhancers.

Compositions and methods of this disclosure can include an expression vector that includes a nucleic acid sequence encoding at least one RNAi molecule of this invention in a manner that allows expression of the nucleic acid molecule.

The nucleic acid molecules and RNAi molecules of this invention can be expressed from transcription units inserted into DNA or RNA vectors. Recombinant vectors can be DNA plasmids or viral vectors. Viral vectors can be used that provide for transient expression of nucleic acid molecules.

For example, the vector may contain sequences encoding both strands of a RNAi molecule of a duplex, or a single nucleic acid molecule that is self-complementary and thus forms a RNAi molecule. An expression vector may include a nucleic acid sequence encoding two or more nucleic acid molecules.

A nucleic acid molecule may be expressed within cells from eukaryotic promoters. Those skilled in the art realize that any nucleic acid can be expressed in eukaryotic cells from the appropriate DNA/RNA vector.

In some aspects, a viral construct can be used to introduce an expression construct into a cell, for transcription of a dsRNA construct encoded by the expression construct.

Lipid formulations can be administered to animals by intravenous, intramuscular, or intraperitoneal injection, or orally or by inhalation or other methods as are known in the art.

Pharmaceutically acceptable formulations for administering oligonucleotides are known and can be used.

### Methods for Treating Disease

Examples of diseases include cancer, sarcomas, fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, osteosarcoma, and carcinomas.

In certain aspects, methods of this invention can utilize the inventive compounds for preventing or treating malignant tumors and cancers in any organ or tissue, including, for example, brain tumor, head and neck cancer, breast cancer, lung cancer, esophageal cancer, stomach cancer, duodenal cancer, colorectal cancer, liver cancer, pancreatic cancer, gallbladder cancer, bile duct cancer, kidney cancer, urethral cancer, bladder cancer, prostate cancer, testicular cancer, uterine cancer, ovary cancer, skin cancer, leukemia, malignant lymphoma, epithelial malignant tumors, and non-epithelial malignant tumors.

### Example Protocol for In Vitro Knockdown

One day before the transfection, cells were plated in a 96-well plate at 2×10³ cells per well with 100 μl of DMEM (HyClone Cat. #SH30243.01) containing 10% FBS and culture in a 37° C. incubator containing a humidified atmosphere of 5% CO₂ in air. Before transfection, medium was changed to 90 μl of Opti-MEM I Reduced Serum Medium (Life Technologies Cat. #31985-070) containing 2% FBS. Then, 0.2 μl of Lipofectamine RNAiMax (Life Technologies Cat. #13778-100) was mixed with 4.8 μl of Opti-MEM I for 5 minutes at room temperature. Next, 1 μl of siRNA was mixed with 4 μl of Opti-MEM I and combined with the LF2000 solution, and mixed gently, without vortex. After 5 minutes at room temperature, the mixture was incubated for an additional 10 minutes at room temperature to allow the RNA-RNAiMax complexes to form. Further, the 10 μl of RNA-RNAiMax complexes was added to a well, and the plate was shaken gently by hand. The cells were incubated in a 37° C. incubator containing a humidified atmosphere of 5% CO₂ in air for 2 hours. The medium was changed to fresh Opti-MEM I Reduced Serum Medium containing 2% FBS. 24 hours after transfection, the cells were washed with ice-cold PBS once. The cells were lysed with 50 μl of Cell-to-Ct Lysis Buffer (Life Technologies Cat. #4391851 C) for 5-30 minutes at room temperature. 5 μl of Stop Solution was added, and it was incubated for 2 minutes at room temperature. The mRNA level was measured by RT-qPCR with TAQMAN immediately. Samples could be frozen at −80° C. and assayed at a later time.

### Example Protocol for Serum Stability

0.2 mg/ml siRNA was incubated with 10% human serum at 37° C. At certain time points (0, 5, 15 and 30 min), 200 μl of sample was aliquoted and extracted with 200 μl extraction solvent (Chloroform:phenol:Isoamyl alcohol=24:25:1). The sample was vortexed and centrifuged at 13,000 rpm for 10 min at RT, then the top layer solution was transferred and filtered it with 0.45 μm filter. The filtrate was transferred into a 300 μl HPLC injection vial. For LCMS, the Mobile phase was MPA: 100 mM HFIP+7 mM TEA in H₂O, MPB: 50% Methanol+50% Acetonitrile. The Column: Waters Acquity OST 2.1×50 mm, 1.7 μm.

### EXAMPLES

Example 1: siRNAs of this invention targeted to GST-π were found to be active for gene silencing in vitro. The dose-dependent activities of GST-π siRNAs for gene knockdown were found to exhibit an IC50 below about 250 picomolar (pM), and as low as 1 μM.

In vitro transfection was performed in an A549 cell line to determine siRNA knockdown efficacy. Dose dependent knockdown for GST-π mRNA was observed with siRNAs of Table 1, as shown in Table 13.

TABLE 13
Dose dependent knockdown for GST-π mRNA in an A549 cell line

| siRNA structure | IC50 (pM) |
|---|---|
| A9 (SEQ ID NOs: 25 and 90) | 24 |
| B2 (SEQ ID NOs: 52 and 117) | 121 |
| B3 (SEQ ID NOs: 53 and 118) | 235 |
| B4 (SEQ ID NOs: 54 and 119) | 229 |
| B13 (SEQ ID NOs: 50 and 115) | 17 |
| BU2 (SEQ ID NOs: 61 and 126) | 31 |

As shown in Table 13, the activities of GST-π siRNAs of Table 1 were in the range 17-235 μM, which is suitable for many uses, including as a drug agent to be used in vivo.

Example 2: The structure of GST-π siRNAs of this invention having deoxynucleotides located in the seed region of the antisense strand of the siRNA provided unexpectedly and advantageously increased gene knockdown activity in vitro.

In vitro transfection was performed in an A549 cell line to determine knockdown efficacy for GST-π siRNAs based on structure BU2′ (SEQ ID NOs: 131 and 157). Dose dependent knockdown of GST-π mRNA was observed with GST-π siRNAs based on structure BU2′ as shown in Table 14.

TABLE 14
Dose dependent knockdown of GST-π mRNA in an A549 cell
line for GST-π siRNAs based on structure BU2′

| GST-π siRNA structure | IC50 (pM) |
|---|---|
| BU2 with no deoxynucleotides in the duplex region | 31 |
| (SEQ ID NOs: 61 and 126) | |
| BU2 with deoxynucleotides in positions 3, 5, and 7 of | 5 |
| the seed region antisense strand (SEQ ID NOs: 139 | |
| and 165) | |
| BU2 with deoxynucleotides in positions 4, 6, and 8 of | 8 |
| the seed region antisense strand (SEQ ID NOs: 141 | |
| and 167) | |
| BU2 with deoxynucleotides in positions 4, 6, and 8 of | 5 |
| the seed region antisense strand (SEQ ID NOs: 156 | |
| and 182) | |

As shown in Table 14, the activities of GST-π siRNAs based on structure BU2′ having three deoxynucleotides in the seed region of the antisense strand were surprisingly and unexpectedly increased by up to 6-fold, as compared to a GST-π siRNA without deoxynucleotides in the duplex region.

These data show that GST-π siRNAs having a structure with three deoxynucleotides located at positions 3, 5 and 7, or at positions 4, 6 and 8 in the seed region of the antisense strand provided surprisingly increased gene knockdown activity as compared to a GST-π siRNA without deoxynucleotides in the duplex region.

The activities shown in Table 14 for GST-π siRNAs having three deoxynucleotides in the seed region of the antisense strand were in the range 5 to 8 μM, which is exceptionally suitable for many uses, including as a drug agent to be used in vivo.

Example 3: The structure of GST-π siRNAs of this invention having deoxynucleotides located in the seed region of the antisense strand of the siRNA provided unexpectedly and advantageously increased gene knockdown activity in vitro.

In vitro transfection was performed in an A549 cell line to determine knockdown efficacy for GST-π siRNAs based on structure A9′ (SEQ ID NOs: 183 and 195). Dose dependent knockdown of GST-π mRNA was observed with the GST-π siRNAs based on structure A9′, as shown in Table 15.

TABLE 15
Dose dependent knockdown of GST-π mRNA in an A549 cell line for
GST-π siRNAs based on structure structure A9′

| GST-π siRNA structure | IC50 (pM) |
|---|---|
| A9 with no deoxynucleotides in the duplex region | 24 |
| (SEQ ID NOs: 25 and 90) | |
| A9 with deoxynucleotides in positions 4, 6, and 8 of | 1 |
| the seed region antisense strand (SEQ ID NOs: 193 | |
| and 205) | |
| A9 with deoxynucleotides in positions 1, 3, 5, and 7 | 5 |
| of the seed region antisense strand (SEQ ID NOs: 190 | |
| and 202) | |
| A9 with deoxynucleotides in positions 3-8 of the seed | 6 |
| region antisense strand (SEQ ID NOs: 187 and 199) | |
| A9 with deoxynucleotides in positions 5-8 of the seed | 7 |
| region antisense strand (SEQ ID NOs: 189 and 201) | |
| A9 with deoxynucleotides in positions 3, 5, and 7 of | 15 |
| the seed region antisense strand (SEQ ID NOs: 191 | |
| and 203) | |

As shown in Table 15, the activities of GST-π siRNAs based on structure A9′ having three to six deoxynucleotides in the seed region of the antisense strand were surprisingly increased by up to 24-fold, as compared to a GST-π siRNA without deoxynucleotides in the duplex region.

These data show that GST-π siRNAs having a structure with three to six deoxynucleotides located at positions 4, 6 and 8, or at positions 1, 3, 5 and 7, or at positions 3-8, or at positions 5-8, or at positions 3, 5 and 7 in the seed region of the antisense strand provided unexpectedly increased gene knockdown activity as compared to a GST-π siRNA without deoxynucleotides in the duplex region.

The activity shown in Table 15 for GST-π siRNAs having three to six deoxynucleotides in the seed region of the antisense strand was in the range 1 to 15 μM, which is exceptionally suitable for many uses, including as a drug agent to be used in vivo.

Example 4: The structure of GST-π siRNAs having deoxynucleotides located in the seed region of the antisense strand of the siRNA provided unexpectedly and advantageously increased gene knockdown activity in vitro.

In vitro transfection was performed in an A549 cell line to determine knockdown efficacy for GST-π siRNAs based on structure B13′ (SEQ ID NOs: 207 and 222). Dose dependent knockdown of GST-π mRNA was observed with the GST-π siRNAs based on structure B13′, as shown in Table 16.

TABLE 16
Dose dependent knockdown of GST-π mRNA in an A549 cell
line for GST-π siRNAs based on structure B13′

| GST-π siRNA structure | IC50 (pM) |
|---|---|
| B13 with no deoxynucleotides in the duplex region | 17 |
| (SEQ ID NOs: 50 and 115) | |
| B13 with deoxynucleotides in positions 4, 6, and 8 of | 11 |
| the seed region antisense strand (SEQ ID NOs: 217 | |
| and 232) | |

As shown in Table 16, the activity of a GST-π siRNA based on structure B13′ having three deoxynucleotides in the seed region of the antisense strand was unexpectedly increased, as compared to a GST-π siRNA without deoxynucleotides in the duplex region.

These data show that GST-π siRNAs having a structure with three deoxynucleotides located at positions 4, 6 and 8 in the seed region of the antisense strand provided unexpectedly increased gene knockdown activity as compared to a GST-π siRNA without deoxynucleotides in the duplex region.

The activity shown in Table 16 for GST-π siRNAs having three deoxynucleotides in the seed region of the antisense strand was in the picomolar range at 11 μM, which is exceptionally suitable for many uses, including as a drug agent to be used in vivo.

Example 5: The structure of GST-π siRNAs having deoxynucleotides located in the seed region of the antisense strand of the siRNA provided unexpectedly and advantageously increased gene knockdown activity in vitro.

In vitro transfection was performed in an A549 cell line to determine knockdown efficacy for GST-π siRNAs based on structure B4′ (SEQ ID NOs: 261 and 273). Dose dependent knockdown of GST-π mRNA was observed with the GST-π siRNAs based on structure B4′, as shown in Table 17.

TABLE 17
Dose dependent knockdown of GST-π mRNA in an A549 cell line
for GST-π siRNAs based on structure B4′

| GST-π siRNA structure | IC50 (pM) |
|---|---|
| B4 with no deoxynucleotides in the duplex region | 229 |
| (SEQ ID NOs: 54 and 119) | |
| B4 with deoxynucleotides in positions 3-8 of the seed | 113 |
| region antisense strand (SEQ ID NOs: 265 and 277) | |

As shown in Table 17, the activities of GST-π siRNAs based on structure B4′ having six deoxynucleotides in the seed region of the antisense strand were unexpectedly increased by more than two-fold, as compared to a GST-π siRNA without deoxynucleotides in the duplex region.

These data show that GST-π siRNAs having a structure with six deoxynucleotides located at positions 3-8 in the seed region of the antisense strand provided surprisingly increased gene knockdown activity as compared to a GST-π siRNA without deoxynucleotides in the duplex region.

The activity shown in Table 17 for a GST-π siRNA having six deoxynucleotides in the seed region of the antisense strand was in the picomolar range at 113 μM, which is exceptionally suitable for many uses, including as a drug agent to be used in vivo.

Example 6: The structure of GST-π siRNAs having deoxynucleotides located in the seed region of the antisense strand of the siRNA provided unexpectedly and advantageously increased gene knockdown activity in vitro.

In vitro transfection was performed in an A549 cell line to determine knockdown efficacy for GST-π siRNAs based on structure B2′ (SEQ ID NOs: 237 and 249). Dose dependent knockdown of GST-π mRNA was observed with the GST-π siRNAs based on structure B2′, as shown in Table 18.

TABLE 18
Dose dependent knockdown of GST-π mRNA in an A549 cell line
for GST-π siRNAs based on structure B2′

| GST-π siRNA structure | IC50 (pM) |
|---|---|
| B2 with no deoxynucleotides in the duplex regioin | 121 |
| (SEQ ID NOs: 52 and 117) | |
| B2 with deoxynucleotides in positions 5-8 of the seed | 30 |
| region antisense strand (SEQ ID NOs: 243 and 255) | |
| B2 with deoxynucleotides in positions 1, 3, 5, and 7 | 50 |
| of the seed region antisense strand (SEQ ID NOs: 244 | |
| and 256) | |
| B2 with deoxynucleotides in positions 3, 5, and 7 of | 100 |
| the seed region antisense strand (SEQ ID NOs: 245 | |
| and 257) | |

As shown in Table 18, the activities of GST-π siRNAs based on structure B2′ having three to four deoxynucleotides in the seed region of the antisense strand were surprisingly increased by up to 4-fold, as compared to a GST-π siRNA without deoxynucleotides in the duplex region.

These data show that GST-π siRNAs having a structure with three to four deoxynucleotides located at positions 5-8, or at positions 1, 3, 5 and 7, or at positions 3, 5 and 7 in the seed region of the antisense strand provided unexpectedly increased gene knockdown activity as compared to a GST-π siRNA without deoxynucleotides in the duplex region.

The activities shown in Table 18 for GST-π siRNAs having three to four deoxynucleotides in the seed region of the antisense strand were in the range 30-100 μM, which is exceptionally suitable for many uses, including as a drug agent to be used in vivo.

Example 7: The structure of GST-π siRNAs containing one or more 2′-deoxy-2′-fluoro substituted nucleotides provided unexpectedly increased gene knockdown activity in vitro.

In vitro transfection was performed in an A549 cell line to determine knockdown efficacy for GST-π siRNAs based on structure BU2′ (SEQ ID NOs: 131 and 157). Dose dependent knockdown of GST-π mRNA was observed with the GST-π siRNAs based on structure BU2′, as shown in Table 19.

TABLE 19
Dose dependent knockdown of GST-π mRNA in an A549 cell
line for GST-π siRNAs based on structure BU2′

| GST-π siRNA structure | IC50 (pM) |
|---|---|
| BU2 with no 2′-F deoxynucleotides | 31 |
| (SEQ ID NOs: 61 and 126) | |
| BU2 with seven 2′-F deoxynucleotides, one in | 3 |
| position 1 at the 3′ end of the antisense strand (SEQ | |
| ID NOs: 148 and 174) | |
| BU2 with four 2′-F deoxynucleotides, one in position | 11 |
| 1 at the 3′ end of the antisense strand (SEQ ID | |
| NOs: 147 and 173) | |
| BU2 with one 2′-F deoxynucleotide in position 1 at | 13 |
| the 3′ end of the antisense strand (SEQ ID NOs: 144 | |
| and 170) | |

As shown in Table 19, the activities of GST-π siRNAs based on structure BU2′ having one or more 2′-F deoxynucleotides were surprisingly increased by up to 10-fold, as compared to a GST-π siRNA without 2′-F deoxynucleotides.

These data show that GST-π siRNAs having a structure with one or more 2′-F deoxynucleotides provided unexpectedly increased gene knockdown activity as compared to a GST-π siRNA without a 2′-F deoxynucleotide.

The activities shown in Table 19 for GST-π siRNAs having one or more 2′-F deoxynucleotides were in the range 3 to 13 μM, which is exceptionally suitable for many uses, including as a drug agent to be used in vivo.

Example 8: The structure of GST-π siRNAs containing one or more 2′-deoxy-2′-fluoro substituted nucleotides provided unexpectedly increased gene knockdown activity in vitro.

In vitro transfection was performed in an A549 cell line to determine knockdown efficacy for GST-π siRNAs based on structure B13′ (SEQ ID NOs: 207 and 222). Dose dependent knockdown of GST-π mRNA was observed with the GST-π siRNAs based on structure B13′, as shown in Table 20.

TABLE 20
Dose dependent knockdown of GST-π mRNA in an A549 cell
line for GST-π siRNAs based on structure B13′

| GST-π siRNA structure | IC50 (pM) |
|---|---|
| B13 with no 2′-F deoxynucleotides | 17 |
| (SEQ ID NOs: 50 and 115) | |
| B13 with three 2′-F deoxynucleotides located in non- | 6 |
| overhang positions (SEQ ID NOs: 219 and 234) | |

As shown in Table 20, the activity of a GST-π siRNA based on structure B13′ having three 2′-F deoxynucleotides located in non-overhang positions was surprisingly increased by about 3-fold, as compared to a GST-π siRNA without 2′-F deoxynucleotides.

These data show that GST-π siRNAs having a structure with one or more 2′-F deoxynucleotides provided unexpectedly increased gene knockdown activity as compared to a GST-π siRNA without a 2′-F deoxynucleotide.

The activity shown in Table 20 for GST-π siRNAs having one or more 2′-F deoxynucleotides was in the picomolar range at 6 μM, which is exceptionally suitable for many uses, including as a drug agent to be used in vivo.

Example 9: Orthotopic A549 lung cancer mouse model. The GST-π siRNAs of this invention can exhibit profound reduction of orthotopic lung cancer tumors in vivo. In this example, a GST-π siRNA provided gene knockdown potency in vivo when administered in a liposomal formulation to the orthotopic lung cancer tumors in athymic nude mice.

In general, an orthotopic tumor model can exhibit direct clinical relevance for drug efficacy and potency, as well as improved predictive ability. In the orthotopic tumor model, tumor cells are implanted directly into the same kind of organ from which the cells originated.

The anti-tumor efficacy of the siRNA formulation against human lung cancer A549 was evaluated by comparing the final primary tumor weights measured at necropsy for the treatment group and the vehicle control group.

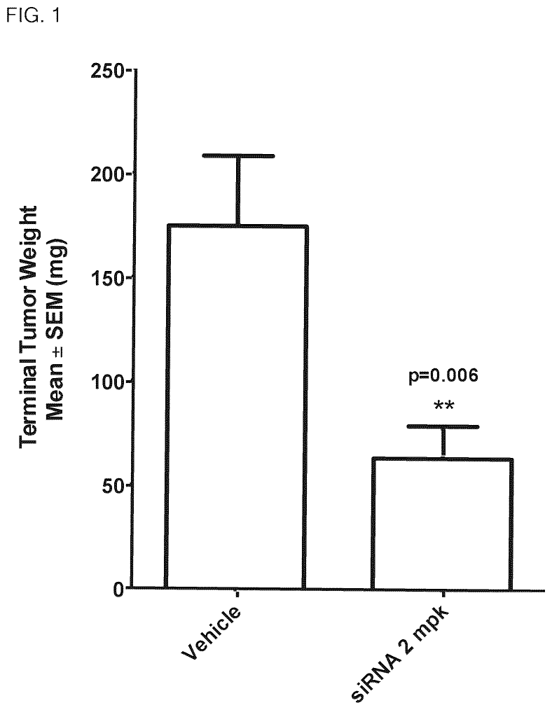

FIG. 1 shows orthotopic lung cancer tumor inhibition in vivo for a GST-π siRNA based on structure BU2 (SEQ ID NOs: 61 and 126). An orthotopic A549 lung cancer mouse model was utilized with a relatively low dose at 2 mg/kg of the siRNA targeted to GST-π.

The GST-π siRNA showed significant and unexpectedly advantageous lung tumor inhibition efficacy in this six-week study. As shown in FIG. 1, after 43 days, the GST-π siRNA showed markedly advantageous tumor inhibition efficacy, with final tumor average weights significantly reduced by 2.8-fold as compared to control.

For this study, male NCr nu/nu mice, 5-6 weeks old, were used. The experimental animals were maintained in a HEPA filtered environment during the experimental period. The siRNA formulations were stored at 4° C. before use, and warmed to room temperature 10 minutes prior to injection in mouse.

For this A549 human lung cancer orthotopic model, on the day of surgical orthotopic implantation (SOI), the stock tumors were harvested from the subcutaneous site of animals bearing A549 tumor xenograft and placed in RPMI-1640 medium. Necrotic tissues were removed and viable tissues were cut into 1.5-2 mm³ pieces. The animals were anesthetized with isoflurane inhalation and the surgical area was sterilized with iodine and alcohol. A transverse incision approximately 1.5 cm long was made in the left chest wall of the mouse using a pair of surgical scissors. An intercostal incision was made between the third and the fourth rib and the left lung was exposed. One A549 tumor fragment was transplanted to the surface of the lung with an 8-0 surgical suture (nylon). The chest wall was closed with a 6-0 surgical suture (silk). The lung was re-inflated by intrathoracic puncture using a 3 cc syringe with a 25 G×1½ needle to draw out the remaining air in the chest cavity. The chest wall was closed with a 6-0 surgical silk suture. All procedures of the operation described above were performed with a 7× magnification microscope under HEPA filtered laminar flow hoods.

Three days after tumor implantation, the model tumor-bearing mice were randomly divided into groups of ten mice per group. For the group of interest, treatment of the ten mice was initiated three days after tumor implantation.

For the group of interest, the formulation was (Ionizable lipid:cholesterol:DOPE:DOPC:DPPE-PEG-2K:DSPE-PEG-2K), a liposomal composition. The liposomes encapsulated the GST-π SiRNA.

For the study endpoint, the experimental mice were sacrificed forty-two days after treatment initiation. Primary tumors were excised and weighed on an electronic balance for subsequent analysis.

For an estimation of compound toxicity, the mean body weight of the mice in the treated and control groups was maintained within the normal range during the entire experimental period. Other symptoms of toxicity were not observed in the mice.

Example 10: The GST-π siRNAs of this invention exhibited profound reduction of cancer xenograft tumors in vivo. The GST-π siRNAs provided gene knockdown potency in vivo when administered in a liposomal formulation to the cancer xenograft tumors.

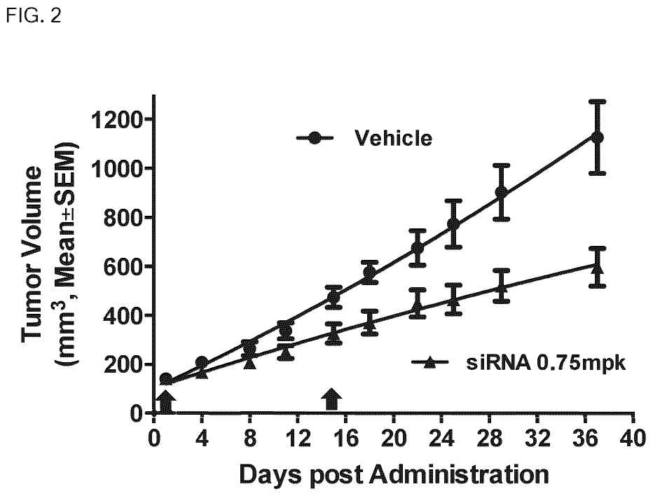

FIG. 2 shows tumor inhibition efficacy for a GST-π siRNA (SEQ ID Nos: 156 and 182). A cancer xenograft model was utilized with a relatively low dose at 0.75 mg/kg of siRNA targeted to GST-π.

The GST-π siRNA showed significant and unexpectedly advantageous tumor inhibition efficacy within a few days after administration. After 36 days, the GST-π siRNA showed markedly advantageous tumor inhibition efficacy, with tumor volume reduced by 2-fold as compared to control.

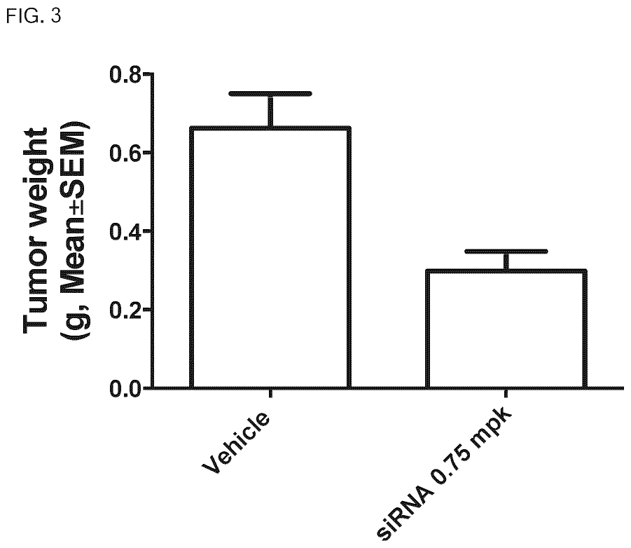

As shown in FIG. 3, the GST-π siRNA demonstrated significant and unexpectedly advantageous tumor inhibition efficacy at the endpoint day. In particular, tumor weight was reduced by more than 2-fold.

The GST-π siRNA was administered in two injections (day 1 and 15) of a liposomal formulation having the composition (Ionizablelipid:Cholesterol:DOPE:DOPC:DPPE-PEG-2K) (25:30:20:20:5).

For the cancer xenograft model, an A549 cell line was obtained from ATCC. The cells were maintained in culture medium supplemented with 10% Fetal Bovine Serum and 100 U/ml penicillin and 100 μg/ml streptomycin. Cells were split 48 hrs before inoculation so that cells were in log phase growth when harvested. Cells were lightly trypsinized with trypsin-EDTA and harvested from tissue culture. The number of viable cells was counted and determined in a hemocytometer in the presence of trypan blue (only viable cells are counted). The cells were resuspended to a concentration of 5×10⁷/ml in media without serum. Then the cell suspension was mixed well with ice thawed BD matrigel at 1:1 ratio for injection.

Mice were Charles River Laboratory Athymic Nude (nu/nu) Female Mice, immuno-compromised, 6-8 weeks old, 7-8 mice per group.

For tumor model preparation, each mouse was inoculated subcutaneously in the right flank with 0.1 ml an inoculum of 2.5×10⁶ of A549 cells using a 25 G needle and syringe, one inoculum per mouse. Mice were not anesthetized for inoculation.

For tumor volume measurements and randomization, tumor size was measured to the nearest 0.1 mm. Tumor volumes were calculated using the formula: Tumor volume=length×width²/2. Once the established tumors reached approximately 120-175 mm³, average tumor volume was about 150 mm³, the mice were assigned into the various vehicle control and treatment groups such that the mean tumor volumes in the treated groups were within 10% of the mean tumor volume in the vehicle control group, ideally, the CV % of tumor volume was less than 25%. On the same day, test articles and control vehicle were administered according to the dosing regimen. Tumor volumes were monitored three times for week 1, twice for the rest of weeks, including the day of study termination.

For dosage administration, on the dosing day, the test articles were taken out from −80° C. freezer and thawed on ice. Before applied to syringes, the bottle containing formulation was reverted by hands for a few times. All test articles were dosed at 0.75 mg/kg by IV, q2w×2, at 10 ml/kg.

For body weight, mice were weighed to the nearest 0.1 g. Body weights were monitored and recorded daily within 7 days post dosing for first dose. Body weights were monitored and recorded twice for weeks, for the rest of weeks, including the day of study termination.

For tumors collection, on 28 days post first dosing, tumor volume was measured, and tumor was dissected for weight measurement, and stored for PD biomarker study. Tumor weight was recorded.

Example 11: The GST-π siRNAs of this invention demonstrated increased cancer cell death by apoptosis of cancer cells in vitro. The GST-π SiRNAs provided GST-π knockdown, which resulted in upregulation of PUMA, a biomarker for apoptosis and associated with loss in cell viability.

GST-π SiRNA SEQ ID NOs: 156 and 182, which contained a combination of deoxynucleotides in the seed region, a 2′-F substituted deoxynucleotide, and 2′-OMe substituted ribonucleotides, provided unexpectedly increased apoptosis of cancer cells.

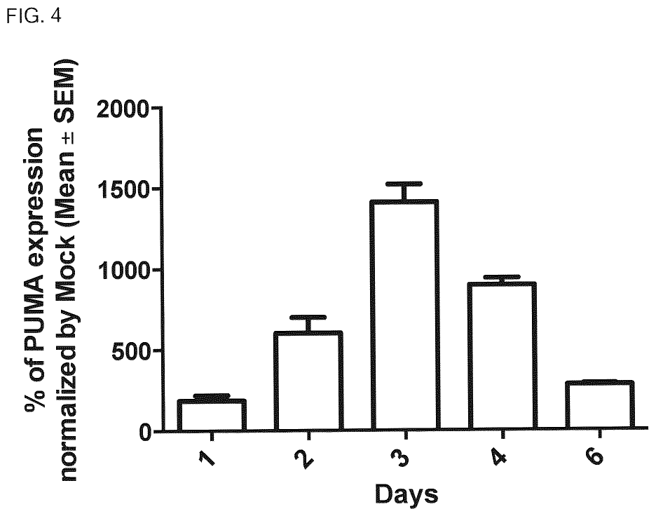

The level of expression of PUMA for GST-π SiRNA SEQ ID NOs: 156 and 182 was measured as shown in FIG. 4. In FIG. 4, the expression of PUMA was greatly increased from 2-4 days after transfection of the GST-π siRNA.

These data show that the structure of GST-π siRNAs containing a combination of deoxynucleotides in the seed region, a 2′-F substituted deoxynucleotide, and 2′-OMe substituted ribonucleotides provided unexpectedly increased apoptosis of cancer cells.

The protocol for the PUMA biomarker was as follows. One day before transfection, cells were plated in a 96-well plate at 2×10³ cells per well with 100 μl of DMEM (HyClone Cat. #SH30243.01) containing 10% FBS and cultured in a 37° C. incubator containing a humidified atmosphere of 5% CO₂ in air. Next day, before transfection the medium was replaced with 90 μl of Opti-MEM I Reduced Serum Medium (Life Technologies Cat. #31985-070) containing 2% FBS. Then, 0.2 μl of Lipofectamine RNAiMAX (Life Technologies Cat. #13778-100) were mixed with 4.8 μl of Opti-MEM I for 5 minutes at room temperature. 1 μl of the GST-siRNA (stock conc. 1 μM) was mixed with 4 μl of Opti-MEM I and combined with the RNAiMAX solution and then mixed gently. The mixture was incubated for 10 minutes at room temperature to allow the RNA-RNAiMAX complexes to form. 10 μl of RNA-RNAiMAX complexes were added per well, to final concentration of the siRNA 10 nM. The cells were incubated for 2 hours and medium changed to fresh Opti-MEM I Reduced Serum Medium containing 2% FBS. For 1, 2, 3, 4, and 6 days post transfection, the cells were washed with ice-cold PBS once and then lysed with 50 μl of Cell-to-Ct Lysis Buffer (Life Technologies Cat. #4391851 C) for 5-30 minutes at room temperature. 5 μl of Stop Solution was added and incubated for 2 minutes at room temperature. PUMA (BBC3, Cat #Hs00248075, Life Technologies) mRNA levels were measured by qPCR with TAQMAN.

Example 12: The GST-π siRNAs of this invention can exhibit profound reduction of cancer xenograft tumors in vivo. The GST-π siRNAs can provide gene knockdown potency in vivo when administered in a liposomal formulation to the cancer xenograft tumors.

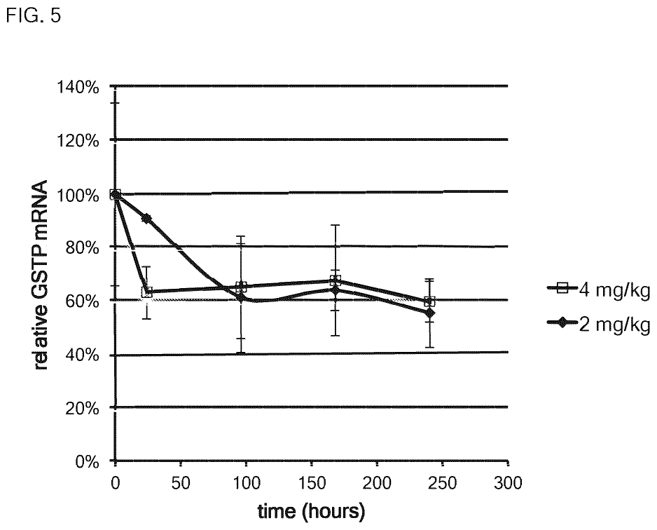

FIG. 5 shows tumor inhibition efficacy for a GST-π siRNA (SEQ ID NOs: 61 and 126). Dose dependent knockdown of GST-π mRNA was observed in vivo with the siRNA targeted to GST-π. A cancer xenograft model was utilized with a relatively low dose at 0.75 mg/kg of siRNA targeted to GST-π.

The GST-π siRNA showed significant and unexpectedly advantageous tumor inhibition efficacy within a few days after administration. As shown in FIG. 5, treatment with a GST-π siRNA resulted in significant reduction of GST-π mRNA expression 4 days after injection in a lipid formulation. At the higher dose of 4 mg/kg, significant reduction of about 40% was detected 24 hours after injection.

The GST-π siRNA was administered in a single injection of 10 mL/kg of a liposomal formulation having the composition (Ionizablelipid:Cholesterol:DOPE:DOPC:DPPE-PEG-2K) (25:30:20:20:5).

For the cancer xenograft model, an A549 cell line was obtained from ATCC. The cells were maintained in RPMI-1640 supplemented with 10% Fetal Bovine Serum and 100 U/ml penicillin and 100 μg/ml streptomycin. Cells were split 48 hrs before inoculation so that cells were in log phase growth when harvested. Cells were lightly trypsinized with trypsin-EDTA and harvested from tissue culture. The number of viable cells was counted and determined in a hemocytometer in the presence of trypan blue (only viable cells are counted). The cells were resuspended to a concentration of 4×10⁷/ml in RPMI media without serum. Then the cell suspension was mixed well with ice thawed BD matrigel at 1:1 ratio for injection.

Mice were Charles River Laboratory Athymic Nude (nu/nu) Female Mice, immuno-compromised, 6-8 weeks old, 3 mice per group.

For tumor model preparation, each mouse was inoculated subcutaneously in the right flank with 0.1 ml an inoculum of 2×10⁶ of A549 cells using a 25 G needle and syringe, one inoculum per mouse. Mice were not anesthetized for inoculation.

For tumor volume measurements and randomization, tumor size was measured to the nearest 0.1 mm. Tumor volumes were calculated using the formula: Tumor volume=length×width²/2. Tumor volumes were monitored twice a week. Once the established tumors reached approximately 350-600 mm³, the mice were assigned into groups with varied time points. On the same day, test articles were administered according to the dosing regimen.

For dosage administration, on the day when the established tumors reached approximately 350-600 mm³, the test articles were taken out from 4° C. fridge. Before being applied to syringes, the bottle containing formulation was reverted by hand for a few times to make a homogeneous solution.

For body weight, mice were weighed to the nearest 0.1 g. Body weights were monitored and recorded twice for weeks, for the rest of weeks, including the day of study termination.

For tumors collection, animals were sacrificed by overdosed CO₂ and tumors were dissected at 0, 24, 48, 72, 96 (optional), and 168 hours following the dosing. Tumors were first wet weighted, and then separated into three parts for KD, distribution and biomarker analysis. The samples were snap frozen in liquid nitrogen and stored at −80° C. until ready to be processed.

Example 13: The GST-π siRNAs of this invention inhibited pancreatic cancer xenograft tumors in vivo. The GST-π siRNAs provided gene knockdown potency in vivo when administered in a liposomal formulation to the pancreatic cancer xenograft tumors.

In this xenograft model, each mouse was inoculated subcutaneously in the right flank with 0.1 ml an inoculum of 2.5×10⁶ of PANC-1 cells. Athymic nude female mice, 6 to 8 weeks, Charles River, were used. Tumor size was measured to the nearest 0.1 mm. Once the established tumors reached approximately 150-250 mm³ (average tumor volume at about 200 mm³), the mice were assigned into the various vehicle control and treatment groups such that the mean tumor volumes in the treated groups were within 10% of the mean tumor volume in the vehicle control group. On the same day, test articles and control vehicle were administered according to the dosing regimen. Tumor volumes were monitored three times for week 1, twice for the rest of weeks, including the day of study termination.

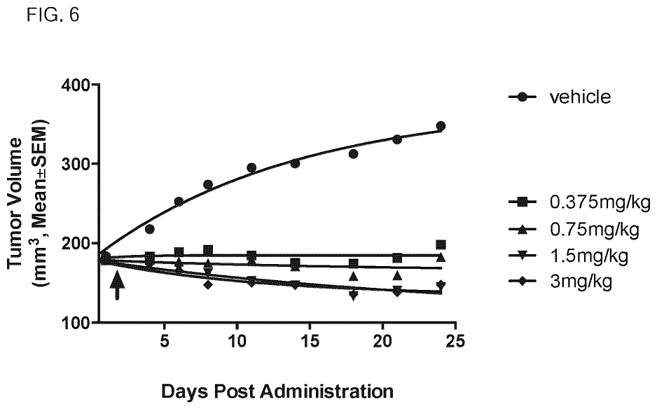

FIG. 6 shows tumor inhibition efficacy for a GST-π SiRNA (SEQ ID Nos: 61 and 126). As shown in FIG. 6, a dose response was obtained with doses ranging from 0.375 mg/kg to 3 mg/kg of siRNA targeted to GST-π. The GST-π siRNA showed significant and unexpectedly advantageous tumor inhibition efficacy within a few days after administration. Thus, the GST-π siRNA demonstrated significant and unexpectedly advantageous tumor inhibition efficacy at the endpoint.

The GST-π siRNAs were administered in a liposomal formulation having the composition (Ionizable lipid:cholesterol:DOPE:DOPC:DPPE-PEG-2K) (25:30:20:20:5).

Example 14: The GST-π siRNAs of this invention exhibited increased serum stability.

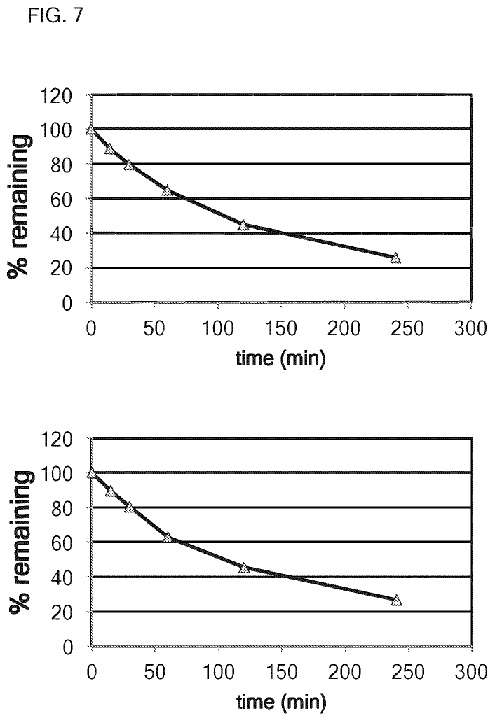

FIG. 7 shows incubation in human serum and detection of remaining siRNA at various time points by HPLS/LCMS. As shown in FIG. 7, the half-life (ty2) in serum for both the sense strand (FIG. 7, top) and antisense strand (FIG. 7, bottom) of a GST-π SiRNA (SEQ ID Nos: 61 and 126) was about 100 minutes.

Example 15: The GST-π siRNAs of this invention exhibited enhanced stability in formulation in plasma.

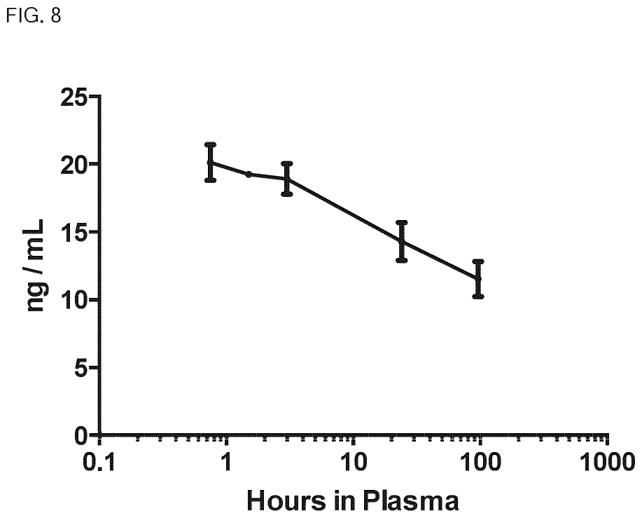

FIG. 8 shows incubation of formulation in plasma and detection of remaining siRNA at various time points. As shown in FIG. 8, the half-life (t_{1/2}) in plasma of a formulation of GST-π SiRNA (SEQ ID Nos: 61 and 126) was significantly longer than 100 hours.

The GST-π siRNA was prepared in a liposomal formulation having the composition (Ionizing lipid:cholesterol:DOPE:DOPC:DPPE-PEG-2K) (25:30:20:20:5). The z-average size for the liposomal nanparticles was 40.0 nm, and the siRNA was 91% encapsulated.

The formulation was incubated in 50% human serum in PBS for 40 min, 1.5h, 3 h, 24 h, and 96h. The amount of the GST-π siRNA was determined by an ELISA-based assay.

Example 16: The GST-π siRNAs of this invention exhibited reduced off target effects by the passenger strand.

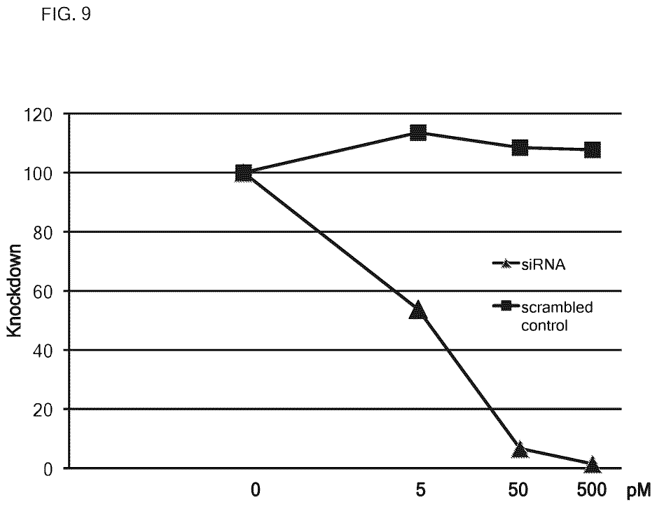

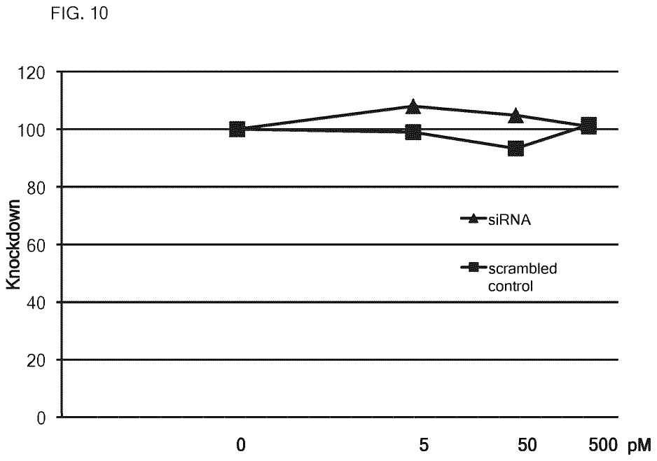

For the GST-π SiRNA (SEQ ID Nos: 156 and 182), FIG. 9 shows that in vitro knockdown for the guide strand was approximately exponential, as compared to a control with scrambled sequence that exhibited no effect. The IC50 of this siRNA was measured at 5 μM. FIG. 10 shows in vitro knockdown for the passenger strand of the same GST-π siRNA. As shown in FIG. 10, the passenger strand off target knockdown for the GST-π siRNA was greatly reduced, by more than 100-fold.

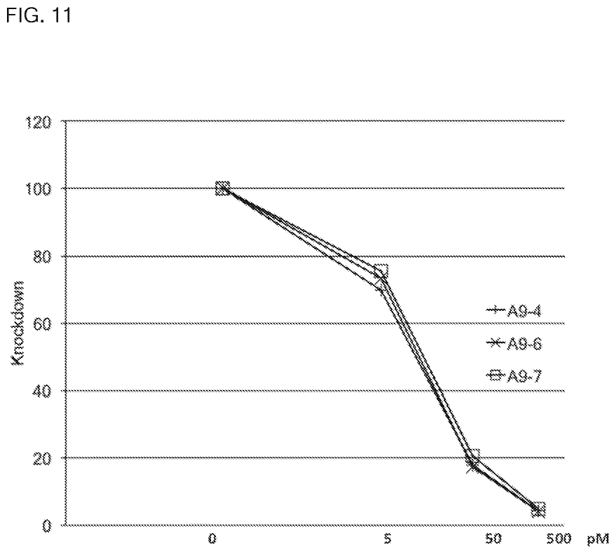

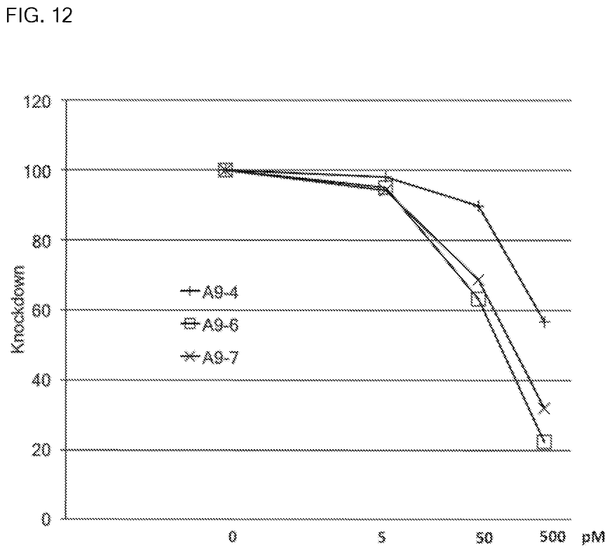

For the GST-π siRNAs (SEQ ID Nos: 187 and 199), (SEQ ID Nos: 189 and 201), and (SEQ ID Nos: 190 and 202), FIG. 11 shows that the in vitro knockdowns for the guide strands were approximately exponential. The IC50s of these siRNAs were measured at 6, 7, and 5 μM, respectively. As shown in FIG. 12, the in vitro knockdowns for the passenger strands of these GST-π siRNAs were significantly reduced by at least 10-fold. All of these GST-π siRNAs had deoxynucleotides in the seed region of the duplex region, with no other modifications in the duplex region.

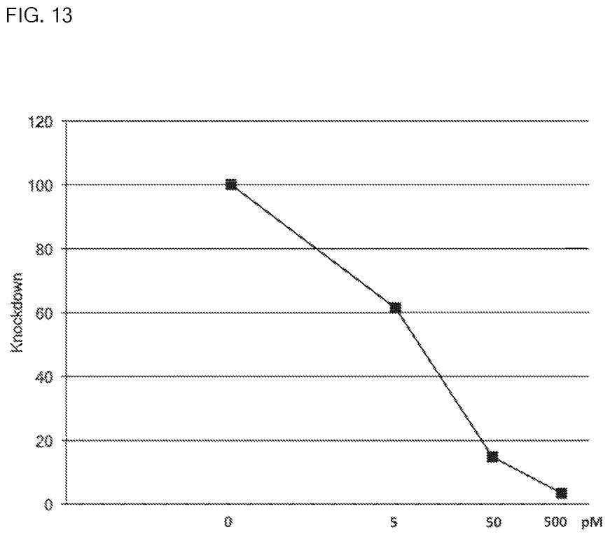

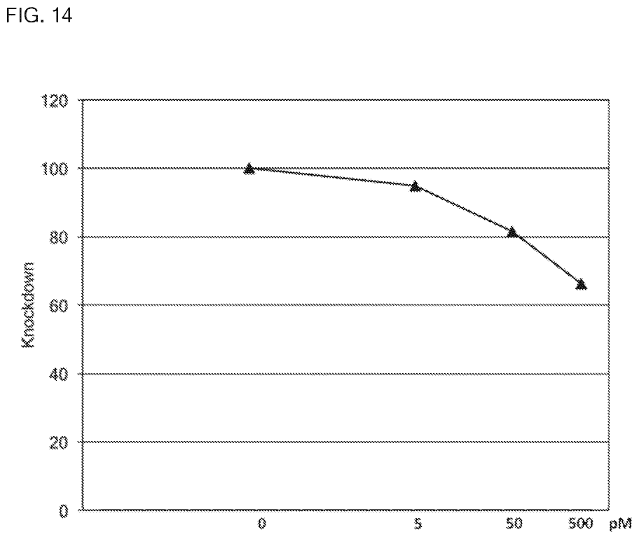

For the GST-π siRNAs (SEQ ID Nos: 217 and 232), FIG. 13 shows that the in vitro knockdown for the guide strand of this highly active GST-π siRNA was approximately exponential. The IC50 of this siRNA was measured at 11 μM. As shown in FIG. 14, the in vitro knockdown for the passenger strand of this GST-π siRNA was significantly reduced by more than 100-fold. This GST-siRNA had deoxynucleotides in the seed region of the duplex region, with no other modifications in the duplex region.

Off-target effects were determined using the expression reporter plasmid psiCHECK-2, which encodes the Renilla luciferase gene. (Dual-Luciferase Reporter Assay System, Promega, Cat #:E1960). The siRNA concentration was typically 50 μM. Protocol: Day 1, HeLa cell seeded at 5 to 7.5×10³/100 μl/well. Day 2, co-transfection with cell confluence about 80%. Day 3, cells harvested for luciferase activity measurement. Luciferase activity was measured using Promega's Luciferase Assay System (E4550), according to manufacturer's protocol.

The psiCHECK-2 vector enabled monitoring of changes in expression of a target gene fused to the reporter gene of Renilla luciferase. The siRNA constructs were cloned into the multiple cloning region, and the vector was cotransfected with the siRNA into HeLa cells. If a specific siRNA binds to the target mRNA and initiates the RNAi process, the fused Renilla luciferase: construct mRNA will be cleaved and subsequently degraded, decreasing the Renilla luciferase signal.

For example, the plasmid inserts for siRNAs with the BU2′ structure were as follows:

PsiCHECK-2 (F) plasmid insert:

SEQ ID NO.: 451

ctcgag gggcaacTGAAGCCTTTTGAGACCCTGcTgTcccag
gcggccgc
PsiCHECK-2 (R) plasmid insert:

SEQ ID NO.: 452

ctcgag cTgggacagCAGGGTCTCAAAAGGCTTCagTTgccc
gcggccgc

Example 17: The GST-π siRNAs of this invention exhibited advantageously reduced miRNA-like off target effects, which are seed-dependent unintended off-target gene silencing.

For the GST-π siRNAs (SEQ ID Nos: 156 and 182), (SEQ ID Nos: 187 and 199), (SEQ ID Nos: 189 and 201), (SEQ ID Nos: 190 and 202), and (SEQ ID Nos: 217 and 232), off target activity mimicking miRNA was found to be essentially negligible. The seed-dependent unintended off-target gene silencing for these GST-π siRNAs was at least 10-fold to 100-fold less than the on-target activity of the guide strand.

For testing miRNA-related off target effects, one to four repeats of seed-matched target sequences complementary to the entire seed-containing region, positions 1-8 of the 5′ end of the antisense strand, but not to the remaining non-seed region, positions 9-21, were introduced into the region corresponding to the 3′UTR of the luciferase mRNA, to determine the efficiency of the seed-dependent unintended off-target effects. Plasmid inserts were used to mimic a miRNA with complete matching in the seed region and mismatches (bulges) in the non-seed region.

For example, the plasmid inserts for siRNAs with the BU2′ structure were as follows:

PsiCHECK-2 (Fmi1) plasmid insert:
SEQ ID NO.: 453
ctcgag gggcaacTCTACGCAAAACAGACCCTGcTgTcccag gcggcc
gc
PsiCHECK-2 (Fmi2) plasmid insert:
SEQ ID NO.: 454
ctcgag gggcaacTCTACGCAAAACAGACCCTGcT CTACGCAAAACAG
ACCCTGcTgTcccag gcggccgc
PsiCHECK-2 (Fmi3) plasmid insert:
SEQ ID NO.: 455
ctcgag gggcaacTCTACGCAAAACAGACCCTGcT CTACGCAAAACAG
ACCCTGcTCTACGCAAAACAGACCCTGcT gTcccag gcggccgc
PsiCHECK-2 (Fmi4) plasmid insert:
SEQ ID NO.: 456
ctcgag gggcaacTCTACGCAAAACAGACCCTGcT CTACGCAAAACAG
ACCCTGcTCTACGCAAAACAGACCCTGcT CTACGCAAAACAGACCCTGc
T gTcccag gcggccgc

The embodiments described herein are not limiting and one skilled in the art can readily appreciate that specific combinations of the modifications described herein can be tested without undue experimentation toward identifying nucleic acid molecules with improved RNAi activity.

Example 18: siRNAs of this invention targeted to p21 were found to be active for gene silencing in vitro. The dose-dependent activities of p21 siRNAs for gene knockdown were found to exhibit an IC50 below about 3 picomolar (pM), and as low as 1 μM.

In vitro transfection was performed in an A549 cell line to determine siRNA knockdown efficacy. Dose dependent knockdown for p21 mRNA was observed with siRNAs of Table 7, as shown in Table 21.

TABLE 21
Dose dependent knockdown for p21 mRNA in an A549 cell line

| P21 siRNA structure | IC50 (pM) |
|---|---|
| 1735 (SEQ ID NOs: 296 and 324) | 0.3 |
| 2042 (SEQ ID NOs: 312 and 340) | 10 |

As shown in Table 21, the activities of p21 siRNAs of Table 7 were in the range 0.3-10 μM, which is suitable for many uses, including as a drug agent to be used in vivo.

Example 19: The structure of p21 siRNAs of this invention having deoxynucleotides located in the seed region of the antisense strand of the siRNA provided unexpectedly and advantageously increased gene knockdown activity.

In vitro transfection was performed in an A549 cell line to determine knockdown efficacy for p21 siRNAs based on structure 1735′ (SEQ ID NOs: 341 and 355). Dose dependent knockdown of p21 mRNA was observed with p21 siRNAs based on structure 1735′ as shown in Table 22.

TABLE 22
Dose dependent knockdown of p21 mRNA in an A549
cell line for p21 siRNAs based on structure 1735′

| P21 siRNA structure | IC50 (pM) |
|---|---|
| 1735 with no deoxynucleotides in the duplex region | 0.3 |
| (SEQ ID NOs: 296 and 324) | |
| 1735 with deoxynucleotides in positions 4, 6, and 8 | 0.05 |
| of the seed region antisense strand, and additional | |
| 2′-OMe nucleotides (SEQ ID NOs: 342 and 356) | |
| 1735 with deoxynucleotides in positions 4, 6, and 8 | 0.001 |
| of the seed region antisense strand, and additional | |
| 2′-OMe nucleotides (SEQ ID NOs: 343 and 357) | |
| 1735 with deoxynucleotides in positions 4, 6, and 8 | 0.1 |
| of the seed region antisense strand, and additional | |
| 2′-OMe nucleotides (SEQ ID NOs: 344 and 358) | |

As shown in Table 22, the activities of p21 siRNAs based on structure 1735′ having three deoxynucleotides in the seed region of the antisense strand were surprisingly and unexpectedly increased by up to 300-fold, as compared to a p21 siRNA without deoxynucleotides in the duplex region.

These data show that p21 siRNAs having a structure with deoxynucleotides in the seed region of the antisense strand provided surprisingly increased gene knockdown activity as compared to a p21 siRNA without deoxynucleotides in the duplex region.

The activities shown in Table 22 for p21 siRNAs having three deoxynucleotides in the seed region of the antisense strand were in the range 0.001 to 0.1 pM, which is exceptionally suitable for many uses, including as a drug agent to be used in vivo.

Example 20: The p21 siRNAs of this invention can exhibit profound reduction of cancer xenograft tumors in vivo. The p21 siRNAs can provide gene knockdown potency in vivo when administered in a liposomal formulation to the cancer xenograft tumors.

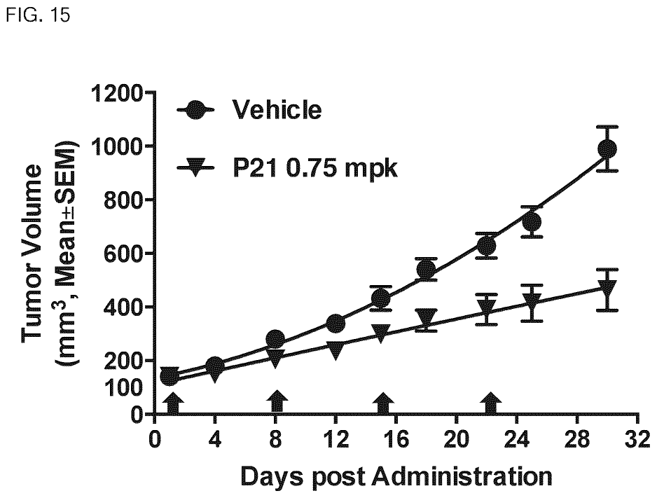

FIG. 15 shows tumor inhibition efficacy for a p21 siRNA (SEQ ID Nos: 341 and 355, where N=U). A cancer xenograft model was utilized with a relatively low dose at 0.75 mg/kg of siRNA targeted to p21.

The p21 siRNA showed significant and unexpectedly advantageous tumor inhibition efficacy within a few days after administration. After 30 days, the p21 siRNA showed markedly advantageous tumor inhibition efficacy, with tumor volume reduced by more than 2-fold as compared to control.

The p21 siRNA was administered at a dosage of 0.75 mg/kg in four injections of 10 mL/kg (day 1, 8, 15 and 22) of a liposomal formulation having the composition (Ionizablelipid:Cholesterol:DOPE:DOPC:DPPE-PEG-2K) (25:30:20:20:5).

For the cancer xenograft model, an A549 cell line was obtained from ATCC. The cells were maintained in culture medium supplemented with 10% Fetal Bovine Serum and 100 U/ml penicillin and 100 μg/ml streptomycin. Cells were split 48 hrs before inoculation so that cells were in log phase growth when harvested. Cells were lightly trypsinized with trypsin-EDTA and harvested from tissue culture. The number of viable cells was counted and determined in a hemocytometer in the presence of trypan blue (only viable cells are counted). The cells were resuspended to a concentration of 5×10⁷/ml in media without serum. Then the cell suspension was mixed well with ice thawed BD matrigel at 1:1 ratio for injection.

Mice were Charles River Laboratory Athymic Nude (nu/nu) Female Mice, immuno-compromised, 6-8 weeks old, 7-8 mice per group.

For tumor model preparation, each mouse was inoculated subcutaneously in the right flank with 0.1 ml an inoculum of 2.5×10⁶ of A549 cells using a 25 G needle and syringe, one inoculum per mouse. Mice were not anesthetized for inoculation.

For tumor volume measurements and randomization, tumor size was measured to the nearest 0.1 mm. Tumor volumes were calculated using the formula: Tumor volume=length×width²/2. Once the established tumors reached approximately 120-175 mm³, average tumor volume was about 150 mm³, the mice were assigned into the various vehicle control and treatment groups such that the mean tumor volumes in the treated groups were within 10% of the mean tumor volume in the vehicle control group, ideally, the CV % of tumor volume was less than 25%. On the same day, test articles and control vehicle were administered according to the dosing regimen. Tumor volumes were monitored three times for week 1, twice for the rest of weeks, including the day of study termination.

For dosage administration, on the dosing day, the test articles were taken out from −80° C. freezer and thawed on ice. Before applied to syringes, the bottle containing formulation was reverted by hands for a few times. All test articles were dosed at 0.75 mg/kg by IV.

For body weight, mice were weighed to the nearest 0.1 g. Body weights were monitored and recorded daily within 7 days post dosing for first dose. Body weights were monitored and recorded twice for weeks, for the rest of weeks, including the day of study termination.

For tumors collection, on 28 days post first dosing, tumor volume was measured, and tumor was dissected for weight measurement, and stored for PD biomarker study. Tumor weight was recorded.

The embodiments described herein are not limiting and one skilled in the art can readily appreciate that specific combinations of the modifications described herein can be tested without undue experimentation toward identifying nucleic acid molecules with improved RNAi activity.

Example 21: siRNAs of this invention targeted to MCL1 are prepared and are found to be active for gene silencing in vitro. The dose-dependent activities of MCL1 siRNAs for gene knockdown are found to exhibit an IC50 below about 100 picomolar (pM).

In vitro transfection is performed in an A549 cell line to determine siRNA knockdown efficacy. Dose dependent knockdown for MCL1 mRNA is observed with siRNAs of Table 11.

Example 22: siRNAs of this invention targeted to ARAF are prepared and are found to be active for gene silencing in vitro. The dose-dependent activities of ARAF siRNAs for gene knockdown are found to exhibit an IC50 below about 100 picomolar (pM).

In vitro transfection is performed in an A549 cell line to determine siRNA knockdown efficacy. Dose dependent knockdown for ARAF mRNA is observed with siRNAs of Table 12.

All publications, patents and literature specifically mentioned herein are incorporated by reference in their entirety for all purposes.

It is understood that this invention is not limited to the particular methodology, protocols, materials, and reagents described, as these may vary. It is also to be understood that the terminology used herein is for the purpose of describing particular embodiments only, and is not intended to limit the scope of the present invention. It will be readily apparent to one skilled in the art that varying substitutions and modifications can be made to the description disclosed herein without departing from the scope and spirit of the description, and that those embodiments are within the scope of this description and the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprises,” “comprising”, “containing,” “including”, and “having” can be used interchangeably, and shall be read expansively and without limitation.

Recitation of ranges of values herein are merely intended to serve as a shorthand method of referring individually to each separate value falling within the range, unless otherwise indicated herein, and each separate value is incorporated into the specification as if it were individually recited herein. For Markush groups, those skilled in the art will recognize that this description includes the individual members, as well as subgroups of the members of the Markush group.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever.

All of the features disclosed in this specification may be combined in any combination. Each feature disclosed in this specification may be replaced by an alternative feature serving the same, equivalent, or similar purpose.

What is claimed is:

1. A nucleic acid molecule, wherein:
a) the molecule has a polynucleotide sense strand and a polynucleotide antisense strand;
b) each strand of the molecule is from 15 to 30 nucleotides in length;
c) a contiguous region of from 15 to 30 nucleotides of the antisense strand is complementary to a sequence of a mRNA;
d) at least a portion of the sense strand is complementary to at least a portion of the antisense strand, and the molecule has a duplex region of from 15 to 30 nucleotides in length, wherein one or more of the nucleotides in the duplex region at positions 3 to 8 from the 5′ end of the antisense strand are deoxynucleotides, and all the remaining nucleotides in the duplex region of the antisense strand are not deoxynucleotides; and
wherein the molecule is modified or chemically-modified at least at the 5′ end or the 3′ end of the antisense strand.

2. The molecule of claim 1, wherein three to six nucleotides in the duplex region at positions 3 to 8 from the 5′ end of the antisense strand are deoxynucleotides.

3. The molecule of claim 1, wherein the antisense strand has deoxynucleotides in a plurality of positions, the plurality of positions being one of the following:
each of positions 4, 6 and 8, from the 5′ end of the antisense strand;
each of positions 3, 5 and 7, from the 5′ end of the antisense strand;
each of positions 1, 3, 5 and 7, from the 5′ end of the antisense strand;
each of positions 3-8, from the 5′ end of the antisense strand; and
each of positions 5-8, from the 5′ end of the antisense strand.

4. The molecule of claim 1, wherein the molecule is an RNAi molecule active for modulating expression of the mRNA.

5. The molecule of claim 1, wherein the mRNA is a human mRNA.

6. The molecule of claim 1, wherein each strand of the molecule is from 18 to 22 nucleotides in length.

7. The molecule of claim 1, wherein the duplex region is 19 nucleotides in length.

8. The molecule of claim 1, wherein the polynucleotide sense strand and the polynucleotide antisense strand are connected as a single strand, and form a duplex region connected at one end by a loop.

9. The molecule of claim 1, wherein the molecule has a blunt end.

10. The molecule of claim 1, wherein the molecule has one or more 3′ overhangs.

11. The molecule of claim 1, wherein one or more of the nucleotides in the duplex region is modified or chemically-modified.

12. The molecule of claim 11, wherein the modified or chemically-modified nucleotides are 2′-O-alkyl substituted nucleotides, 2′-deoxy-2′-fluoro substituted nucleotides, phosphorothioate nucleotides, locked nucleotides, or any combination thereof.

13. A pharmaceutical composition comprising the molecule of claim 1 and a pharmaceutically acceptable carrier.

14. The pharmaceutical composition of claim 13, wherein the carrier comprises a lipid molecule or liposome.

15. A vector or an isolated cell comprising the molecule of claim 1.

16. A method for treating or ameliorating a disease in a subject in need by gene silencing, the method comprising administering to the subject the pharmaceutical composition of claim 13.

17. A composition of claim 13 for use in medical therapy.

18. A composition of claim 13 for use in the treatment of the human or animal body.

19. The molecule of claim 1, wherein the modified or chemically-modified molecule comprises a 5′-propylamine end, a 5′-phosphorylated end, a 3′-puromycin end, or a 3′-biotin end.

20. The molecule of claim 1, wherein the modified or chemically-modified molecule comprises a 2′-fluoro substituted ribonucleotide, a 2′-OMe substituted ribonucleotide, a 2′-deoxy ribonucleotide, a 2′-amino substituted ribonucleotide, or a 2′-thio substituted ribonucleotide.
